(19) United States
(12) Patent Application Publication
(10) Pub. No.: US 2026/0224676 A1
(43) Pub. Date: Aug. 6, 2026

(54) TAU AND AMYLOID BETA PEPTIDE IMMUNOGEN COMPOSITIONS AND RELATED METHODS

(71) Applicant: Vaxxinity, Inc., Merritt Island, FL (US)

(72) Inventors: Justin Boyd, Merritt Island, FL (US); Jean-Cosme Dodart, Merritt Island, FL (US); Cyrille Vivian Gineste, Merritt Island, FL (US)

(21) Appl. No.: 19/151,140

(22) PCT Filed: Jan. 17, 2024
(86) PCT No.: PCT/US2024/011843

§ 371 Date: Jul. 25, 2025

Related U.S. Application Data
(60) Provisional application No. 63/481,744, filed on Jan. 26, 2023.

(51) Int. Cl. A61K 39/00; A61K 38/00; A61K 39/385; A61P 25/28; C07K 14/47

(52) U.S. Cl. CPC A61K 39/0007; A61K 39/385; A61P 25/28; C07K 14/4711; A61K 38/00; A61K 2039/55561; A61K 2039/575; A61K 2039/6037; A61K 2039/6068; A61K 2039/6075; C07K 2319/40

(57) ABSTRACT

The present disclosure is directed to improved peptide immunogen constructs targeting portions of the Tau protein for the treatment and/or prevention of tauopathies. The present disclosure is also directed to improved peptide immunogen constructs targeting portions of the amyloid beta protein for the treatment and/or prevention of tauopathies. The present disclosure is also directed to compositions and kits containing the peptide immunogen constructs, in particular compositions and kits comprising combinations of various peptide immunogen constructs. Methods of making and using the peptide immunogen constructs, and antibodies produced by the peptide immunogen constructs, are also provided herein. In some aspects, the disclosure is directed to compositions comprising peptide immunogen constructs based on portions of the Tau protein and peptide immunogen constructs based on portions of the amyloid beta protein, and use of these peptide immunogen constructs and compositions in therapeutic methods.

### CROSS-REFERENCE TO RELATED APPLICATIONS

This application claims the benefit of U.S. Provisional Patent Application No. 63/481,744, filed on Jan. 26, 2023, and U.S. Patent Application No. 63/453,677, filed on Mar. 21, 2023, each of which is incorporated by reference herein in its entirety.

### REFERENCE TO SEQUENCE LISTING

This application is being filed electronically and includes an electronically submitted sequence listing in ST.26 (XML) format, which is entitled “VXH-11025_SL.XML,” created on Jan. 16, 2024, and has a size of 216,916 bytes. The sequence listing in ST.26 (XML) format is part of the specification and is incorporated by reference herein in its entirety.

### FIELD OF THE INVENTION

This disclosure relates to peptide immunogen constructs and compositions based on portions of the Tau protein and formulations thereof for the prevention and treatment of tauopathies. This disclosure also relates to peptide immunogen constructs and compositions based on portions of the amyloid beta protein and formulations thereof for the prevention and treatment of tauopathies. The disclosure further relates to compositions comprising peptide immunogen constructs based on portions of the Tau protein and peptide immunogen constructs based on portions of the amyloid beta protein, and use of these compositions in therapeutic methods.

### BACKGROUND OF THE INVENTION

Tau proteins are abundant in neurons of the central nervous system and play a role in stabilization of microtubules. Pathologies and dementias of the nervous system, such as Alzheimer's disease (AD), are associated with Tau proteins that have become defective and no longer stabilize microtubules properly. Additional neurodegenerative diseases, referred to as tauopathies, are characterized by abundant filamentous Tau. These diseases include tangle-only dementia, chronic traumatic encephalopathy (CTE), argyrophilic grain disease, progressive supranuclear palsy, corticobasal degeneration, globular glial tauopathy, and Pick's disease.

Amyloid beta (Aβ) peptides are the main components of amyloid plaques found in the brains of people with Alzheimer's disease, and oligomers of these peptides are toxic to nerve cells. There is a need for the development of Tau- and amyloid beta-directed therapies for use in the treatment of patients suffering from diseases such as Alzheimer's disease and tauopathies.

### SUMMARY OF THE INVENTION

The present disclosure is directed to individual peptide immunogen constructs targeting portions of the Tau protein for the treatment and/or prevention of tauopathies. The present disclosure is also directed to individual peptide immunogen constructs targeting portions of the amyloid beta protein for the treatment and/or prevention of tauopathies. The present disclosure is also directed to compositions containing one, two or more of the peptide immunogen constructs, methods of making and using the peptide immunogen constructs, and antibodies produced by the peptide immunogen constructs. The disclosure is further directed to compositions comprising combinations of one, two or more Tau peptide immunogen constructs, compositions comprising combinations of one, two or more Tau peptide immunogen constructs and one or more amyloid beta (Aβ) peptide immunogen constructs, and methods of using these compositions and combinations in the prevention and treatment of tauopathies, e.g., Alzheimer's disease and other tauopathies described herein.

The disclosed peptide immunogen constructs contain about 15 or more amino acids. The Tau peptide immunogen constructs contain a B cell epitope from portions of the longest isoform of the human Tau protein (GenBank: AGF19246.1) having the amino acid sequence of SEQ ID NO: 60 shown in Table 6. The amyloid beta (Aβ) peptide immunogen constructs contain a B cell epitope from Aβ₁₋₄₂ (SEQ ID NO: 68), e.g., Aβ₁₋₁₄ (SEQ ID NO: 69) (see Table 3 and SEQ ID NOs: 70 and 71, also see the Aβ peptides described in WO 2014/143087, the contents of which are incorporated herein by reference). In some embodiments, the peptide immunogen constructs and/or B cell epitopes contain chemical modifications and/or unnatural amino acids as described herein. The B cell epitopes can be linked to a heterologous T helper cell (Th) epitope derived from a pathogen protein, e.g., through an optional heterologous spacer. The disclosed peptide immunogen constructs stimulate the generation of highly specific antibodies directed against the B cell epitopes. The disclosed peptide immunogen constructs can be used as an immunotherapy for patients suffering from tauopathies (e.g., Alzheimer's disease; also see other tauopathies listed herein).

The B cell epitope portion of the Tau peptide immunogen constructs have amino acid sequences from the full-length Tau protein (SEQ ID NO: 60). In some embodiments, the Tau peptide immunogen construct has any B cell epitope sequence from the Tau protein as described herein. In some embodiments, the Tau peptide immunogen construct has a B cell epitope sequence from the Tau protein which has not been modified. In some embodiments, the Tau peptide immunogen construct has a B cell epitope sequence from the Tau protein which has been modified, e.g., as described herein (for example, by substitution or chemical modification of one or more amino acids). Contemplated modifications include, without limitation, substitution of one or more amino acids with Cysteine to create a disulfide bond, acetylation or N-methylation of one or more amino acids, as described herein. In some embodiments, the Tau B cell epitope has any of the sequences shown in Table 1. In some embodiments, the Tau B cell epitope has any of the sequences shown in Tables 7-10 (in particular, immunogenic Tau B cell epitopes provided in any one of Tables 7-10). In some embodiments, the B cell epitope has a sequence containing any one of SEQ ID NOs: 1-8, 17-30, or 63-67 as shown in Table 1 or any one of the sequences shown in Tables 7-10, or combinations thereof, e.g., SEQ ID NO: 2 and 7. In some embodiments, the B cell epitope has a sequence containing any one of SEQ ID NOs: 72-76 as shown in Table 1.

The B cell epitope portion of the Aβ peptide immunogen constructs can have amino acid sequences from full-length Aβ₁₋₄₂ (SEQ ID NO: 68). In some embodiments, the Aβ peptide immunogen construct has any B cell epitope sequence from the Aβ protein as described herein. In some embodiments, the Aβ peptide immunogen construct has a B cell epitope sequence from the Aβ protein which has not been modified. In some embodiments, the Aβ peptide immunogen construct has a B cell epitope sequence from the Aβ protein which has been modified, e.g., as described herein (for example, by substitution or chemical modification of one or more amino acids). Contemplated modifications include, without limitation, substitution of one or more amino acids with Cysteine to create a disulfide bond, acetylation or N-methylation of one or more amino acids, as described herein. In some embodiments, the Aβ B cell epitope has any of the sequences shown in Table 3. In some embodiments, the Aβ B cell epitope has any of the sequences disclosed in WO2014/143087 and/or U.S. Pat. No. 9,102,752 B2, each of which is incorporated by reference herein in its entirety and specifically for the disclosure of Aβ B cell epitopes (in particular, immunogenic Aβ B cell epitopes provided in WO2014/143087 and/or U.S. Pat. No. 9,102,752 B2). In some embodiments, the B cell epitope has a sequence comprising Aβ₁₋₁₄ (SEQ ID NO: 69) or any other Aβ peptide sequence described in WO 2014/143087. In some embodiments, the amyloid beta B cell epitope has SEQ ID NO:69 or SEQ ID NO:82.

The peptide immunogen constructs of the present disclosure can contain a heterologous Th epitope amino acid sequence derived from a pathogenic protein (e.g., any one of ID NOs: 31-59) as shown in Table 5. In certain embodiments, the heterologous Th epitope is derived from natural pathogens, such as Diphtheria Toxin (SEQ ID NO: 35), Plasmodium Falciparum (SEQ ID NO: 36), Cholera Toxin (SEQ ID NO: 38). In other embodiments, the heterologous Th epitope is an idealized artificial Th epitope derived from Measles Virus Fusion protein (MVF 1 to 5) or Hepatitis B Surface Antigen (HBsAg 1 to 3) in the form of either single sequence or combinatorial sequences (e.g., any one of SEQ ID NOs: 32, 39, or 41-45). In some embodiments, the Th epitope sequence is SEQ ID NO: 44. In some embodiments, the Th epitope sequence is SEQ ID NO: 42. In some embodiments, the Th epitope sequence is SEQ ID NO: 45. In some embodiments, the Th epitope sequence is SEQ ID NO: 97. In any one of the peptide immunogen constructs described herein, the provided Th epitope can be substituted with the Th epitope of any of SEQ ID NOs: 42, 44, 45 or 97, e.g., SEQ ID NO: 44.

In some embodiments, the peptide immunogen constructs contain a B cell epitope from Tau or Aβ covalently linked to a heterologous T helper cell (Th) epitope. In some embodiments, the peptide immunogen constructs contain a B cell epitope from Tau or Aβ linked to a heterologous T helper cell (Th) epitope through an optional heterologous spacer. In some embodiments, the peptide immunogen constructs contain a B cell epitope from Tau or Aβ directly covalently linked to a heterologous T helper cell (Th) epitope.

In certain embodiments, the Tau peptide immunogen constructs contain a B cell antigenic site having the amino acid sequence from Tau (e.g., any one of SEQ ID NOs: 1-8, 17-30, or 63-67, or 202) linked to a heterologous Th epitope derived from a pathogenic protein (e.g., any one of SEQ ID NOs: 31-59 and 97) through an optional heterologous spacer. In some embodiments, the Tau peptide immunogen construct contains a B cell antigenic site having the amino acid sequence of SEQ ID NO: 2. In some embodiments, the Tau peptide immunogen construct contains a B cell antigenic site having the amino acid sequence of SEQ ID NO: 7. In some embodiments, the optional heterologous spacer is a molecule or chemical structure capable of linking two amino acids and/or peptides together. In certain embodiments, the spacer is a naturally occurring amino acid, a non-naturally occurring amino acid, or a combination thereof. In specific embodiments, the Tau peptide immunogen constructs have the amino acid sequence of any one of SEQ ID NOs: 9-16 (e.g., SEQ ID NO: 10 or 15) shown in Table 2 (or a construct in which the positions of the Tau peptide and the Th peptide are reversed).

In certain embodiments, the Tau peptide immunogen constructs contain a B cell antigenic site having the amino acid sequence from Tau (e.g., any one of SEQ ID NOs: 72-76 or 96) linked to a heterologous Th epitope derived from a pathogenic protein (e.g., any one of SEQ ID NOs: 31-59 and 97) through an optional heterologous spacer. In some embodiments, the Tau peptide immunogen construct contains a B cell antigenic site having the amino acid sequence of SEQ ID NO: 72. In some embodiments, the Tau peptide immunogen construct contains a B cell antigenic site having the amino acid sequence of SEQ ID NO: 73. In some embodiments, the Tau peptide immunogen construct contains a B cell antigenic site having the amino acid sequence of SEQ ID NO: 74. In some embodiments, the Tau peptide immunogen construct contains a B cell antigenic site having the amino acid sequence of SEQ ID NO: 75. In some embodiments, the Tau peptide immunogen construct contains a B cell antigenic site having the amino acid sequence of SEQ ID NO: 76. In some embodiments, the optional heterologous spacer is a molecule or chemical structure capable of linking two amino acids and/or peptides together. In certain embodiments, the spacer is a naturally occurring amino acid, a non-naturally occurring amino acid, or a combination thereof. In specific embodiments, the Tau peptide immunogen constructs have the amino acid sequence of any one of SEQ ID NOs: 77-81 shown in Table 2 (or a construct in which the positions of the Tau peptide and the Th peptide are reversed).

In certain embodiments, the Aβ peptide immunogen constructs contain a B cell antigenic site having an amino acid sequence from Aβ₁₋₄₂ (SEQ ID NO: 68) linked to a heterologous Th epitope derived from a pathogenic protein (e.g., any one of SEQ ID NOs: 31-59 and 97) through an optional heterologous spacer. In some embodiments, the optional heterologous spacer is a molecule or chemical structure capable of linking two amino acids and/or peptides together. In certain embodiments, the spacer is a naturally occurring amino acid, a non-naturally occurring amino acid, or a combination thereof. In specific embodiments, the Aβ peptide immunogen construct has the amino acid sequence of one of SEQ ID NOs: 70 or 71 shown in Table 4 (or a construct in which the positions of the Aβ peptide and the Th peptide are reversed).

In certain embodiments, the Aβ peptide immunogen constructs contain a B cell antigenic site having an amino acid sequence of one of SEQ ID Nos: 68, 69, 82-88 as shown in Table 3, linked to a heterologous Th epitope derived from a pathogenic protein (e.g., any one of SEQ ID NOs: 31-59 and 97) through an optional heterologous spacer. In some embodiments, the Aβ peptide immunogen construct contains a B cell antigenic site having the amino acid sequence of SEQ ID NO: 69. In some embodiments, the Aβ peptide immunogen construct contains a B cell antigenic site having the amino acid sequence of SEQ ID NO: 82. In some embodiments, the optional heterologous spacer is a molecule or chemical structure capable of linking two amino acids and/or peptides together. In certain embodiments, the spacer is a naturally occurring amino acid, a non-naturally occurring amino acid, or a combination thereof. In specific embodiments, the Aβ peptide immunogen construct has the amino acid sequence of SEQ ID NOs: 70 or 89 shown in Table 4 (or a construct in which the positions of the Aβ peptide and the Th peptide are reversed).

The present disclosure is also directed to compositions containing a Tau peptide immunogen construct. In some embodiments, the disclosed compositions contain more than one Tau peptide immunogen constructs to cover multiple B epitopes from Tau (e.g., 2, 3, 4 or more Tau peptide immunogen constructs). In certain embodiments, the compositions contain a mixture of Tau peptide immunogen constructs (e.g., any combination of two or more of SEQ ID NOs: 9-16, or any combination of two or more of SEQ ID Nos: 9-16 and 77-81) with more than one heterologous Th epitope derived from pathogenic proteins to cover a broad genetic background in patients. Compositions containing a mixture of peptide immunogen constructs can lead to a higher percentage in responder rate upon immunization for the treatment of tauopathies compared to compositions containing only a single Th peptide immunogen construct. In some embodiments, the peptide immunogen constructs targeting Tau target different regions of Tau (e.g., the N-terminal region of Tau for one peptide immunogen construct and the repeat domain for the second peptide immunogen construct; e.g., SEQ ID NOs: 10 and 15, respectively). In some embodiments, the N-terminal region of Tau targeted by one or more Tau peptide immunogen construct is within amino acids 1-44 of SEQ ID NO: 60. In some embodiments, the N-terminal region of Tau targeted by one or more Tau peptide immunogen construct is within amino acids 13-22 of SEQ ID NO: 60. In some embodiments, the region targeted by one or more Tau peptide immunogen construct is within amino acids 244-372 of SEQ ID NO: 60. In some embodiments, the region targeted by one or more Tau peptide immunogen construct is an amino acid region repeated (e.g., repeated 2, 3 or 4 times) within amino acids 244-372 of SEQ ID NO: 60. In some embodiments, the region targeted by one or more Tau peptide immunogen construct is within amino acids 250-320 of SEQ ID NO: 60. In some embodiments, the region targeted by one or more Tau peptide immunogen construct is an amino acid region repeated (e.g., repeated 2, 3 or 4 times) within amino acids 250-320 of SEQ ID NO: 60. In some embodiments, the region targeted by one or more Tau peptide immunogen construct is an amino acid region within 1R, 2R, 3R, and/or 4R repeat regions of Tau, which repeat regions are known in the art or described herein. In some embodiments, one or more peptide immunogen construct targets the region of Tau within amino acids 1-44 or 13-22 of SEQ ID NO:60, and one or more peptide immunogen construct targets the region of Tau within amino acids 244-372 or 250-320 of SEQ ID NO: 60 (or an amino acid region repeated within amino acids 244-372 or 250-320 of SEQ ID NO: 60 as described herein).

In some embodiments, the compositions comprising one or more Tau peptide immunogen constructs also includes one or more Aβ peptide immunogen construct(s) (e.g., a peptide immunogen construct comprising SEQ ID NO: 70, a peptide immunogen construct comprising SEQ ID NO: 71, or a combination of SEQ ID NOs: 70 and 71). In some embodiments, the compositions comprising one or more Tau peptide immunogen constructs also includes one or more Aβ peptide immunogen construct(s) (e.g., a peptide immunogen construct comprising SEQ ID NO: 70, a peptide immunogen construct comprising SEQ ID NO: 89, or a combination of SEQ ID NOs: 70 and 89).

The present disclosure is also directed to pharmaceutical compositions for the treatment and/or prevention of tauopathies. In some embodiments, the pharmaceutical compositions contain one, two or more of the disclosed peptide immunogen constructs in the form of a stabilized immunostimulatory complex formed through electrostatic associations by mixing a CpG oligomer (e.g., CpG1, CpG2, or CpG3) with a composition containing a peptide immunogen complex. Such stabilized immunostimulatory complexes are able to further enhance the immunogenicity of the peptide immunogen constructs. In some embodiments, the pharmaceutical compositions contain adjuvants such as mineral salts, including alum gel (ALHYDROGEL), aluminum phosphate (ADJUPHOS), or water-in-oil emulsions including MONTANIDE ISA 51 or 720.

The present disclosure is also directed to antibodies directed against the disclosed Tau peptide immunogen constructs. In particular, the Tau peptide immunogen constructs of the present disclosure are able to stimulate the generation of highly specific antibodies that are cross-reactive with the Tau amino acid sequences (e.g., any one or more of SEQ ID NOs: 1-8, 17-30, 63-67, or 202) when administered to a subject. The highly specific antibodies produced by the peptide immunogen constructs are cross reactive with recombinant Tau-containing proteins. The disclosed antibodies bind with high specificity to Tau without much, if any, directed to the heterologous Th epitopes employed for immunogenicity enhancement, which is in sharp contrast to the conventional protein or other biological carriers used for such peptide antigenicity enhancement.

The present disclosure is also directed to antibodies directed against the disclosed amyloid beta peptide immunogen constructs. In particular, the amyloid beta peptide immunogen constructs of the present disclosure are able to stimulate the generation of highly specific antibodies that are cross-reactive with the amyloid beta amino acid sequences (e.g., any one or more of SEQ ID NOs: 68, 69, or 83-88) when administered to a subject. The highly specific antibodies produced by the peptide immunogen constructs are cross reactive with recombinant amyloid beta-containing proteins. The disclosed antibodies bind with high specificity to amyloid beta without much, if any, directed to the heterologous Th epitopes employed for immunogenicity enhancement, which is in sharp contrast to the conventional protein or other biological carriers used for such peptide antigenicity enhancement.

The present disclosure also includes methods for treating and/or preventing tauopathies using the disclosed peptide immunogen constructs and/or antibodies directed against the peptide immunogen constructs. In some embodiments, the methods for treating and/or preventing tauopathies including administering to a subject (e.g., a mammal) a composition containing one, two or more disclosed peptide immunogen constructs. In certain embodiments, the compositions utilized in the methods contain any one or more of the disclosed peptide immunogen constructs in the form of a stable immunostimulatory complex with negatively charged oligonucleotides, such as CpG oligomers, through electrostatic association, which complexes are further supplemented, optionally, with a mineral salt or oil as adjuvant, for administration to patients with tauopathies. The disclosed methods also include dosing regimens, dosage forms, and routes for administering the peptide immunogen constructs to a host at risk for, or with, tauopathies. In some embodiments, the subject or patient is a human.

In some aspects, the disclosure provides a composition or a kit comprising a first Tau peptide immunogen construct comprising a first Tau B cell epitope comprising or consisting of about 6 to about 40 amino acid residues from the full-length Tau protein of SEQ ID NO: 60; and
(i) a second Tau peptide immunogen construct comprising a second Tau B cell epitope comprising or consisting of about 6 to about 40 amino acid residues from the full-length Tau protein of SEQ ID NO: 60, wherein the second B cell epitope is not the same as the first B cell epitope, and/or
(ii) an amyloid beta (Aβ) peptide immunogen construct comprising an amyloid beta B cell epitope comprising or consisting of about 6 to about 42 amino acid residues from the full-length amyloid beta protein of SEQ ID NO: 68.

In some embodiments of the foregoing or related aspects, the composition or kit disclosed herein comprises the second Tau peptide immunogen construct. In some embodiments of the foregoing or related aspects, the composition or kit disclosed herein comprises the amyloid beta peptide immunogen construct. In some embodiments of the foregoing or related aspects, the composition or kit disclosed herein comprises the second Tau peptide immunogen construct and the amyloid beta peptide immunogen construct.

In some embodiments of the foregoing or related aspects, the first Tau B cell epitope comprises or consists of about 6 to 40 amino acid residues from N-terminal amino acids 1 to 44 of SEQ ID NO: 60, and the second Tau B cell epitope comprises or consists of about 6 to 40 amino acid residues from repeat domain amino acids 244 to 372 of SEQ ID NO: 60. In some embodiments of the foregoing or related aspects, the amyloid beta B cell epitope comprises at least 6 amino acid residues from amino acids 1-16 of SEQ ID NO: 68. In some embodiments of the foregoing or related aspects, the amyloid beta B cell epitope comprises at least 8 amino acid residues from amino acids 1-16 of SEQ ID NO: 68.

In some embodiments of the foregoing or related aspects, the first Tau B cell epitope, the second Tau B cell epitope, and the amyloid beta B cell epitope are each or independently about 8 to 40 amino acids in length. In some embodiments of the foregoing or related aspects, the first Tau B cell epitope, the second Tau B cell epitope, and the amyloid beta B cell epitope are each or independently about 8 to 15 amino acids in length. In some embodiments of the foregoing or related aspects, the first Tau B cell epitope, the second Tau B cell epitope, and the amyloid beta B cell epitope are each or independently about 10 to 14 amino acids in length.

In some embodiments of the foregoing or related aspects, the first Tau B cell epitope and, if present, the second Tau B cell epitope are independently selected from SEQ ID NOs: 1-8, 17-30, 63-67, 72-76, 96, and 202. In some embodiments of the foregoing or related aspects, the first Tau B cell epitope comprises or consists of any one of SEQ ID NOs: 1-8, 17-30, 63-67, 72-76, 96, and 202, and the second Tau B cell epitope comprises or consists of any one of SEQ ID NOs: 1-8, 17-30, 63-67, 72-76, 96, and 202. In some embodiments, the first Tau B cell epitope and, if present, the second Tau B cell epitope are independently selected from SEQ ID NOs: 2, 4, 7, 72-76, and 202. In some embodiments of the foregoing or related aspects, the first Tau B cell epitope comprises or consists of SEQ ID NO: 2. In some embodiments of the foregoing or related aspects, the first Tau B cell epitope comprises or consists of SEQ ID NO: 7. In some embodiments of the foregoing or related aspects, the first Tau B cell epitope comprises or consists of SEQ ID NO: 202. In some embodiments of the foregoing or related aspects, the first Tau B cell epitope is selected from SEQ ID No: 1 or SEQ ID NO:2, and the second Tau B cell epitope is selected from SEQ ID NOs: 7 and 72-76. In some embodiments of the foregoing or related aspects, the first Tau B cell epitope comprises or consists of SEQ ID NO: 2, and the second Tau B cell epitope comprises or consists of SEQ ID NO: 7. In some embodiments of the foregoing or related aspects, the amyloid beta B cell epitope is selected from SEQ ID NOs: 69 and 82-88. In some embodiments of the foregoing or related aspects, the amyloid beta B cell epitope comprises or consists of SEQ ID NO: 69 or SEQ ID NO: 82.

In some embodiments of the foregoing or related aspects, the first Tau peptide immunogen construct comprises a first heterologous T helper (Th) epitope, the second Tau peptide immunogen construct, if present, comprises a second heterologous Th epitope, and the amyloid beta peptide immunogen construct, if present, comprises a third heterologous Th epitope. In some embodiments, the first heterologous Th epitope, the second heterologous Th epitope and the third heterologous Th epitope is each independently selected from SEQ ID NOs: 31-59 and 97. In some embodiments, the first heterologous Th epitope, the second heterologous Th epitope and the third heterologous Th epitope is each independently selected from SEQ ID NOs: 42, 44, 45 and 97. In some embodiments, each of the first heterologous Th epitope, the second heterologous Th epitope and the third heterologous Th epitope has SEQ ID NO: 44.

In some embodiments of the foregoing or related aspects, the first heterologous Th epitope and the first Tau B cell epitope, the second heterologous Th epitope and the second Tau B cell epitope, and the third heterologous Th epitope and the amyloid beta B cell epitope, are each covalently linked directly or through a heterologous spacer. In some embodiments, the composition or kit disclosed herein comprises the heterologous spacer linking the first heterologous Th epitope and the first Tau B cell epitope, the second heterologous Th epitope and the second Tau B cell epitope, and/or the third heterologous Th epitope and amyloid beta B cell epitope. In some embodiments, the heterologous spacer comprises one or more amino acids. In some embodiments, the one or more amino acids is Lys-, Gly-, Lys-Lys-Lys-, (α, ε-N)Lys, Lys-Lys-Lys-ε-Lys (SEQ ID NO: 95), or ε-Lys-Lys-Lys-Lys (SEQ ID NO: 62). In some embodiments, the one or more amino acids is Lys-Lys-Lys-ε-Lys (SEQ ID NO: 95) or ε-N-Lys-Lys-Lys-Lys (SEQ ID NO: 62).

In some embodiments of the foregoing or related aspects, the first Tau peptide immunogen construct and, if present, the second Tau peptide immunogen construct, is represented by the formulae:

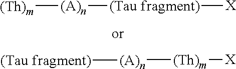

wherein
Th is a heterologous T helper epitope;
A is a heterologous spacer;
(Tau fragment) is a B cell epitope having about 6 to about 40 amino acid residues (optionally, about 10 to 40 amino acid residues) from the full-length Tau protein of SEQ ID NO: 60;
X is an α-COOH or α-CONH₂ of an amino acid;
m is from 1 to about 4; and
n is from 0 to about 10.

In some embodiments of the foregoing or related aspects, the amyloid beta peptide immunogen construct is represented by the formulae:

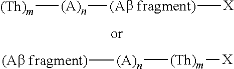

wherein
Th is a heterologous T helper epitope;
A is a heterologous spacer;
(Aβ fragment) is a B cell epitope having about 6 to about 42 amino acid residues (optionally, about 10 to 42 amino acid resides) from the full-length Aβ protein of SEQ ID NO: 68;
X is an α-COOH or α-CONH₂ of an amino acid;
m is from 1 to about 4; and
n is from 0 to about 10.

In some embodiments of the foregoing or related aspects, the first Tau peptide immunogen construct comprises the first heterologous Th epitope covalently linked to the carboxyl terminus of the first Tau B cell epitope, the second Tau peptide immunogen construct comprises the second heterologous Th epitope covalently linked to the amino terminus of the second Tau B cell epitope, and/or the amyloid beta peptide immunogen construct comprises the third heterologous Th epitope covalently linked to the carboxyl terminus of the amyloid beta B cell epitope.

In some embodiments of the foregoing or related aspects, the first Tau peptide immunogen construct and the second Tau peptide immunogen construct are selected from SEQ ID NOs: 9-16 and 77-81, or a construct in which the positions of the peptide cell epitope and the Th epitope are reversed. In some embodiments of the foregoing or related aspects, the first Tau peptide immunogen construct comprises or consists of the amino acid sequence of SEQ ID NO: 10 or SEQ ID NO: 15. In some embodiments of the foregoing or related aspects, the first Tau peptide immunogen construct comprises or consists of the amino acid sequence of SEQ ID NO: 10, and the second Tau peptide immunogen construct comprises or consists of the amino acid sequence of SEQ ID NO: 15. In some embodiments of the foregoing or related aspects, the first Tau peptide immunogen construct and/or the second Tau peptide immunogen construct is selected from SEQ ID NOs: 77-81, optionally wherein the first Tau peptide immunogen construct has the amino acid sequence of SEQ ID NO: 10 and the second Tau peptide immunogen construct is selected from SEQ ID NOs: 77-81. In some embodiments of the foregoing or related aspects, the amyloid beta peptide immunogen construct is selected from SEQ ID Nos: 70, 71 and 89, or a construct in which the positions of the B cell epitope and the Th epitope are reversed. In some embodiments, the amyloid beta peptide immunogen construct comprises or consists of SEQ ID NO: 70. In some embodiments, the amyloid beta peptide immunogen construct comprises or consists of SEQ ID NO: 89.

In some aspects, the disclosure provides a Tau peptide immunogen construct comprising a B cell epitope comprising an amino acid sequence of SEQ ID NO: 202.

In some aspects, the disclosure provides a Tau peptide immunogen construct comprising a B cell epitope comprising an amino acid sequence selected from SEQ ID NOs: 72-76. In some embodiments of the foregoing or related aspects, the Tau peptide immunogen construct disclosed herein is represented by the formulae:

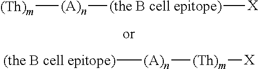

wherein
Th is a heterologous T helper epitope;
A is a heterologous spacer;
(the B cell epitope) comprises, essentially consists of, or consists of an amino acid sequence selected from any one of SEQ ID NOs: 72-76;
X is an α-COOH or α-CONH₂ of an amino acid;
m is from 1 to about 4; and
n is from 0 to about 10.

In some embodiments, the Tau peptide immunogen construct disclosed herein comprises, essentially consists of, or consists of the amino acid sequence of any one of SEQ ID NOs: 77-81.

In some aspects, the disclosure provides an amyloid beta peptide immunogen construct comprising a B cell epitope comprising, essentially consisting of, or consisting of an amino acid sequence of SEQ ID NO: 82. In some embodiments of the foregoing or related aspects, the amyloid beta peptide immunogen construct disclosed herein is represented by the formulae:

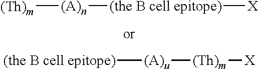

wherein
Th is a heterologous T helper epitope;
A is a heterologous spacer;
(the B cell epitope) comprises, essentially consists of, or consists of the amino acid sequence of SEQ ID NO: 82;
X is an α-COOH or α-CONH₂ of an amino acid;
m is from 1 to about 4; and
n is from 0 to about 10.

In some embodiments, the amyloid beta peptide immunogen construct disclosed herein comprises, essentially consists of, or consists of the amino acid sequence of SEQ ID NO: 89.

In some aspects, the disclosure provides a composition or kit comprising a Tau peptide immunogen construct disclosed herein.

In some aspects, the disclosure provides a composition or kit comprising an amyloid beta peptide immunogen construct disclosed herein.

In some embodiments of the foregoing or related aspects, the composition or kit disclosed herein is a single composition comprising the first Tau peptide immunogen construct, and at least one of (or both of): (i) the second Tau peptide immunogen construct and (ii) the amyloid beta peptide immunogen construct. In other embodiments, a composition comprising the first Tau peptide immunogen construct can be combined or co-administered with one or more compositions comprising one or more of: (i) the second Tau peptide immunogen construct and (ii) the amyloid beta peptide immunogen construct.

In some embodiments of the foregoing or related aspects, the composition or kit disclosed herein is a kit comprising the first Tau peptide immunogen construct, and at least one of: the second Tau peptide immunogen construct and the amyloid beta peptide immunogen construct. In some embodiments, prior to administration to a subject, the components of the kit are mixed to create a single composition comprising the first Tau peptide immunogen construct, and at least one of: the second Tau peptide immunogen construct and the amyloid beta peptide immunogen construct. In some embodiments, the components of the kit are used to create a first composition comprising the first Tau peptide immunogen construct, and a second composition comprising at least one of: the second Tau peptide immunogen construct and the amyloid beta peptide immunogen construct.

In some aspects, the disclosure provides a pharmaceutical composition comprising the composition disclosed herein, and a pharmaceutically acceptable delivery vehicle and/or adjuvant. In some embodiments of the foregoing or related aspects, the first Tau peptide immunogen construct, the second Tau peptide immunogen construct and/or the amyloid beta peptide immunogen construct are mixed with an CpG oligodeoxynucleotide (ODN) to form a stabilized immunostimulatory complex. In some embodiments, the pharmaceutical composition disclosed herein comprises 3 or 4 of Tau peptide immunogen constructs. In some embodiments, the pharmaceutical composition disclosed herein comprises more than one amyloid beta peptide immunogen constructs.

In some aspects, the disclosure provides an isolated antibody or epitope-binding fragment thereof that specifically binds to the B cell epitope of the Tau peptide immunogen construct or the amyloid beta peptide immunogen construct obtained by administering to a mammal a composition or compositions disclosed herein, or a peptide immunogen construct disclosed herein. In some embodiments of the foregoing or related aspects, the isolated antibody or epitope-binding fragment thereof disclosed herein is bound to the Tau peptide immunogen construct or the amyloid beta peptide immunogen construct.

In some aspects, the disclosure provides a composition comprising an isolated antibody or epitope-binding fragment thereof disclosed herein.

In some aspects, the disclosure provides a method of preventing, inhibiting, reducing the severity of, delaying, or treating a tauopathy in a subject, the method comprising administering to the subject a Tau peptide immunogen construct, an amyloid beta peptide immunogen construct, a composition or compositions disclosed herein. In some embodiments of the foregoing or related aspects, the method comprises administering the composition wherein the first Tau peptide immunogen construct and at least one of the second Tau peptide immunogen construct and the amyloid beta peptide immunogen construct are comprised within a single composition.

In some aspects, the disclosure provides a method of preventing, inhibiting, reducing the severity of, delaying, or treating a tauopathy in a subject, the method comprising administering to the subject: a first Tau peptide immunogen construct comprising a first Tau B cell epitope comprising or consisting of about 6 to about 40 (e.g., 6 to 40, 8 to 40, or 8 to 15) amino acid residues from the full-length Tau protein of SEQ ID NO: 60; and
(i) a second Tau peptide immunogen construct comprising a second Tau B cell epitope comprising or consisting of about 6 to about 40 (e.g., 6 to 40, 8 to 40, or 8 to 15) amino acid residues from the full-length Tau protein of SEQ ID NO: 60, wherein the second B cell epitope is not the same as the first B cell epitope, and/or
(ii) an amyloid beta (Aβ) peptide immunogen construct comprising an amyloid beta B cell epitope comprising or consisting of about 6 to about 42 (e.g., 6 to 40, 8 to 40, or 8 to 15) amino acid residues from the full-length amyloid beta protein of SEQ ID NO: 68; and
   wherein the first Tau peptide immunogen construct, and the second Tau peptide immunogen construct and/or the amyloid beta peptide immunogen construct, are comprised within separate compositions, which are administered separately. In other embodiments of the method, the first Tau peptide immunogen construct, and the second Tau peptide immunogen construct and/or the amyloid beta peptide immunogen construct, are comprised within a single composition.

In some embodiments of the foregoing or related aspects, the tauopathy is selected from Alzheimer's disease, Lewy body disease, frontotemporal dementia, parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, dementia pugilistica, Down syndrome, Gerstmann-Straussler-Scheinker disease, Hallerworden-Spatz disease, inclusion body myositis, Creutzfeld-Jakob disease, multiple system atropy, Niemann-Pick disease type C, prion protein cerebral amyloid angiopathy, subacute sclerosing panencephalitis, myotonic dystrophy, non-guanamian motor neuron disease with neurofibrillary tangles, postencephalitic parkinsonism, and chronic traumatic encephalopathy. In some embodiments, the tauopathy is Alzheimer's disease. In some embodiments, the subject is a mammal. In some embodiments, the subject is a human.

### BRIEF DESCRIPTION OF THE DRAWINGS

FIG. 1 is a schematic diagram showing a Th peptide carrier linked to a target-specific B-cell epitope (Top) and tau primary structure with functional domains and targeting peptides annotated (Bottom).

FIGS. 2A and 2B are a dot blot, set of graphs, and table showing the results of antibody binding characterization.

FIGS. 3A and 3B are a set of graphs showing the results of an aggregation assay using a Tau biosensor line.

FIGS. 4A and 4B are a set of graphs showing the results of a Tau uptake assay.

FIGS. 5A and 5B are graphs showing the results of a Tau uptake assay. FIG. 5C is a graph showing biolayer interferometry analysis of IgGs from p5555kb, IgGs from p5187kb, and mixed IgGs from p5555kb and p5187kb. FIG. 5D shows anti-tau IgG isotype EC50 titers.

FIGS. 6A-6C are graphs and photographic images showing the results of vaccination of WT and P301L mice with p5555kb. The arrowheads in FIG. 6A indicate the injections.

FIGS. 7A-7E are graphs showing IgG titers in rats immunized with p5555kb, p5187kb, or VXX-301, VXX-301 refers to the combination vaccine of p5555kb and p5187kb in one formulation. FIGS. 7A-7B show IgG titers against monomeric Tau (FIG. 7A) or fibril Tau (FIG. 7B) following vaccination with p5555kb, p5187kb, or VXX-301. FIGS. 7C-7E show IgG titer curves over time against monomeric Tau following vaccination with p5555kb (FIG. 7C), p5187kb (FIG. 7D), or VXX-301 (FIG. 7E).

FIG. 8 is a graph showing biolayer interferometry analysis of IgGs from p5555kb, p5187kb, or VXX-301. IgG1 and IgG2b were enriched following vaccination with lower titers for IgG2a and IgG2c.

FIG. 9 is a graph showing results of Western blot analysis of anti-Tau antibodies from rats vaccinated with p5555kb, p5187kb or VXX-301. Different recombinant preparations of Tau were probed for binding, including full length preparations 2N4R, 2N3R, WT preformed fibrils (PFF), P301S, and P301L K18 (C-terminal fragment of Tau containing the 4 repeat domains). White arrow indicates p5187kb and VXX-301 derived antibodies could bind to all Tau preparations.

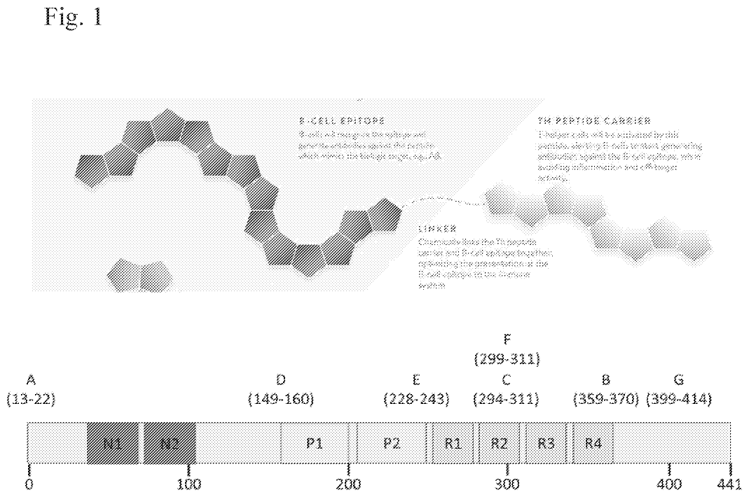

FIGS. 1A-10B are graphs showing the inhibition of Tau aggregation by anti-Tau mAb Bepranemab or anti-Tau antibodies from rats vaccinated with p5555kb, p5187kb, or VXX-301.

FIGS. 11A-11B are graphs showing the results of a pHrodo-PFF Tau uptake assay in b103 cells with anti-Tau mAbs Semorinemab (Sem), Bepranemab (Bep), or anti-Tau antibodies from rats vaccinated with p5555kb, p5187kb, or VXX-301.

FIGS. 12A-12D are graphs showing protection against Tau-associated pathology and death, and antibody titers in female P301L mice vaccinated with saline control, p5555kb, p6187kb, or VXX-301. FIGS. 12A-12B show Kaplan Meier curves of observed onset of ataxia (FIG. 12A) and survival of P301L mice (FIG. 12B). FIGS. 12C-12D show terminal anti-Tau antibody titers against monomeric Tau (FIG. 12C) or fibril Tau (FIG. 12D). The arrowheads in FIGS. 12A-12B indicate the injections.

FIGS. 13A and 13B are graphs showing the immunogenicity of Aβ (p3102kb) and Tau (p5555kb, p5404kb, or p5137kb) combination vaccines against monomeric Tau, fibril Tau, fibril P301S Tau, or a control B-cell epitope peptide not conjugated to a Th peptide (α-peptide).

### DETAILED DESCRIPTION OF THE INVENTION

The present disclosure is directed to individual peptide immunogen constructs targeting portions of the Tau protein for the treatment and/or prevention of Alzheimer's disease and tauopathies. The present disclosure is also directed to compositions containing the Tau peptide immunogen constructs, methods of making and using the peptide immunogen constructs, and antibodies produced by the peptide immunogen constructs. The present disclosure is further directed to compositions comprising more than one (e.g., 2, 3, or 4) Tau peptide immunogen constructs (e.g., one or more of SEQ ID NOs: 9-16; e.g., 10 and 15; or one or more of SEQ ID NO: 77-81; in any combination; also see below), and methods of using such compositions or combinations for the prevention and/or treatment of Alzheimer's disease and tauopathies. The present disclosure further provides compositions comprising one or more (e.g., 1, 2, 3, or 4) Tau peptide immunogen construct and one or more (e.g., 1, 2, 3, or 4) Aβ peptide immunogen construct (e.g., SEQ ID NO: 70, SEQ ID NO: 71, SEQ ID NO: 89, or any combinations thereof such as SEQ ID NO: 70 and SEQ ID NO: 71, or SEQ ID NO: 70 and SEQ ID NO: 89), and methods of using such compositions or combinations for the prevention and/or treatment of Alzheimer's disease and tauopathies. In some embodiments, SEQ ID NO: 10 is combined with SEQ ID NOs: 70 and 71. In some embodiments, SEQ ID NO: 11 is combined with SEQ ID NOs: 70 and 71. In some embodiments, SEQ ID NO: 12 is combined with SEQ ID NOs: 70 and 71. In some embodiments, SEQ ID NO: 13 is combined with SEQ ID NOs: 70 and 71. In some embodiments, SEQ ID NO: 14 is combined with SEQ ID NOs: 70 and 71. In some embodiments, SEQ ID NO: 15 is combined with SEQ ID NOs: 70 and 71. In some embodiments, SEQ ID NO: 16 is combined with SEQ ID NOs: 70 and 71. In some embodiments, an additional, different sequence of any one of SEQ ID NOs: 9, 10, 11, 12, 13, 14, 15, or 16 is added to an above-listed combination (such as any additional Tau peptide immunogen construct sequence described herein). In some embodiments, only SEQ ID NO: 70 or only SEQ ID NO: 71 is included in an above-listed combination. In some embodiments, SEQ ID NO: 10 is combined with SEQ ID NOs: 70 and/or 89. In some embodiments, SEQ ID NO: 11 is combined with SEQ ID NOs: 70 and/or 89. In some embodiments, SEQ ID NO: 12 is combined with SEQ ID NOs: 70 and/or 89. In some embodiments, SEQ ID NO: 13 is combined with SEQ ID NOs: 70 and/or 89. In some embodiments, SEQ ID NO: 14 is combined with SEQ ID NOs: 70 and/or 89. In some embodiments, SEQ ID NO: 15 is combined with SEQ ID NOs: 70 and/or 89. In some embodiments, SEQ ID NO: 16 is combined with SEQ ID NOs: 70 and/or 89. In some embodiments, SEQ ID NO: 77 is combined with SEQ ID NOs: 70 and/or 89. In some embodiments, SEQ ID NO: 78 is combined with SEQ ID NOs: 70 and/or 89. In some embodiments, SEQ ID NO: 79 is combined with SEQ ID NOs: 70 and/or 89. In some embodiments, SEQ ID NO: 80 is combined with SEQ ID NOs: 70 and/or 89. In some embodiments, SEQ ID NO: 81 is combined with SEQ ID NOs: 70 and/or 89. In some embodiments, an additional, different Tau peptide immunogen construct sequence is added to any of the above-listed combinations (such as any additional Tau peptide immunogen construct sequence described herein). In some embodiments, only SEQ ID NO: 70 or only SEQ ID NO: 89 is included in any of the above-listed combinations.

The disclosed peptide immunogen constructs contain about 15 or more amino acids. In some embodiments, the disclosed peptide immunogen constructs contain about 80 or less, about 75 or less, about 70 or less, about 60 or less, about 50 or less, about 40 or less, or about 35 or less amino acids.

In some embodiments, the Tau peptide immunogen constructs contain a B cell epitope from portions of the longest isoform of the human Tau protein (GenBank: AGF19246.1) having the amino acid sequence shown in Table 6. For example, Tau B cell epitopes provided in Tables 1 and 7-10 can be used in the peptide immunogen constructs described herein. In some embodiments, the amyloid beta peptide immunogen constructs contain a B cell epitope from a portion of Aβ (see, e.g., Table 3). For example, amyloid beta B cell epitopes provided in Table 3, WO 2014/143087 or U.S. Pat. No. 9,102,752 B2 (each of which is incorporated by reference herein) can be used in the peptide immunogen constructs described herein. The B cell epitopes can each be linked to a heterologous T helper cell (Th) epitope derived from a pathogen protein through an optional heterologous spacer. The disclosed peptide immunogen constructs stimulate the generation of highly specific antibodies directed against Tau or Aβ. The disclosed peptide immunogen constructs can be used as an immunotherapy for patients suffering from Alzheimer's disease and/or tauopathies.

The B cell epitope portion of the Tau peptide immunogen constructs can have amino acid sequences from the full-length Tau protein (Table 6). In some embodiments, the B cell epitope has a sequence containing any one of the target sequences listed in Table 1 (e.g., SEQ ID NOs: 1-8, 202). In some embodiments, the Tau B cell epitope has a sequence provided in any one of Tables 7-10. In some embodiments, the Tau B cell epitope has any immunogenic sequence provided in any one of Tables 1 and 7-10 (a sequence capable of eliciting anti-Tau antibodies). In some embodiments, the Tau B cell epitope has a sequence of p5543a, p5546a, p5547a, p5555a, p5557a, p5558a, p5559a, p5564a, p5565a, p5566a, p5569a, p5695a, p5696, or p5535a, which are SEQ ID NOs: 17-30, respectively; or a sequence of p5556a, p5456a, p4579a, p5541a, or p4062a, which are SEQ ID NOs: 63-67, respectively, also shown in Table 1.

The B cell epitope portion of the Aβ peptide immunogen constructs can have amino acid sequences from Aβ₁₋₄₂, e.g., Aβ₁₋₁₄ (see Table 3; also see additional Aβ peptides in WO 2014/143087).

In some embodiments, the B cell epitope portion of the Aβ peptide immunogen construct has any one amino acid sequence shown in Table 3. In some embodiments, the B cell epitope portion of the Aβ peptide immunogen construct has any one Aβ peptides amino acid sequence in WO 2014/143087 and/or U.S. Pat. No. 9,102,752 B2, each of which is incorporated by reference herein in its entirety. The disclosure of amyloid beta B cell epitopes, and amyloid beta peptide immunogen constructs comprising such B cell epitopes, in WO 2014/143087 and/or U.S. Pat. No. 9,102,752 B2, is specifically incorporated by reference herein. In some embodiments, the Aβ B cell epitope has any Aβ B cell epitope sequence provided in WO 2014/143087 and/or U.S. Pat. No. 9,102,752 B2. In some embodiments, the Aβ B cell epitope has any immunogenic Aβ B cell epitope sequence provided in WO 2014/143087 and/or U.S. Pat. No. 9,102,752 B2 (a sequence capable of eliciting anti-Aβ antibodies).

The Tau and Aβ peptide immunogen constructs of the present disclosure can contain a heterologous Th epitope amino acid sequence derived from a pathogenic protein (e.g., any one of SEQ ID NOs: 31 to 59, and 97) as shown in Table 5. In certain embodiments, the heterologous Th epitope is derived from natural pathogens, such as Diphtheria Toxin (SEQ ID NO: 35), Plasmodium Falciparum (SEQ ID NO: 36), Cholera Toxin (SEQ ID NO: 38). In other embodiments, the heterologous Th epitope is an idealized artificial Th epitope derived from Measles Virus Fusion protein (MVF 1 to 5) or Hepatitis B Surface Antigen (HBsAg 1 to 3) in the form of either single sequence or combinatorial sequences (e.g., any one of SEQ ID NOs: 32, 39, or 41-45). In some embodiments, the heterologous Th epitope has amino acid sequence selected from SEQ ID NO: 42, 44, 45, and 97. In some embodiments, the heterologous Th epitope has amino acid sequence of SEQ ID NO:44.

In some embodiments, the peptide immunogen constructs contain a B cell epitope from Tau or Aβ linked to a heterologous T helper cell (Th) epitope through an optional heterologous spacer. In certain embodiments, the peptide immunogen constructs contain a B cell antigenic site having an amino acid sequence from Tau (see Table 1 (e.g., any one of SEQ ID NOs: 1-8) and Tables 7-10 (e.g., any one of SEQ ID NOs: 17-30 or 63-67) or from Aβ (e.g., SEQ ID NO: 68 or 69) linked to a heterologous Th epitope derived from a pathogenic protein (e.g., any one of SEQ ID NOs: 31 to 59) through an optional heterologous spacer. In some embodiments, the optional heterologous spacer is a molecule or chemical structure capable of linking two amino acids and/or peptides together. In certain embodiments, the spacer is a naturally occurring amino acid, a non-naturally occurring amino acid, or a combination thereof. In specific embodiments, the Tau peptide immunogen constructs have an amino acid sequence shown in Table 2 (e.g., a sequence of any one of SEQ ID NOs: 9-16 or a construct in which the positions of the Tau peptides and the Th epitopes are reversed). In specific embodiments, the Tau peptide immunogen constructs have an amino acid sequence of any one of SEQ ID NOs: 77-81 or a construct in which the positions of the Tau peptides and the Th epitopes are reversed. In specific embodiments, the Aβ peptide immunogen constructs have an amino acid sequence shown in Table 4 (e.g., a sequence of SEQ ID NO: 70, 71, or 89, or a construct in which the positions of the Aβ peptide and the Th epitopes are reversed).

The present disclosure is also directed to compositions containing a Tau peptide immunogen construct. In some embodiments, the disclosed compositions contain more than one Tau peptide immunogen construct to cover multiple B epitopes from Tau. In certain embodiments, the compositions contain a mixture of Tau peptide immunogen constructs with more than one heterologous Th epitope derived from pathogenic protein to cover a broad genetic background in patients. Compositions containing a mixture of peptide immunogen constructs can lead to a higher percentage in responder rate upon immunization for the treatment of Alzheimer's disease and/or tauopathies compared to compositions containing only a single peptide immunogen construct. The present disclosure is also directed to compositions containing one or more Tau peptide immunogen construct, e.g., as described herein, and one or more Aβ peptide immunogen construct, e.g., as described herein.

The present disclosure is also directed to pharmaceutical compositions for the treatment and/or prevention of Alzheimer's disease and/or tauopathies. In some embodiments, the pharmaceutical compositions contain the disclosed peptide immunogen constructs or combinations thereof (e.g., mixtures of Tau peptide immunogen constructs or mixtures of Tau peptide immunogen constructs and Aβ peptide immunogen constructs) in the form of a stabilized immunostimulatory complex formed through electrostatic associations by mixing a CpG oligomer with a composition containing a peptide immunogen complex. Such stabilized immunostimulatory complexes are able to further enhance the immunogenicity of the peptide immunogen constructs. In some embodiments, the pharmaceutical compositions contain adjuvants such as mineral salts, including alum gel (ALHYDROGEL), aluminum phosphate (ADJUPHOS), or water-in-oil emulsions including MONTANIDE ISA 51 or 720. The peptide immunogen constructs described herein, when used in combination, can be comprised within a single composition, or can be comprised within separate compositions.

The present disclosure is also directed to antibodies directed against the disclosed Tau peptide immunogen constructs and/or disclosed amyloid beta peptide immunogen constructs. In particular, the peptide immunogen constructs of the present disclosure are able to stimulate the generation of highly specific antibodies that are cross-reactive with the target amino acid sequences when administered to a subject. For example, the highly specific antibodies produced by the peptide immunogen constructs are cross reactive with recombinant Tau-containing proteins. The disclosed antibodies bind with high specificity to Tau without much, if any, directed to the heterologous Th epitopes employed for immunogenicity enhancement, which is in sharp contrast to the conventional protein or other biological carriers used for such peptide antigenicity enhancement.

The present disclosure also includes methods for treating and/or preventing Alzheimer's disease and/or tauopathies using the disclosed peptide immunogen constructs (including, e.g., the combinations described above and elsewhere herein) and/or antibodies directed against the peptide immunogen constructs. In some embodiments, the methods for treating and/or preventing Alzheimer's disease and/or tauopathies including administering to a host a composition containing a disclosed peptide immunogen construct. In certain embodiments, the compositions utilized in the methods contain one or more disclosed peptide immunogen construct in the form of a stable immunostimulatory complex with negatively charged oligonucleotides, such as CpG oligomers, through electrostatic association, which complexes are further supplemented, optionally, with a mineral salt or oil as adjuvant, for administration to patients with Alzheimer's disease and/or tauopathies. The disclosed methods also include dosing regimens, dosage forms, and routes for administering the peptide immunogen constructs to a host at risk for, or with, Alzheimer's disease and/or tauopathies.

The section headings used herein are for organizational purposes only and are not to be construed as limiting the subject matter described. All references or portions of references cited in this application are expressly incorporated by reference herein in their entirety for any purpose.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all amino acid sizes, and all molecular weight or molecular mass values, given for polypeptides are approximate, and are provided for description. Although methods and materials similar or equivalent to those described herein can be used in the practice or testing of the disclosed method, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Terminology

The term “about,” as used herein, refers to a range of +/−10% of the stated value. In some embodiments, the range is +/−5%, +/−3%, +/−2%, +/−1%, +/−0.5%, or +/−0.1% of the stated value.

The term “peptide immunogen” or “peptide immunogen construct,” as used herein, refers to peptides comprising a heterologous T helper cell epitope covalently linked to a peptide containing a B cell epitope. The covalent linkage may be through a covalent peptide bond, thioester or a heterologous spacer. In some embodiments, the heterologous spacer is or comprises an amino acid sequence.

The term “artificial,” as used herein in connection with the peptide immunogen or Th epitopes, refers to amino acid sequences that are not naturally occurring.

The term “promiscuous,” as used herein in connection with Th epitopes, refers to Th epitopes that are reactive across species and across individuals of a single species.

The term “preventing” a disease or condition in a subject, as used herein, refers to administering a medicament (e.g., the peptide immunogen construct described herein) to the subject prior to the onset of the disease or condition, when administration of the medicament to a statistical sample prior to the onset of the disease or condition reduces the occurrence of the disease or condition in the treated sample relative to an untreated control sample, or delays the onset or reduces the occurrence or severity of one or more symptoms of the disease or condition relative to the untreated control sample.

The term “treating” a disease or condition in a subject, as used herein, refers to administering a medicament (e.g., the peptide immunogen construct described herein) to the subject having or suspected of having a disease or condition (i.e., after the onset of the disease or condition), such that at least one symptom of the disease or condition is decreased or prevented from worsening.

### Peptide Immunogen Constructs

The present disclosure provides peptide immunogen constructs containing a B cell epitope with an amino acid sequence from Tau or amyloid beta (Aβ) covalently linked to a heterologous T helper cell (Th) epitope directly or through an optional heterologous spacer.

In some embodiments, the peptide immunogen constructs described herein are modified as described herein, e.g., by acetylation of amino acids, N-methylation of amino acids, substitution of one or two amino acids with cysteine for purposes of disulfide bond formation, and/or use of N-terminal pyroglutamic acid. Acceptable modifications are any described herein (see, e.g., Table 2 and Table 4). In some embodiments, the modifications contemplated herein protein the peptide immunogen construct from protease degradation.

The phrase “Tau peptide immunogen construct,” as used herein, refers to a peptide containing (a) a B cell epitope having about 6 or more amino acid residues from the full-length sequence of the longest Tau isoform (SEQ ID NO: 60) (e.g., about 6-40, 8-40, 9-30, 10-25, 8-20, 9-18, 8-15, 10-15, 8-14, or 10-14; or about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids); (b) a heterologous Th epitope; and (c) an optional heterologous spacer.

In certain embodiments, the Tau peptide immunogen construct can be represented by the formulae:

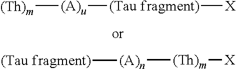

wherein
Th is a heterologous T helper epitope;
A is a heterologous spacer;
(Tau fragment) is a B cell epitope having about 6 to about 40 amino acid residues from SEQ ID NO: 60 (e.g., about 6-40, 8-40, 9-30, 10-25, 8-20, 9-18, 8-15, 10-15, 8-14, or 10-14; or about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids);
X is an α-COOH or α-CONH₂ of an amino acid;
m is from 1 to about 4; and
n is from 0 to about 10.

In some embodiments, the Tau peptide immunogen construct is selected from any one of SEQ ID NOs: 9-16, and 77-81 or any combination of two, three or more thereof is used (see Table 2). In some embodiments, the Tau peptide immunogen construct is selected from any one of SEQ ID NOs: 9-16, or any combination of two, three or more thereof is used (see Table 2). In some embodiments, the Tau peptide immunogen construct is selected from any one of SEQ ID NOs: 77-81, or any combination of two, three or more thereof is used (see Table 2).

In some embodiments, more than one (e.g., 2, 3, 4 or more) Tau peptide immunogen construct is used (e.g., in compositions, kits and methods described herein) to elicit antibodies targeting multiple B epitopes from Tau (e.g., 2, 3, 4 or more epitopes, respectively). In some embodiments, the more than one Tau peptide immunogen constructs comprise B cell epitope from different regions of Tau (e.g., the N-terminal region of Tau for one peptide immunogen construct and the repeat domain for the second peptide immunogen construct; e.g., SEQ ID NOs: 10 and 15, respectively). In some embodiments, the N-terminal region of Tau is within amino acids 1-44 (e.g., amino acids 13-22) of SEQ ID NO: 60. In some embodiments, the repeat domain of Tau is within amino acids 244-372 of SEQ ID NO: 60. In some embodiments, a Tau peptide immunogen construct comprising a B cell epitope within amino acids 1-44 (e.g., amino acids 13-22) of SEQ ID NO: 60 is used together with (e.g., in the same composition, same kit, or co-administered with) another Tau peptide immunogen construct comprising a B cell epitope within amino acids 244-372 of SEQ ID NO: 60. In some embodiments, one of the Tau peptide immunogen constructs comprises a B cell epitope from the N-terminal region of Tau (having a sequence from within amino acids 1-44, e.g., amino acids 13-22) of SEQ ID NO: 60. In some embodiments, another Tau peptide immunogen constructs comprises a B cell epitope from an amino acid region that is repeated (e.g., repeated 2, 3 or 4 times) within amino acids 244-372 of SEQ ID NO: 60. In some embodiments, the amino acid region that is repeated is an amino acid region within 1R, 2R, 3R, and/or 4R repeat regions of Tau, which repeat regions are known in the art or described herein. In some embodiments, one Tau peptide immunogen construct comprises a B cell epitope from the N-terminal region, and one Tau peptide immunogen construct comprises a B cell epitope from a region other than the N-terminal region (e.g., where one or both of such constructs elicit antibodies binding to 4, 5, or each of 6 isoforms of Tau provided in Table 6). In some embodiments, one Tau peptide immunogen construct comprises a B cell epitope from the repeat domain on Tau, and one Tau peptide immunogen construct comprises a B cell epitope from a region other than the repeat domain of Tau (e.g., where one or both of such constructs elicit antibodies binding to 4, 5, or each of 6 isoforms of Tau provided in Table 6).

In some embodiments, at least one Tau peptide immunogen construct selected from any one of SEQ ID NOs: 9 and 10 is used together with (e.g., in the same composition, kit, or co-administered with) at least one Tau peptide immunogen construct selected from any one of SEQ ID NOs: 15, and 77-81.

In some embodiments, at least one Tau peptide immunogen construct selected from any one of SEQ ID NOs: 9-16 is used together with (e.g., in the same composition, kit, or co-administered with) at least one Tau peptide immunogen construct selected from any one of SEQ ID NOs: 77-81.

In some embodiments, at least one Tau peptide immunogen construct of SEQ ID NOs: 10 is used together with (e.g., in the same composition, kit, or co-administered with) at least one Tau peptide immunogen construct selected from any other provided herein.

In some embodiments, at least one Tau peptide immunogen construct selected from any one of SEQ ID NOs: 15 and 77-81 is used together with (e.g., in the same composition, kit, or co-administered with) at least one Tau peptide immunogen construct selected from any other provided herein.

In some embodiments, at least one Tau peptide immunogen construct of SEQ ID NOs: 10 is used together with (e.g., in the same composition, kit, or co-administered with) at least one Tau peptide immunogen construct selected from any one of SEQ ID NOs: 15 and 77-81.

In some embodiments, a composition or a kit comprises at least one Tau peptide immunogen construct comprising or consisting of SEQ ID NO: 10, and at least one Tau peptide immunogen construct comprising or consisting of SEQ ID NO: 15 (or a modified version thereof comprising modified amino acids or chemical groups). Acceptable modifications are any described herein (such as in SEQ ID NOs:77-81). In some embodiments, a composition or a kit comprises Tau peptide immunogen construct of SEQ ID NO: 10, and Tau peptide immunogen construct of SEQ ID NO: 15. In some embodiments, a composition or a kit comprises Tau peptide immunogen construct of SEQ ID NO: 10, and at least one Tau peptide immunogen construct of any one of SEQ ID NOs: 77-81.

In some embodiments, the Tau peptide immunogen construct comprises, essentially consists of, or consists of SEQ ID NO:9. In some embodiments, the Tau peptide immunogen construct comprises, essentially consists of, or consists of SEQ ID NO:10. In some embodiments, the Tau peptide immunogen construct comprises, essentially consists of, or consists of SEQ ID NO:11. In some embodiments, the Tau peptide immunogen construct comprises, essentially consists of, or consists of SEQ ID NO:12. In some embodiments, the Tau peptide immunogen construct comprises, essentially consists of, or consists of SEQ ID NO:13. In some embodiments, the Tau peptide immunogen construct comprises, essentially consists of, or consists of SEQ ID NO:14. In some embodiments, the Tau peptide immunogen construct comprises, essentially consists of, or consists of SEQ ID NO:15. In some embodiments, the Tau peptide immunogen construct comprises, essentially consists of, or consists of SEQ ID NO:16. In some embodiments, the Tau peptide immunogen construct comprises, essentially consists of, or consists of SEQ ID NO:77. In some embodiments, the Tau peptide immunogen construct comprises, essentially consists of, or consists of SEQ ID NO:78. In some embodiments, the Tau peptide immunogen construct comprises, essentially consists of, or consists of SEQ ID NO:79. In some embodiments, the Tau peptide immunogen construct comprises, essentially consists of, or consists of SEQ ID NO:80. In some embodiments, the Tau peptide immunogen construct comprises, essentially consists of, or consists of SEQ ID NO:81.

The phrase “Amyloid beta or Aβ peptide immunogen construct,” as used herein, refers to a peptide containing (a) a B cell epitope having about 6 or more amino acid residues from the full-length sequence of amyloid beta protein of SEQ ID NO: 68 (e.g., about 6-40, 8-40, 9-30, 10-25, 8-20, 9-18, 8-15, 10-15, 8-14, or 10-14; or about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids); (b) a heterologous Th epitope; and (c) an optional heterologous spacer.

In certain embodiments, the Aβ peptide immunogen construct can be represented by the formulae:

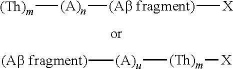

wherein
Th is a heterologous T helper epitope;
A is a heterologous spacer;
(Aβ fragment) is a B cell epitope having about 6 to about 40 amino acid residues from SEQ ID NO: 68 (e.g., about 6-40, 8-40, 9-30, 10-25, 8-20, 9-18, 8-15, 10-15, 8-14, or 10-14; or about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids);
X is an α-COOH or α-CONH₂ of an amino acid;
m is from 1 to about 4; and
n is from 0 to about 10.

In some embodiments, the Aβ peptide immunogen construct is selected from SEQ ID NO: 70 and SEQ ID NO: 71 or a combination thereof is used (see Table 4). In some embodiments, the Aβ peptide immunogen construct is selected from SEQ ID NO: 70, SEQ ID NO: 71 and SEQ ID NO:89, or any combination of two or three thereof is used (see Table 4).

Also see WO 2014/143087 for additional Aβ peptides and Aβ peptide immunogenic constructs that can be used in combination with Tau peptide immunogen constructs described herein. Peptides immunogen constructs falling within the formulae disclosed in WO 2014/143087 can also be used and are included in this disclosure. In some embodiments, the Aβ peptide immunogen construct can be represented by any of the Aβ peptide immunogen construct formulas presented in WO 2014/143087 and/or U.S. Pat. No. 9,102,752 B2, the disclosures of which are specifically incorporated by reference herein in respect to such formulas.

In some embodiments, the Aβ peptide immunogen construct comprises, essentially consists of, or consists of SEQ ID NO:70 or a modified version thereof comprising modified amino acids or chemical groups. Acceptable modifications are any described herein (such as in SEQ ID NO: 89). In some embodiments, the Aβ peptide immunogen construct comprises, essentially consists of, or consists of SEQ ID NO:70. In some embodiments, the Aβ peptide immunogen construct comprises, essentially consists of, or consists of SEQ ID NO:71. In some embodiments, the Aβ peptide immunogen construct comprises, essentially consists of, or consists of SEQ ID NO:89.

In some embodiments, the C-terminal end of a peptide immunogen construct described herein is amidated.

In some embodiments, a combination of one or more (e.g., 1, 2, or 3) Tau peptide immunogen constructs and one or more (e.g., 1, 2, or 3) amyloid beta peptide immunogen constructs is used in compositions, kits and methods described herein. In some embodiments, a combination of one Tau peptide immunogen construct and one amyloid beta peptide immunogen construct is used in compositions, kits and methods described herein. In some embodiments, a composition or a kit comprises at least one Tau peptide immunogen construct, e.g., comprising or consisting of SEQ ID NO: 10, and/or comprising or consisting of SEQ ID NO: 15 (or a modified version thereof comprising modified amino acids or chemical groups, e.g., any of SEQ ID Nos: 77-81), and at least one amyloid beta peptide immunogen construct, e.g., having amino acid sequence comprising or consisting of SEQ ID NO: 70 (or a modified version thereof comprising modified amino acids or chemical groups, e.g., SEQ ID NO:89). Acceptable modifications are any described herein (such as in SEQ ID NOs:77-81 and 89). In some embodiments, a composition or a kit comprises at least one Tau peptide immunogen construct of SEQ ID NO: 10 and/or SEQ ID NO: 15, and at least one amyloid beta peptide immunogen construct of SEQ ID NO: 70.

In any of the constructs and combinations described herein, the heterologous spacer and the heterologous Th epitope can be substituted by another heterologous spacer and/or heterologous Th epitope described herein. For example, and without limitation, the heterologous spacer can be any of SEQ ID Nos: 62 and 95. Similarly, for example, and without limitation, the heterologous Th epitope can be any of SEQ ID Nos: 42, 44, 45 and 97.

The various components of the disclosed peptide immunogen constructs are described below.

a. Tau and Aβ Fragments

In some embodiments, the disclosed peptide immunogen constructs contain a B cell epitope of about 6 or more amino acids. In some embodiments, the disclosed peptide immunogen constructs contain a B cell epitope of about 8 or more amino acids. In some embodiments, the Tau or amyloid beta B cell epitope provided herein is about 6 to 40 amino acids. In some embodiments, the Tau or amyloid beta B cell epitope provided herein is about 8-40, 9-30, 10-25, 8-20, 9-18, 8-15, 10-15, 8-14, or 10-14 amino acids in length, or about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids, or any number of amino acids in between these values.

In some embodiments, the B cell epitopes described herein are modified as described herein, e.g., by acetylation of amino acids, N-methylation of amino acids, substitution of one or two amino acids by cysteine for purposes of disulfide bond formation, and/or use of N-terminal pyroglutamic acid. Acceptable modifications are any described herein (see, e.g., Table 1 and Table 3).

The Tau peptide immunogen constructs contain a B cell epitope from portions of the longest isoform of the human Tau protein (GenBank: AGF19246.1) having the amino acid sequence of SEQ ID NO: 60 shown in Table 6. The B cell epitope can be linked to a heterologous T helper cell (Th) epitope derived from pathogen proteins through an optional heterologous spacer. The disclosed peptide immunogen constructs stimulate the generation of highly specific antibodies directed against Tau. The disclosed peptide immunogen constructs can be used as an immunotherapy for patients suffering from Alzheimer's disease and/or tauopathies.

The B cell epitope portion of the Tau peptide immunogen constructs have amino acid sequences from the full-length Tau protein (SEQ ID NO: 60).

In some embodiments, the Tau B cell epitope has a sequence shown in Table 1 (any one of SEQ ID NOs: 1-8) or in Tables 7-10 (e.g., any one of SEQ ID NOs: 17-30 or 63-67).

In some embodiments, the Tau peptide (or Tau B cell epitope) of the Tau peptide immunogen constructs comprises or consists of a peptide of any one of Tables 1 and 7-10.

In some embodiments, the Tau B cell epitope comprises, consists essentially of, or consists of SEQ ID NO: 1. In some embodiments, the Tau B cell epitope comprises, consists essentially of, or consists of SEQ ID NO: 2. In some embodiments, the Tau B cell epitope comprises, consists essentially of, or consists of SEQ ID NO:3. In some embodiments, the Tau B cell epitope comprises, consists essentially of, or consists of SEQ ID NO: 4. In some embodiments, the Tau B cell epitope comprises, consists essentially of, or consists of SEQ ID NO: 5. In some embodiments, the Tau B cell epitope comprises, consists essentially of, or consists of SEQ ID NO: 6. In some embodiments, the Tau B cell epitope comprises, consists essentially of, or consists of SEQ ID NO: 7. In some embodiments, the Tau B cell epitope comprises, consists essentially of, or consists of SEQ ID NO: 8. In some embodiments, the Tau B cell epitope comprises, consists essentially of, or consists of SEQ ID NO: 202. In some embodiments, the Tau B cell epitope comprises, consists essentially of, or consists of SEQ ID NO: 17. In some embodiments, the Tau B cell epitope comprises, consists essentially of, or consists of SEQ ID NO: 18. In some embodiments, the Tau B cell epitope comprises, consists essentially of, or consists of SEQ ID NO: 19. In some embodiments, the Tau B cell epitope comprises, consists essentially of, or consists of SEQ ID NO: 20. In some embodiments, the Tau B cell epitope comprises, consists essentially of, or consists of SEQ ID NO: 21. In some embodiments, the Tau B cell epitope comprises, consists essentially of, or consists of SEQ ID NO: 22. In some embodiments, the Tau B cell epitope comprises, consists essentially of, or consists of SEQ ID NO:23. In some embodiments, the Tau B cell epitope comprises, consists essentially of, or consists of SEQ ID NO: 24. In some embodiments, the Tau B cell epitope comprises, consists essentially of, or consists of SEQ ID NO: 25. In some embodiments, the Tau B cell epitope comprises, consists essentially of, or consists of SEQ ID NO: 26. In some embodiments, the Tau B cell epitope comprises, consists essentially of, or consists of SEQ ID NO: 27. In some embodiments, the Tau B cell epitope comprises, consists essentially of, or consists of SEQ ID NO: 28. In some embodiments, the Tau B cell epitope comprises, consists essentially of, or consists of SEQ ID NO: 29. In some embodiments, the Tau B cell epitope comprises, consists essentially of, or consists of SEQ ID NO: 30. In some embodiments, the Tau B cell epitope comprises, consists essentially of, or consists of SEQ ID NO: 63. In some embodiments, the Tau B cell epitope comprises, consists essentially of, or consists of SEQ ID NO: 64. In some embodiments, the Tau B cell epitope comprises, consists essentially of, or consists of SEQ ID NO: 65. In some embodiments, the Tau B cell epitope comprises, consists essentially of, or consists of SEQ ID NO: 66. In some embodiments, the Tau B cell epitope comprises, consists essentially of, or consists of SEQ ID NO: 67.

In some embodiments, the Tau peptide (B cell epitope) of the Tau peptide immunogen constructs comprises or consists of a peptide of any one of Tables 1 and 7-10, wherein the peptide includes 1, 2, 3, 4, or 5 additional amino acids of the Tau protein of SEQ ID NO: 60 on the N-terminal end of the peptide of Table 1 or 7-10. In some embodiments, the Tau peptide (B cell epitope) of the Tau peptide immunogen constructs comprises or consists of a peptide of any one of Tables 1 and 7-10, wherein the peptide includes 1, 2, 3, 4, or 5 additional amino acids of the Tau protein of SEQ ID NO: 60 on the C-terminal end of the peptide of Table 1 or 7-10. In some embodiments, the Tau peptide (B cell epitope) of the Tau peptide immunogen constructs comprises or consists of a peptide of any one of Tables 1 and 7-10, wherein the peptide includes 1, 2, 3, 4, or 5 additional amino acids of the Tau protein of SEQ ID NO: 60 on the N-terminal end and/or the C-terminal end of the peptide of Table 1 or 7-10. The additional amino acids in the latter peptides are to be understood to be those of the Tau protein of SEQ ID NO: 60 that are contiguous with the peptide sequences of Tables 1 and 7-10.

In some embodiments, the Tau B cell epitope has a sequence of any one of SEQ ID NOs: 72-76. In some embodiments, the Tau B cell epitope comprises, consists essentially of, or consists of SEQ ID NO: 72. In some embodiments, the Tau B cell epitope comprises, consists essentially of, or consists of SEQ ID NO: 73. In some embodiments, the Tau B cell epitope comprises, consists essentially of, or consists of SEQ ID NO: 74. In some embodiments, the Tau B cell epitope comprises, consists essentially of, or consists of SEQ ID NO: 75. In some embodiments, the Tau B cell epitope comprises, consists essentially of, or consists of SEQ ID NO: 76. In some embodiments, the Tau B cell epitope comprises, consists essentially of, or consists of SEQ ID NO: 77. In some embodiments, the Tau B cell epitope comprises, consists essentially of, or consists of SEQ ID NO: 96.

In some embodiments, the Tau B cell epitope of the Tau peptide immunogen constructs comprises or consists of a peptide of any one of SEQ ID Nos: 72-76 or 96, wherein the B cell epitope includes 1, 2, 3, 4, or 5 additional amino acids of the Tau protein of SEQ ID NO: 60 on the N-terminal end of the peptide. In some embodiments, the Tau B cell epitope of the Tau peptide immunogen constructs comprises or consists of a peptide of any one of SEQ ID Nos: 72-76 or 96, wherein the B cell epitope includes 1, 2, 3, 4, or 5 additional amino acids of the Tau protein of SEQ ID NO: 60 on the C-terminal end of the peptide. In some embodiments, the Tau B cell epitope of the Tau peptide immunogen constructs comprises or consists of a peptide of any one of SEQ ID Nos: 72-76 or 96, wherein the B cell epitope includes 1, 2, 3, 4, or 5 additional amino acids of the Tau protein of SEQ ID NO: 60 on the N-terminal end and/or the C-terminal end of the peptide. The additional amino acids are to be understood to be those of the Tau protein of SEQ ID NO: 60 that are contiguous with the peptide sequences of any one of SEQ ID Nos: 72-76 and 96, respectively.

In some embodiments, the Tau peptide (B cell epitope) of the Tau peptide immunogen constructs comprises or consists of about 8 to about 40 amino acid residues from SEQ ID NO: 60 (e.g., about 8-40, 9-30, 10-25, 8-20, 9-18, or 10-15; or about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids). In some embodiments, the Tau peptide of the peptide immunogen constructs are of any one of Tables 1 and 7-10, e.g., any one of SEQ ID NOs: 1-8, 17-30, or 63-67, wherein optionally the peptide has one or more of the additional amino acids noted above. In some embodiments, the Tau peptide (B cell epitope) of the Tau peptide immunogen constructs is of any one of SEQ ID NOs: 72-76, wherein optionally the peptide has one or more of the additional amino acids noted above.

The B cell epitope portion of the amyloid beta peptide immunogen constructs have amino acid sequences from the full-length amyloid beta protein (SEQ ID NO: 68).

In some embodiments, the amyloid beta peptide (or amyloid beta B cell epitope) of the amyloid beta peptide immunogen constructs comprises or consists of a peptide of any one of Table 3.

In some embodiments, the amyloid beta B cell epitope comprises, consists essentially of, or consists of SEQ ID NO: 69. In some embodiments, the amyloid beta B cell epitope comprises, consists essentially of, or consists of SEQ ID NO: 82. In some embodiments, the amyloid beta B cell epitope comprises, consists essentially of, or consists of SEQ ID NO:83. In some embodiments, the amyloid beta B cell epitope comprises, consists essentially of, or consists of SEQ ID NO: 84. In some embodiments, the amyloid beta B cell epitope comprises, consists essentially of, or consists of SEQ ID NO: 85. In some embodiments, the amyloid beta B cell epitope comprises, consists essentially of, or consists of SEQ ID NO: 86. In some embodiments, the amyloid beta B cell epitope comprises, consists essentially of, or consists of SEQ ID NO: 87. In some embodiments, the amyloid beta B cell epitope comprises, consists essentially of, or consists of SEQ ID NO: 88. In some embodiments, the amyloid beta B cell epitope comprises, consists essentially of, or consists of any of the sequences of amyloid beta peptides or B cell epitopes described in WO 2014/143087 or U.S. Pat. No. 9,102,752 B2.

In some embodiments, the B cell epitope of the amyloid beta peptide immunogen constructs comprises or consists of a peptide of any one of SEQ ID Nos: 69 and 82-88, wherein the B cell epitope includes 1, 2, 3, 4, or 5 additional amino acids of the amyloid beta protein of SEQ ID NO: 68 on the N-terminal end of the peptide. In some embodiments, the B cell epitope of the amyloid beta peptide immunogen constructs comprises or consists of a peptide of any one of SEQ ID Nos: 69 and 82-88, wherein the B cell epitope includes 1, 2, 3, 4, or 5 additional amino acids of the amyloid beta protein of SEQ ID NO: 68 on the C-terminal end of the peptide. In some embodiments, the B cell epitope of the amyloid beta peptide immunogen constructs comprises or consists of a peptide of any one of SEQ ID Nos: 69 and 82-88, wherein the B cell epitope includes 1, 2, 3, 4, or 5 additional amino acids of the amyloid beta protein of SEQ ID NO: 68 on the N-terminal end and/or the C-terminal end of the peptide. The additional amino acids are to be understood to be those of the amyloid protein of SEQ ID NO: 68 that are contiguous with the peptide sequences of any one of SEQ ID Nos: 69 and 82-88, respectively.

As noted above, in some embodiments, the Aβ peptide immunogen construct is selected from SEQ ID NO: 70 and SEQ ID NO: 71 or a combination thereof is used (see Table 4). Also see WO 2014/143087 for additional Aβ peptides and Aβ peptide immunogenic constructs that can be used in combination with Tau peptide immunogen constructs described herein. Peptides immunogen constructs falling within the formulae of WO 2014/143087 can also be used and are included in this disclosure.

In some embodiments, the C-terminus of a peptide immunogenic construct described herein is amidated.

b. Heterologous T Helper Cell Epitopes (Th Epitopes)

The present disclosure provides peptide immunogen constructs containing a B cell epitope from Tau or Aβ covalently linked to a heterologous T helper cell (Th) epitope directly or through an optional heterologous spacer.

The heterologous Th epitope in the peptide immunogen constructs enhance the immunogenicity of the Tau or Aβ fragments, which facilitates the production of specific high titer antibodies directed against the optimized target B cell epitope (e.g., the Tau fragment) through rational design.

The term “heterologous”, as used herein, refers to an amino acid sequence that is derived from an amino acid sequence that is not part of, or homologous with, the wild-type sequence of Tau or Aβ. Thus, in the case of Tau, a heterologous Th epitope is a Th epitope derived from an amino acid sequence that is not naturally found in Tau (i.e., the Th epitope is not autologous to Tau). Since the Th epitope is heterologous to Tau, the natural amino acid sequence of Tau is not extended in either the N-terminal or C-terminal directions when the heterologous Th epitope is covalently linked to the Tau fragment. Similarly, in the case of Aβ, a heterologous Th epitope is a Th epitope derived from an amino acid sequence that is not naturally found in Aβ (i.e., the Th epitope is not autologous to Aβ). Since the Th epitope is heterologous to Aβ, the natural amino acid sequence of Aβ is not extended in either the N-terminal or C-terminal directions when the heterologous Th epitope is covalently linked to the Aβ fragment.

The heterologous Th epitope of the present disclosure can be any Th epitope that does not have an amino acid sequence naturally found in Tau (or Aβ). The Th epitope can have an amino acid sequence derived from any species (e.g., human, pig, cattle, dog, rat, mouse, guinea pigs, etc.). The Th epitope can also have promiscuous binding motifs to MHC class II molecules of multiple species. In certain embodiments, the Th epitope comprises multiple promiscuous MHC class II binding motifs to allow maximal activation of T helper cells leading to initiation and regulation of immune responses. The Th epitope is preferably immunosilent on its own, i.e., little, if any, of the antibodies generated by the peptide immunogen constructs will be directed towards the Th epitope, thus allowing a very focused immune response directed to the targeted B cell epitope of the Tau or Aβ fragment.

Th epitopes of the present disclosure include, but are not limited to, amino acid sequences derived from foreign pathogens, as exemplified in Table 5 (any one of SEQ ID NOs: 31 to 59, or SEQ ID NO: 97). Further, Th epitopes include idealized artificial Th epitopes and combinatorial idealized artificial Th epitopes (e.g., any one of SEQ ID NOs: 32 and 39-45). The heterologous Th epitope peptides presented as a combinatorial sequence (e.g., any one of SEQ ID NOs: 40-43), contain a mixture of amino acid residues represented at specific positions within the peptide framework based on the variable residues of homologues for that particular peptide. An assembly of combinatorial peptides can be synthesized in one process by adding a mixture of the designated protected amino acids, instead of one particular amino acid, at a specified position during the synthesis process. Such combinatorial heterologous Th epitope peptides assemblies can allow broad Th epitope coverage for animals having a diverse genetic background. Representative combinatorial sequences of heterologous Th epitope peptides include SEQ ID NOs: 40-43 which are shown in Table 5. Th epitope peptides of the present invention provide broad reactivity and immunogenicity to animals and patients from genetically diverse populations.

In some embodiments, in the Th epitopes shown in Table 5 that comprise N-terminal lysines (such as the “KKK” amino acid sequence at the N-terminal end), the N-terminal lysines can be removed for use in the peptide immunogen constructs described herein (e.g., for use in the peptide immunogen constructs comprising a spacer comprising one or more lysines). In some embodiments, in the Th epitopes shown in Table 5 that comprise the “KKK” amino acid sequence at the N-terminal end, the “KKK” amino acid sequence can be removed for use in the peptide immunogen constructs described herein (e.g., for use in the peptide immunogen constructs comprising a spacer comprising “KKK” amino acid sequence). See, for example, the peptide immunogen construct of SEQ ID NO:71, wherein the sequence of the UBITh®2 Th epitope in the peptide immunogen construct lacks three N-terminal lysines.

In some embodiments, the heterologous Th epitope comprises or has the amino acid sequence of SEQ ID NO: 44. In some embodiments, the heterologous Th epitope comprises or has the amino acid sequence of SEQ ID NO: 42. In some embodiments, the heterologous Th epitope comprises or has the amino acid sequence of SEQ ID NO: 45. In some embodiments, the heterologous Th epitope comprises or has the amino acid sequence of SEQ ID NO: 97.

Peptide immunogen constructs comprising Th epitopes are or can be produced simultaneously in a single solid-phase peptide synthesis in tandem with the B cell epitope-containing fragment. Th epitopes also include immunological analogues of Th epitopes. Immunological Th analogues include immune-enhancing analogs, cross-reactive analogues and segments of any one of these Th epitopes that are sufficient to enhance or stimulate an immune response to the B cell epitope-containing fragments.

Functional immunologically analogues of the Th epitope peptides are also effective and included as part of the present invention. Functional immunological Th analogues can include conservative substitutions, additions, deletions, and insertions of from one to about five amino acid residues in the Th epitope which do not essentially modify the Th-stimulating function of the Th epitope. The conservative substitutions, additions, and insertions can be accomplished with natural or non-natural amino acids. Table 5 identifies another variation of a functional analogue for Th epitope peptide. In particular, SEQ ID NOs: 32 and 39 of MvF1 and MvF2 Th are functional analogues of SEQ ID NOs: 42 and 44 of MvF4 and MvF5 in that they differ in the amino acid frame by the deletion (SEQ ID NOs: 32 and 39) or the inclusion (SEQ ID NOs: 42 and 44) of two amino acids each at the N- and C-termini. The differences between these two series of analogous sequences would not affect the function of the Th epitopes contained within these sequences. Therefore, functional immunological Th analogues include several versions of the Th epitope derived from Measles Virus Fusion protein MvF1-4 Ths (e.g., any one of SEQ ID NOs: 32, 39, 40, 42, and 44) and from Hepatitis Surface protein HBsAg 1-3 Ths (e.g., any one of SEQ ID NOs: 41, 43, and 45). In some embodiments, a sequence of SEQ ID NO: 44 is used. In some embodiments, the sequence of SEQ ID NO: 42 is used.

The Th epitope in the peptide immunogen constructs can be covalently linked at either N- or C-terminal end of the B cell epitope-containing peptides. In some embodiments, the Th epitope is covalently linked to the N-terminal end of the B cell epitope-containing peptide. In other embodiments, the Th epitope is covalently linked to the C-terminal end of the B cell epitope-containing peptide. In certain embodiments, more than one Th epitope is covalently linked to the B cell epitope-containing fragment. When more than one Th epitope is linked to the B cell epitope-containing fragment, each Th epitope can have the same amino acid sequence or different amino acid sequences. In addition, when more than one Th epitope is linked to the B cell epitope-containing fragment, the Th epitopes can be arranged in any order. For example, the Th epitopes can be consecutively linked to the N-terminal end of the B cell epitope-containing fragment, or consecutively linked to the C-terminal end of the B cell epitope-containing fragment, or a Th epitope can be covalently linked to the N-terminal end of the B cell epitope-containing fragment while a separate Th epitope is covalently linked to the C-terminal end of the B cell epitope-containing fragment. There is no limitation in the arrangement of the Th epitopes in relation to the B cell epitope-containing fragment. For the immunogen constructs depicted in Tables 2 and 4, it should be understood that the disclosure includes corresponding immunogenic constructs in which the positions of the B cell epitope-containing peptide and the Th epitope are reversed from the orientation set forth in Tables 2 and 4. In some embodiments, for the immunogen constructs depicted in Tables 2 and 4, it should also be understood that another Th epitope can be used (i.e., the provided Th epitope can be replaced by another described herein or known in the art).

In some embodiments, the Th epitope is covalently linked to the B cell epitope-containing fragment directly. In other embodiments, the Th epitope is covalently linked to the B cell epitope-containing fragment through a heterologous spacer described in further detail below.

c. Heterologous Spacer

The disclosed B cell epitope-containing peptide immunogen constructs optionally contain a heterologous spacer that covalently links the B cell epitope from B cell epitope-containing to the heterologous T helper cell (Th) epitope.

As discussed above, the term “heterologous,” refers to an amino acid sequence that is derived from an amino acid sequence that is not part of, or homologous with, the wild-type sequence of Tau or Aβ. Thus, the natural amino acid sequence of Tau or Aβ is not extended in either the N-terminal or C-terminal directions when the heterologous spacer is covalently linked to the B cell epitope from Tau or Aβ because the spacer is heterologous to the Tau or Aβ sequence.

The spacer is any molecule or chemical structure capable of linking two amino acids and/or peptides together. The spacer can vary in length or polarity depending on the application. The spacer attachment can be through an amide- or carboxyl-linkage but other functionalities are possible as well. The spacer can include a chemical compound, a naturally occurring amino acid, or a non-naturally occurring amino acid.

The spacer can provide structural features to the Tau or beta amyloid peptide immunogen construct. Structurally, the spacer provides a physical separation of the Th epitope from the B cell epitope of the Tau or beta amyloid fragment. The physical separation by the spacer can disrupt any artificial secondary structures created by joining the Th epitope to the B cell epitope. Additionally, the physical separation of the epitopes by the spacer can eliminate interference between the Th cell and/or B cell responses. Furthermore, the spacer can be designed to create or modify a secondary structure of the peptide immunogen construct. For example, a spacer can be designed to act as a flexible hinge to enhance the separation of the Th epitope and B cell epitope. A flexible hinge spacer can also permit more efficient interactions between the presented peptide immunogen and the appropriate Th cells and B cells to enhance the immune responses to the Th epitope and B cell epitope. Examples of sequences encoding flexible hinges are found in the immunoglobulin heavy chain hinge region, which are often proline rich. One particularly useful flexible hinge that can be used as a spacer is provided by the sequence Pro-Pro-Xaa-Pro-Xaa-Pro (SEQ ID NO: 61), where Xaa is any amino acid, and preferably aspartic acid.

The spacer can also provide functional features to the peptide immunogen constructs. For example, the spacer can be designed to change the overall charge of the peptide immunogen constructs, which can affect the solubility of the peptide immunogen construct. Additionally, changing the overall charge of the peptide immunogen constructs can affect the ability of the peptide immunogen construct to associate with other compounds and reagents. As discussed in further detail below, the peptide immunogen constructs can be formed into a stable immunostimulatory complex with a highly charged oligonucleotide, such as CpG oligomers through electrostatic association. The overall charge of the peptide immunogen constructs is important for the formation of these stable immunostimulatory complexes.

In some embodiments, the heterologous spacer comprises or consists of one of the following amino acids: Lys-, Gly-, Lys-Lys-Lys-, (α, ε-N)Lys, Lys-Lys-Lys-εLys, ε-N-Lys-Lys-Lys-Lys, and Pro-Pro-Xaa-Pro-Xaa-Pro, wherein Xaa is any amino acid or is aspartic acid. In some embodiments, the spacer comprises or consists of the amino acid sequence Lys-Lys-Lys, ε-Lys-Lys-Lys-Lys or Lys-Lys-Lys-ε-Lys. In some embodiments, the sequence of the spacer comprises or consists of ε-Lys-Lys-Lys-Lys or Lys-Lys-Lys-ε-Lys. In some embodiments, the heterologous spacer is Lys-Lys-Lys-εLys. In some embodiments, the heterologous spacer is εLys-Lys-Lys-Lys.

As is understood in the art, for a construct including ε-Lys as or within a spacer, the C-terminus of a first peptide sequence is conjugated to the epsilon amino group of the lysine to form a first amide bond, and the N-terminus of a second peptide sequence is conjugated to the carboxyl group of the lysine to form a second amide bond.

The structure of the Lys-ε-Lys (K-εK) linkage is shown below:

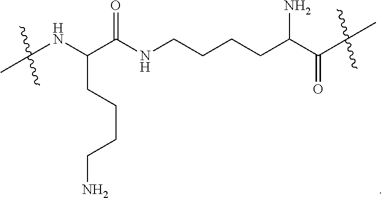 [SMILES: *NC(CCCCN)C(=O)NCCCCC(N)C(*)=O]

Chemical compounds that can be used as a spacer include, but are not limited to, (2-aminoethoxy) acetic acid (AEA), 5-aminovaleric acid (AVA), 6-aminocaproic acid (Ahx), 8-amino-3,6-dioxaoctanoic acid (AEEA, mini-PEG1), 12-amino-4,7,10-trioxadodecanoic acid (mini-PEG2), 15-amino-4,7,10,13-tetraoxapenta-decanoic acid (mini-PEG3), trioxatridecan-succinamic acid (Ttds), 12-amino-dodecanoic acid, Fmoc-5-amino-3-oxapentanoic acid (O1Pen), and the like.

Naturally occurring amino acids include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine.

Non-naturally occurring amino acids include, but are not limited to, ε-N Lysine, β-alanine, ornithine, norleucine, norvaline, hydroxyproline, thyroxine, γ-amino butyric acid, homoserine, citrulline, aminobenzoic acid, 6-aminocaproic acid (Aca; 6-Aminohexanoic acid), hydroxyproline, mercaptopropionic acid (MPA), 3-nitro-tyrosine, pyroglutamic acid, and the like.

The spacer in the peptide immunogen constructs can be covalently linked at either N- or C-terminal end of the Th epitope and the B cell epitope-containing peptide. In some embodiments, the spacer is covalently linked to the C-terminal end of the Th epitope and to the N-terminal end of the B cell epitope-containing peptide. In other embodiments, the spacer is covalently linked to the C-terminal end of the B cell epitope-containing peptide and to the N-terminal end of the Th epitope. In certain embodiments, more than one spacer can be used, for example, when more than one Th epitope is present in the peptide immunogen construct. When more than one spacer is used, each spacer can be the same as each other or different. Additionally, when more than one Th epitope is present in the peptide immunogen construct, the Th epitopes can be separated with a spacer, which can be the same as, or different from, the spacer used to separate the Th epitope from the B cell epitope. There is no limitation in the arrangement of the spacer in relation to the Th epitope or the B cell epitope-containing fragment.

In certain embodiments, the heterologous spacer is a naturally occurring amino acid or a non-naturally occurring amino acid. In other embodiments, the spacer contains more than one naturally occurring or non-naturally occurring amino acids. In some embodiments, the spacer comprises at least 3, or at least 4 amino acids (e.g., lysines). In some embodiments, the spacer comprises less than 7, 6, or 5 amino acids. In specific embodiments, the spacer is Lys-, Gly-, Lys-Lys-Lys-, (α, ε-N)Lys, or ε-N-Lys-Lys-Lys-Lys (SEQ ID NO: 62).

d. Modified B Cell Epitopes and Peptide Immunogen Constructs

In some embodiments, provided herein are modified Tau and Aβ B cell epitopes. The specific modified B cell epitopes provided herein are shown in Table 1 and Table 3.

In some embodiments, provided herein are modified Tau and Aβ peptide immunogen constructs. The specific modified peptide immunogen constructs provided herein are shown in Table 2 and Table 4.

In some embodiments, a Tau B cell epitope comprises an N-methylated amino acid or amino acids. N-methylated amino acids are mono methylated and attached to the following amino acid by an amide bond between the N-methylated amine of an amino acid and the carboxylic acid of the following amino acid. One, two or more amino acids can be N-methylated. In some embodiments, N-methylated amino acids are isoleucine and/or lysine. In some embodiments, one isoleucine in the B cell epitope is N-methylated. In some embodiments, one lysine in the B cell epitope is N-methylated. In some embodiments, one isoleucine and one lysine in the B cell epitope are N-methylated. In some embodiments, N-methylated amino acids appear at the N-terminal end of a B cell epitope (such as a Tau B cell epitope). N-methylated amino acids are present in Tau B cell epitopes of SEQ ID Nos: 73-75. Examples of N-methylated amino acid analogs are shown below:

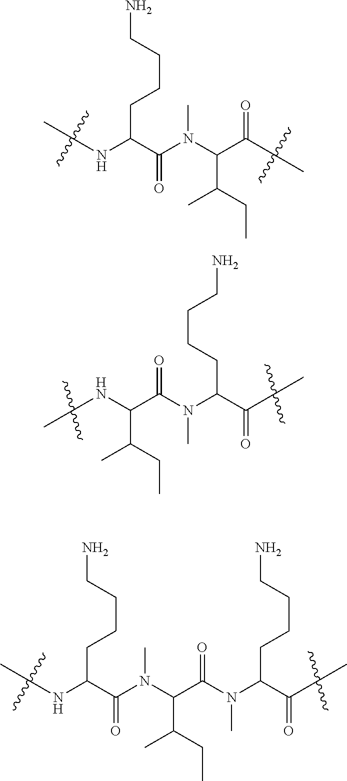

In some embodiments, a Tau B cell epitope comprises an acetylated amino acid or amino acids. In some embodiments, isoleucine (e.g., one isoleucine of the B cell epitope) is acetylated. In some embodiments, acetylated amino acid is at the N-terminal end of a B cell epitope (such as a Tau B cell epitope). An acetylated amino acid is present in Tau B cell epitope of SEQ ID No: 72. In some embodiments, a Th epitope comprises an acetylated amino acid or amino acids. In some embodiments, acetylated amino acid is at the N-terminal end of a Th epitope. In some embodiments, isoleucine (e.g., one isoleucine of the Th epitope) is acetylated. Examples of acetylated amino acids contemplated herein are shown below (in particular, an N-acetylated isoleucine):

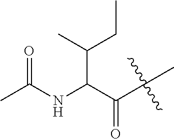 [SMILES: *C(=O)C(NC(C)=O)C(C)CC]

In some embodiments, a Tau B cell epitope, a Th epitope or a peptide immunogen construct, instead of an acetylated amino acid, comprises another modification (as an alternative to acetylation) protecting the N-terminal end from protease degradation. In some embodiments, such another modification is any modification known in the art (that can protect the N-terminal end from protease degradation). In some embodiments, a Tau B cell epitope, a Th epitope or a peptide immunogen construct, comprises one of the following modifications at the N-terminal end: one or more additional glycines (e.g., three additional glycines or GGG), propionylation, a benzyloxycarbonyl group, a short PEG, or a D-amino acid. In some embodiments, a Tau B cell epitope, a Th epitope or a peptide immunogen construct, comprises three glycines (which are not part of the B cell epitope) at the N-terminal end. In some embodiments, a Tau B cell epitope, a Th epitope or a peptide immunogen construct, is propanoylated at the N-terminal end. In some embodiments, a Tau B cell epitope, a Th epitope or a peptide immunogen construct, comprises benzyloxycarbonyl at the N-terminal end. In some embodiments, a Tau B cell epitope, a Th epitope or a peptide immunogen construct, comprises a PEG (e.g., a short PEG) at the N-terminal end. In some embodiments, a Tau B cell epitope, a Th epitope or a peptide immunogen construct, comprises a D-amino acid at the N-terminal end. In other embodiments, a Tau B cell epitope, a Th epitope or a peptide immunogen construct, is acetylated at the N-terminal end.

In some embodiments, a Tau B cell epitope comprises one or two amino acid substitutions wherein a non-cysteine amino acid is replaced with a cysteine, such that the resulting B cell epitope comprises two cysteines that can form a disulfide bond (resulting in cyclization by cysteine disulfide bonds). Such substituted amino acids are present in Tau B cell epitope of SEQ ID No: 76.

In some embodiments, an amyloid beta B cell epitope comprises a pyroglutamic acid. In some embodiments, an amyloid beta B cell epitope comprises a pyroglutamic acid at the N-terminal end of a B cell epitope. Such modification is present in amyloid beta B cell epitope of SEQ ID No: 82. Pyroglutamic acid is a derivative of Glutamic acid in which the side chain underwent dehydration resulting in an amide bond between the side chain acid group and the alpha amino group, as shown below:

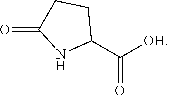

The specific B cell epitope modifications described herein (such as in SEQ ID Nos: 72-76 and 82) are based upon predicted structures of Tau and amyloid beta peptide sequences and/or empirically determined tryptic digest products.

In some embodiments, the modifications described herein improve protease stability of the peptides or impart protease stability to the peptides.

e. Specific Embodiments of the Tau and Aβ Peptide Immunogen Constructs and Combinations

The Tau peptide immunogen construct can be represented by the formulae:

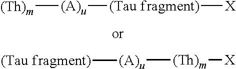

wherein
Th is a heterologous T helper epitope;
A is a heterologous spacer;
(Tau fragment) is a B cell epitope having 6 to about 40, or about 8 to about 40, amino acid residues from the full-length Tau protein of SEQ ID NO: 60 (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids, or any length range in between these values);
X is an α-COOH or α-CONH₂ of an amino acid;
m is from 1 to about 4; and
n is from 0 to about 10.

In certain embodiments, the heterologous Th epitope in the Tau peptide immunogen construct has an amino acid sequence selected from any one of those set forth in Table 5. In certain embodiments, the Tau peptide immunogen construct contains more than one Th epitope. In certain embodiments, the Tau peptide immunogen construct contains more than one Th epitope, e.g., of SEQ ID NO:44.

In certain embodiments, the optional heterologous spacer is selected from any one of Lys-, Gly-, Lys-Lys-Lys-, (α, ε-N)Lys, ε-N-Lys-Lys-Lys-Lys (SEQ ID NO: 62), or Lys-Lys-Lys-ε-Lys (SEQ ID NO:95), and combinations thereof. In specific embodiments, the heterologous spacer is ε-N-Lys-Lys-Lys-Lys (SEQ ID NO: 62). In specific embodiments, the heterologous spacer is Lys-Lys-Lys-ε-Lys (SEQ ID NO:95).

In certain embodiments, the Tau fragment has about 8 to about 40 (e.g., 10-30, 10-25, 12-20, or 12-15) amino acid residues from the full-length Tau protein of SEQ ID NO: 60. In some embodiments, the Tau fragment has about 8-20, 8-15, 10-15, or 10-14 amino acid residues from the full-length Tau protein of SEQ ID NO: 60. In specific embodiments, the Tau fragment has an amino acid sequence shown in Table 1 (e.g., one of SEQ ID NOs: 1-8) or in one of Tables 7-10 (e.g., one of SEQ ID NOs: 17-30 or 63-67). In other specific embodiments, the Tau fragment has an amino acid sequence of any one of SEQ ID NOs: 72-76 shown in Table 1.

In some embodiments, the tau peptide immunogen construct is selected from:

a.
SEQ ID NO: 9
MAEPRQEFEVMEDHAGTYGLGDKKK-eK-
ISITEIKGVIVHRIETILF-NH2;
b.
SEQ ID NO: 10
DHAGTYGLGDKKK-eK-ISITEIKGVIVHRIETILF-NH2;
c.
SEQ ID NO: 11
ISITEIKGVIVHRIETILF-eK-KKKNITHVPGGGNKK-NH2;
d.
SEQ ID NO: 12
ISITEIKGVIVHRIETILF-eK-KKKKDNIKHVPGGGSVQIVYK-
NH2;
e.
SEQ ID NO: 13
ISITEIKGVIVHRIETILF-eK-KKKTKIATPRGAAPP-NH2;
f.
SEQ ID NO: 14
ISITEIKGVIVHRIETILF-cK-KKKVVRTPPKSPSSAKSRL-NH2;
g.
SEQ ID NO: 15
ISITEIKGVIVHRIETILF-eK-KKKIKHVPGGGSVQIVYK-NH2;
and
h.
SEQ ID NO: 16
ISITEIKGVIVHRIETILF-eK-KKKVSGDTSPRHLSNVSST-NH2

In some embodiments, the T cell epitope of any one of SEQ ID NOs: 9-16 is replaced with a T cell epitope of any one of SEQ ID NOs: 31-59. In some embodiments, the positions of the B cell epitope and the T cell epitope of any one of SEQ ID NOs: 9-16 is reversed within the molecule (i.e., if the B cell epitope is at the N-terminal end of the original molecule, it is placed at the C-terminal end of the molecule in the reversed molecule, etc.).

In some embodiments, the Tau peptide immunogen construct is selected from any one of SEQ ID Nos:77-81. In some embodiments, the T cell epitope of any one of SEQ ID NOs: 77-81 is replaced with a T cell epitope of any one of SEQ ID NOs: 31-59. In some embodiments, the positions of the B cell epitope and the T cell epitope of any one of SEQ ID Nos:77-81 is reversed within the molecule (i.e., if the B cell epitope is at the N-terminal end of the original molecule, it is placed at the C-terminal end of the molecule in the reversed molecule, etc.).

The Aβ peptide immunogen construct can be represented by the formulae:

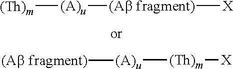

wherein
Th is a heterologous T helper epitope;
A is a heterologous spacer;
(Aβ fragment) is a B cell epitope having 6 to about 40, or about 8 to about 40, amino acid residues from the full-length Aβ protein of SEQ ID NO: 68 (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids, or any length range in between these values);
X is an α-COOH or α-CONH₂ of an amino acid;
m is from 1 to about 4; and
n is from 0 to about 10.

In certain embodiments, the heterologous Th epitope in the Aβ peptide immunogen construct has an amino acid sequence selected from any one of those set forth in Table 5. In certain embodiments, the Aβ peptide immunogen construct contains more than one Th epitope. In certain embodiments, the Aβ peptide immunogen construct contains more than one Th epitope, e.g., of SEQ ID NO:44.

In certain embodiments, the optional heterologous spacer is selected from any one of Lys-, Gly-, Lys-Lys-Lys-, (α, ε-N)Lys, ε-N-Lys-Lys-Lys-Lys (SEQ ID NO: 62), or Lys-Lys-Lys-ε-Lys (SEQ ID NO:95), and combinations thereof. In specific embodiments, the heterologous spacer is ε-N-Lys-Lys-Lys-Lys (SEQ ID NO: 62). In specific embodiments, the heterologous spacer is Lys-Lys-Lys-ε-Lys (SEQ ID NO:95).

In certain embodiments, the Aβ fragment has about 8 to about 40 (e.g., 10-30, 10-25, 12-20, or 12-15) amino acid residues from the full-length Tau protein of SEQ ID NO: 60. In some embodiments, the Aβ fragment has about 8-20, 8-15, 10-15, or 10-14 amino acid residues from the full-length Aβ protein of SEQ ID NO: 68. In specific embodiments, the Aβ fragment has an amino acid sequence shown in Table 3 (e.g., one of SEQ ID NOs: 68 or 69). In other specific embodiments, the Aβ fragment has an amino acid sequence of any one of SEQ ID NOs: 83-88 shown in Table 3.

In some embodiments, the Aβ peptide immunogen construct is selected from SEQ ID NOs:70, 71 and 89.

In some embodiments, the T cell epitope of any one of SEQ ID NOs: 70, 71 and 89 is replaced with a T cell epitope of any one of SEQ ID NOs: 31-59. In some embodiments, the positions of the B cell epitope and the T cell epitope of any one of SEQ ID NOs: 70, 71 and 89 is reversed within the molecule (i.e., if the B cell epitope is at the N-terminal end of the original molecule, it is placed at the C-terminal end of the molecule in the reversed molecule, etc.).

In some embodiments, provided herein is a combination of any two, three or more Tau peptide immunogen constructs described herein.

In some embodiments, provided herein is a combination of any one, two or more Tau peptide immunogen constructs described herein, with any one or more Aβ peptide immunogen constructs described herein. In some embodiments, the combination is formulated in a single composition, as described herein. In some embodiments, such two or more peptide immunogen constructs are present in the same kit, as described herein.

f. Variants, Homologues, and Functional Analogues

Variants and analogs of the above immunogenic peptides that induce and/or cross-react with antibodies to the preferred epitopes of Tau or Aβ protein can also be used. Analogs, including allelic, species, and induced variants, typically differ from naturally occurring peptides at one, two, or a few positions, often by virtue of conservative substitutions. Analogs typically exhibit at least 80 or 90% sequence identity with natural peptides. Some analogs also include unnatural amino acids or modifications of N- or C-terminal amino acids at one, two, or a few positions.

Variants that are functional analogues can have a conservative substitution in an amino acid position; a change in overall charge: a covalent attachment to another moiety; or amino acid additions, insertions, or deletions; and/or any combination thereof.

Conservative substitutions are when one amino acid residue is substituted for another amino acid residue with similar chemical properties. For example, the nonpolar (hydrophobic) amino acids include alanine, leucine, isoleucine, valine, proline, phenylalanine, tryptophan and methionine; the polar neutral amino acids include glycine, serine, threonine, cysteine, tyrosine, asparagine, and glutamine; the positively charged (basic) amino acids include arginine, lysine and histidine; and the negatively charged (acidic) amino acids include aspartic acid and glutamic acid.

In a particular embodiment, the functional analogue has at least 50% identity to the original amino acid sequence. In another embodiment, the functional analogue has at least 80% identity to the original amino acid sequence. In yet another embodiment, the functional analogue has at least 85% identity to the original amino acid sequence. In still another embodiment, the functional analogue has at least 90% identity to the original amino acid sequence.

Variants also include variations to the phosphorylated residues. For example, variants can include different residues within the peptides that are phosphorylated. Variant immunogenic Tau or Aβ peptides can also include pseudo-phosphorylated peptides. The pseudo-phosphorylated peptides are generated by substituting one or more of the phosphorylated serine, threonine, and tyrosine residues of the Tau or Aβ peptides with acidic amino acid residues such as glutamic acid and aspartic acid.

### Tau Isoforms Targeted by the Tau Peptide Immunogen Constructs and Combinations

Tau is expressed in six isoforms, reflecting alternative splicing of the same gene. These isoforms comprise splice variants at the N terminus (0N, 1N, and 2N forms) and within the repeat domain (3R and 4R forms), yielding combinations 0N3R, N3R, 2N3R, 0N4R, 1N4R, and 2N4R (see, e.g., Wu et al., 2022, Frontiers in Aging Science 14: Article 945875, doi: 10.3389/fnagi.2022.945875, which is incorporated by reference herein in its entirety and in particular for its disclosure of Tau splice variants). Important considerations need to be taken when targeting Tau, as specific Tau isoforms are enriched differently in various tauoplathies. Ideally, an effective Tau targeting therapy would be effective across all Tau isoforms. 4R Tau targeting therapies could be effective for AD and other tauopathies with 4R enrichment. However, a therapy targeting 4R but not 3R isoform of Tau would likely be ineffective in the treatment of Pick's disease where 3R Tau is present.

In some embodiments, the Tau peptide immunogen constructs provided herein elicit antibodies capable of binding at least two, three, four, five or each of isoforms of Tau selected from: 0N3R, 1N3R, 2N3R, 0N4R, 1N4R, and 2N4R. The amino acid sequences of such Tau isoforms are set forth in Table 6 (in particular, 2N4R isoform has SEQ ID NO:60, 0N3R isoform has SEQ ID NO: 90, 1N3R isoform has SEQ ID NO: 91; 2N3R isoform has SEQ ID NO: 92, 0N4R isoform has SEQ ID NO: 93, and 1N4R isoform has SEQ ID NO: 94). In some embodiments, the Tau peptide immunogen constructs provided herein elicit antibodies capable of binding each of the isoforms of Tau of SEQ ID Nos: 60 and 90-94. In some embodiments, the Tau peptide immunogen construct provided herein comprising a B cell epitope from the N-terminal region of Tau (within amino acids 1-44) elicits antibodies binding to at least two, three, four, five or each isoforms of Tau selected from: 0N3R, 1N3R, 2N3R, 0N4R, 1N4R, and 2N4R. In some embodiments, the Tau peptide immunogen construct provided herein comprising a B cell epitope from the N-terminal region of Tau (within amino acids 1-44) elicits antibodies binding to at least two, three, four, five or each of SEQ ID Nos: 60, and 90-94. In some embodiments, the Tau peptide immunogen construct provided herein comprising a B cell epitope from the N-terminal region of Tau (within amino acids 1-44) elicits antibodies binding to each of SEQ ID Nos: 60, and 90-94. In some embodiments, the Tau peptide immunogen construct provided herein comprising a B cell epitope from the repeat domain of Tau (within amino acids 244-372, or within amino acids 250-320) elicits antibodies binding to at least two, three, four, five or each isoforms of Tau selected from: 0N3R, 1N3R, 2N3R, 0N4R, 1N4R, and 2N4R. In some embodiments, the Tau peptide immunogen construct provided herein comprising a B cell epitope from the repeat domain of Tau (within amino acids 244-372) elicits antibodies binding to at least two, three, four, five or each of SEQ ID Nos: 60, and 90-94. In some embodiments, the Tau peptide immunogen construct provided herein comprising a B cell epitope from the repeat domain of Tau (within amino acids 244-372) elicits antibodies binding to each of SEQ ID Nos: 60, and 90-94. In some embodiments, the Tau peptide immunogen construct provided herein comprising a B cell epitope from the repeat domain of Tau (within amino acids 244-372) elicits antibodies that can bind to two epitopes in Tau isoforms comprising 4R, and one epitope in Tau isoforms comprising 3R.

In some embodiments, the Tau peptide immunogen construct comprising, essentially consisting of, or consisting of SEQ ID NO: 10 elicits antibodies binding to each of SEQ ID Nos: 60, and 90-94. In some embodiments, the Tau peptide immunogen construct comprising, essentially consisting of, or consisting of SEQ ID NO: 15 elicits antibodies binding to each of SEQ ID Nos: 60, and 90-94. In some embodiments, the Tau peptide immunogen construct comprising, essentially consisting of, or consisting of SEQ ID NO: 15 elicits antibodies that can bind to at least one epitope in Tau isoforms comprising 4R. In some embodiments, the Tau peptide immunogen construct comprising, essentially consisting of, or consisting of SEQ ID NO: 15 elicits antibodies that can bind to two epitopes in Tau isoforms comprising 4R. In some embodiments, the Tau peptide immunogen construct comprising, essentially consisting of, or consisting of SEQ ID NO: 15 elicits antibodies that can bind to one epitope in Tau isoforms comprising 3R. In some embodiments, the Tau peptide immunogen construct comprising, essentially consisting of, or consisting of SEQ ID NO: 15 elicits antibodies that can bind to P301S mutant form of Tau. In some embodiments, the Tau peptide immunogen construct comprising, essentially consisting of, or consisting of SEQ ID NO: 15 elicits antibodies that can bind to P301L mutant form of Tau. In some embodiments, a composition or combination comprising Tau peptide immunogen construct comprising, essentially consisting of, or consisting of SEQ ID NO: 15 and Tau peptide immunogen construct comprising, essentially consisting of, or consisting of SEQ ID NO: 10 elicits antibodies that can bind to Tau isoforms comprising 4R. In some embodiments, a composition or combination comprising Tau peptide immunogen construct comprising, essentially consisting of, or consisting of SEQ ID NO: 15 and Tau peptide immunogen construct comprising, essentially consisting of, or consisting of SEQ ID NO: 10 peptide elicits antibodies that can bind to Tau isoforms comprising 3R. In some embodiments, a composition or combination comprising Tau peptide immunogen construct comprising, essentially consisting of, or consisting of SEQ ID NO: 15 and Tau peptide immunogen construct comprising, essentially consisting of, or consisting of SEQ ID NO: 10 elicits antibodies that can bind to P301S mutant form of Tau. In some embodiments, a composition or combination comprising Tau peptide immunogen construct comprising, essentially consisting of, or consisting of SEQ ID NO: 15 and Tau peptide immunogen construct comprising, essentially consisting of, or consisting of SEQ ID NO: 10 elicits antibodies that can bind to P301L mutant form of Tau.

### Compositions

The present disclosure also provides compositions comprising one, two or more of the disclosed Tau peptide immunogen constructs, and/or one or more of the disclosed Tau peptide immunogen constructs in combination with one or more Aβ peptide immunogen constructs.

a. Peptide Compositions

Compositions containing one, two or more disclosed Tau peptide immunogen constructs, optionally in combination with one or more Aβ peptide immunogen constructs, can be in liquid or solid form. Liquid compositions can include water, buffers, solvents, salts, and/or any other acceptable reagent that does not alter the structural or functional properties of the peptide immunogen construct. Peptide compositions can contain one or more of the disclosed peptide immunogen constructs.

b. Pharmaceutical Compositions

The present disclosure is also directed to pharmaceutical compositions containing the disclosed peptide immunogen constructs.

Pharmaceutical compositions can contain carriers and/or other additives in a pharmaceutically acceptable delivery system. Accordingly, pharmaceutical compositions can contain a pharmaceutically effective amount of one or more peptide immunogen construct together with pharmaceutically-acceptable carrier, adjuvant, and/or other excipients such as diluents, additives, stabilizing agents, preservatives, solubilizing agents, buffers, and the like.

Pharmaceutical compositions can contain one or more adjuvant that act(s) to accelerate, prolong, or enhance the immune response to the peptide immunogen constructs without having any specific antigenic effect itself. Adjuvants used in the pharmaceutical composition can include oils, aluminum salts, virosomes, aluminum phosphate (e.g., ADJU-PHOS®), aluminum hydroxide (e.g., ALHYDROGEL®), liposyn, saponin, squalene, L121, Emulsigen®, monophosphoryl lipid A(MPL), QS21, ISA 35, ISA 206, ISA50V, ISA51, ISA 720, as well as the other adjuvants and emulsifiers.

In some embodiments, the pharmaceutical compositions described herein comprise an adjuvant which is an aluminum adjuvant. In some embodiments, the adjuvant is an aluminum salt. In some embodiments, the aluminum salt is aluminum phosphate (ADJU-PHOS®) or AlPO₄. In some embodiments, the aluminum salt is aluminum hydroxide (ALHYDROGEL®) or Al(OH)₃. In some embodiments, the peptide immunogens are adsorbed to aluminum gels. In some embodiments, the peptide immunogen is precipitated with an aluminum adjuvant.

In some embodiments, the pharmaceutical composition is a mineral salt suspension. In some embodiments, the pharmaceutical composition is a gel. In some embodiments, the pharmaceutical composition is a polymeric gel. In some embodiments, the pharmaceutical composition is a wet gel suspension.

In some embodiments, the pharmaceutical composition contains MONTANIDE™ ISA 51 (an oil adjuvant composition comprised of vegetable oil and mannide oleate for production of water-in-oil emulsions), Tween® 80 (also known as: Polysorbate 80 or Polyoxyethylene (20) sorbitan monooleate), a CpG oligonucleotide, and/or any combination thereof. In other embodiments, the pharmaceutical composition is a water-in-oil-in-water (i.e., w/o/w) emulsion with Emulsigen or Emulsigen D as the adjuvant.

In some embodiments, the pharmaceutical composition described herein comprises a peptide immunogen and a CpG oligonucleotide. In some embodiments, the pharmaceutical composition comprises CpG1, CpG2, and/or CpG3. In some embodiments, the pharmaceutical composition comprises a CpG1 oligonucleotide. In some embodiments, the pharmaceutical composition comprises a CpG2 oligonucleotide. In some embodiments, the pharmaceutical composition comprises a CpG3 oligonucleotide. In some embodiments, the CpG oligonucleotide acts as a peptide immunogen stabilizer. In some embodiments, the peptide immunogen and CpG oligonucleotide are in the form of a particulate. In some embodiments, the peptide immunogen and CpG oligonucleotide are formulated as a suspension. In some embodiments, the CpG oligonucleotide is as described in WO 03/068169, which is incorporated by reference herein in its entirety and specifically as it relates to CpG oligonucleotides. In some embodiments, the CpG comprises or consists of a sequence of CpG1 (tcgtcgtttt gtcgttttgt cgttttgtcg tt), CpG2 (tcgtcgtttt gtcgttttgt cgtt), or CpG3 (tcgtcgtttt gtcgttttgt egtt). In some embodiments, the CpG oligonucleotide (e.g., CpG1, CpG2, or CpG3) is a phosphorothioate oligonucleotide. In some embodiments, CpG1 comprises the sequence of 5′ TCgTCgTTTTgTCgTTTTgTCgTTTTgTCgTT 3′, which is fully phosphorothioated. Non-limiting examples of CpG oligonucleotides are provided in Table 11.

Pharmaceutical compositions can be formulated as immediate release or sustained release formulations. Additionally, the pharmaceutical compositions can be formulated for induction of systemic, or localized mucosal, immunity through immunogen entrapment and co-administration with microparticles. Such delivery systems are readily determined by one of ordinary skill in the art.

Pharmaceutical compositions can be prepared as injectables, either as liquid solutions or suspensions. Liquid vehicles containing the peptide immunogen construct can also be prepared prior to injection. The pharmaceutical composition can be administered by any suitable mode of application, for example, i.d. (intradermally), i.v. (intravenously), i.p. (intraperitoneally), i.m. (intramuscularly), intranasally, orally, subcutaneously, etc., and in any suitable delivery device. In certain embodiments, the pharmaceutical composition is formulated for intravenous, subcutaneous, intradermal, or intramuscular administration. Pharmaceutical compositions suitable for other modes of administration can also be prepared, including oral and intranasal applications.

Pharmaceutical compositions can also be formulated in a suitable dosage unit form. In some embodiments, the pharmaceutical composition contains from about 0.5 μg to about 1 mg of the peptide immunogen construct(s) per kg body weight. Effective doses of the pharmaceutical compositions vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. When delivered in multiple doses, the pharmaceutical compositions may be conveniently divided into an appropriate amount per dosage unit form. The administered dosage will depend on the age, weight and general health of the subject as is well known in the therapeutic arts.

In some embodiments, the pharmaceutical composition contains more than one peptide immunogen construct. A pharmaceutical composition containing a mixture of more than one peptide immunogen construct to allow for synergistic enhancement of the immunoefficacy of the constructs. Pharmaceutical compositions containing more than one peptide immunogen construct can be more effective in a larger genetic population due to a broad MHC class II coverage thus provide an improved immune response to the peptide immunogen constructs.

In some embodiments, the pharmaceutical composition contains a Tau peptide immunogen construct shown in Table 2, or a homologue, analogue and/or combinations thereof. In some embodiments, pharmaceutical compositions contain a Tau peptide immunogen construct including a Tau peptide from one of Tables 1 and 7-10. In some embodiments, the pharmaceutical composition contains at least two (e.g., 2 or 3) Tau peptide immunogen constructs having a B cell epitope shown in Table 1. In some embodiments, the pharmaceutical composition contains at least two (e.g., 2 or 3) Tau peptide immunogen constructs shown in Table 2. In some embodiments, the peptide immunogen constructs targeting Tau target different regions of Tau (e.g., the N-terminal region of Tau for one peptide immunogen construct and the repeat domain for the second peptide immunogen construct; e.g., SEQ ID NOs: 10 and 15, respectively).

In some embodiments, the pharmaceutical composition contains an amyloid beta peptide immunogen construct shown in Table 4, or a homologue, analogue and/or combinations thereof. In some embodiments, pharmaceutical compositions contain an amyloid beta peptide immunogen construct including an amyloid beta peptide shown in Table 3.

In some embodiments, the pharmaceutical composition contains a Tau peptide immunogen construct (or a homologue, analogue and/or combinations thereof) and an amyloid beta peptide immunogen construct (or a homologue, analogue and/or combinations thereof). In some embodiments, the pharmaceutical composition contains at least one (or e.g., 2 or 3) Tau peptide immunogen construct having a B cell epitope shown in Table 1, and at least one (or e.g., 2 or 3) amyloid beta peptide immunogen construct having a B cell epitope shown in Table 3. In some embodiments, the pharmaceutical composition contains at least one (or e.g., 2 or 3) Tau peptide immunogen construct shown in Table 2, and at least one (or e.g., 2 or 3) amyloid beta peptide immunogen construct shown in Table 4. Pharmaceutical compositions containing a peptide immunogen construct can be used to elicit an immune response and produce antibodies in a host upon administration.

c. Immunostimulatory Complexes

The present disclosure is also directed to pharmaceutical compositions containing one or more peptide immunogen constructs in the form of an immunostimulatory complex with a CpG oligonucleotide. Such immunostimulatory complexes are specifically adapted to act as an adjuvant and as a peptide immunogen stabilizer. The immunostimulatory complexes are in the form of a particulate, which can efficiently present the peptide immunogen to the cells of the immune system to produce an immune response. The immunostimulatory complexes may be formulated as a suspension for parenteral administration. The immunostimulatory complexes may also be formulated in the form of w/o emulsions, as a suspension in combination with a mineral salt or with an in-situ gelling polymer for the efficient delivery of the peptide immunogen to the cells of the immune system of a host following parenteral administration.

The stabilized immunostimulatory complex can be formed by complexing a peptide immunogen construct with an anionic molecule, oligonucleotide, polynucleotide, or combinations thereof via electrostatic association. The stabilized immunostimulatory complex may be incorporated into a pharmaceutical composition as an immunogen delivery system.

In certain embodiments, the peptide immunogen constructs are designed to contain a cationic portion that is positively charged at a pH in the range of 5.0 to 8.0. The net charge on the cationic portion of the peptide immunogen construct, or mixture of constructs, is calculated by assigning a +1 charge for each lysine (K), arginine (R) or histidine (H), a −1 charge for each aspartic acid (D) or glutamic acid (E) and a charge of 0 for the other amino acid within the sequence. The charges are summed within the cationic portion of the peptide immunogen construct and expressed as the net average charge. A suitable peptide immunogen has a cationic portion with a net average positive charge of +1. Preferably, the peptide immunogen has a net positive charge in the range that is larger than +2. In some embodiments, the cationic portion of the peptide immunogen construct is the heterologous spacer. In certain embodiments, the cationic portion of the peptide immunogen construct has a charge of +4 when the spacer sequence is (α, ε-N)Lys, ε-N-Lys-Lys-Lys-Lys (SEQ ID NO: 62).

An “anionic molecule” as described herein refers to any molecule that is negatively charged at a pH in the range of 5.0-8.0. In certain embodiments, the anionic molecule is an oligomer or polymer. The net negative charge on the oligomer or polymer is calculated by assigning a −1 charge for each phosphodiester or phosphorothioate group in the oligomer. A suitable anionic oligonucleotide is a single-stranded DNA molecule with 8 to 64 nucleotide bases, with the number of repeats of the CpG motif in the range of 1 to 10. Preferably, the CpG immunostimulatory single-stranded DNA molecules contain 18-48 nucleotide bases, with the number of repeats of CpG motif in the range of 3 to 8.

More preferably the anionic oligonucleotide is represented by the formula: 5′ X¹CGX² 3′ wherein C and G are unmethylated; and X¹ is selected from A (adenine), G (guanine) and T (thymine); and X² is C (cytosine) or T (thymine). Or, the anionic oligonucleotide is represented by the formula: 5′ (X³)₂CG(X⁴)₂ 3′ wherein C and G are unmethylated; and X³ is selected from A, T or G; and X⁴ is C or T.

The resulting immunostimulatory complex is in the form of particles with a size typically in the range from 1-50 microns and is a function of many factors including the relative charge stoichiometry and molecular weight of the interacting species. The particulated immunostimulatory complex has the advantage of providing adjuvantation and upregulation of specific immune responses in vivo. Additionally, the stabilized immunostimulatory complex is suitable for preparing pharmaceutical compositions by various processes including water-in-oil emulsions, mineral salt suspensions and polymeric gels.

### Antibodies

Also provided herein are isolated antibodies, or epitope-binding fragments thereof, that specifically bind to the B cell epitope of the disclosed peptide immunogens. In some embodiments, such antibodies are IgG antibodies. In some embodiments, such antibodies are IgG1 antibodies.

Antibodies for use in therapy can be generated using standard methods in the art and include, e.g., monoclonal antibodies, polyclonal antibodies, multispecific antibodies (e.g., bispecific and trispecific antibodies), and antibody fragments, provided that the desired antigen-binding activity and specificity is maintained. Antibody fragments include, for example, Fv, single-chain Fv (scFv), Fab, Fab′, di-scFv, sdAb (single domain antibody), and (Fab′)₂ (including a chemically linked F(ab′)₂). Antibodies also include, e.g., chimeric antibodies, humanized antibodies, and antibodies of various species such as mouse, human, cynomolgus monkey, etc.

In certain embodiments, provided herein are methods of making antibodies, or epitope-binding fragments thereof, that specifically bind to the B cell epitope of the disclosed peptide immunogens. In some embodiments, such methods comprise administering (e.g., parenterally, e.g., intramuscularly) a peptide immunogen to a subject (e.g., a mammal, such as a mouse, a rat, a non-human primate, or a human). In some embodiments, such methods further comprise, after a period of time sufficient to generate antibodies (e.g., at from about week 1 to about week 16 post-administration, or about week 6, week 9 and/or week 12 post-administration), recovering and isolating produced antibodies.

In certain embodiments, the present disclosure provides antibodies elicited by any of the Tau peptide immunogen constructs.

The disclosed Tau peptide immunogen constructs, comprising a Tau fragment, heterologous Th epitope, and optional heterologous spacer, are capable of eliciting an immune response and the production of anti-Tau antibodies when administered to a subject. The design of the Tau peptide immunogen constructs can break tolerance to self Tau and elicit the production of site-specific antibodies that recognize conformational, not linear, epitopes.

The antibodies produced by the Tau peptide immunogen constructs recognize and bind to Tau in the forms of monomers, dimers, trimers, and oligomers.

Antibodies elicited by the Tau peptide immunogen constructs surprisingly can prevent aggregation of Tau (anti-aggregation activity) and can disassociate preformed Tau aggregates (disaggregation activity).

The resulting immune responses from animals immunized with Tau peptide immunogen constructs of the present invention demonstrated the ability of the constructs to produce potent site-directed antibodies that are reactive with Tau in the forms of monomers, dimers, trimers, and oligomers.

In certain embodiments, the present disclosure provides antibodies elicited by any of the amyloid beta peptide immunogen constructs. The disclosed amyloid beta peptide immunogen constructs, comprising an amyloid beta fragment, heterologous Th epitope, and optional heterologous spacer, are capable of eliciting an immune response and the production of anti-amyloid beta antibodies when administered to a subject.

### Kits

In certain embodiments, also provided herein are kits comprising, essentially consisting of, or consisting of one, two, three or more peptide immunogen constructs described herein (e.g., including two or more Tau peptide immunogen constructs, or a Tau peptide immunogen construct and an amyloid beta immunogen construct), optionally one or more adjuvants described herein (e.g., one or more aluminum salts described herein), optionally a CpG oligonucleotide described herein, and optionally one or more excipients. In some embodiments, such kits comprise a single container comprising two, three or more peptide immunogen constructs described herein in one formulation (e.g., including two or more Tau peptide immunogen constructs, or a Tau peptide immunogen construct and an amyloid beta immunogen construct). In some embodiments, such kits comprise these components in separate containers. In some embodiments, the kits further comprise instructions for use, such as instructions regarding making a peptide immunogen composition using the provided components. In some embodiments, the kits further comprise a delivery vehicle (e.g., a syringe) for administering a peptide immunogen composition to a subject.

### Methods

The present disclosure is also directed to methods for making and using the peptide immunogen constructs, compositions, and pharmaceutical compositions.

a. Methods for Manufacturing the Peptide Immunogen Constructs

The peptide immunogen constructs of this disclosure can be made by chemical synthesis methods well known to the ordinarily skilled artisan (see, e.g., Fields et al., Chapter 3 in Synthetic Peptides: A User's Guide, ed. Grant, W. H. Freeman & Co., New York, NY, 1992, p. 77). The peptide immunogen constructs can be synthesized using the automated Merrifield techniques of solid phase synthesis with the α-NH₂ protected by either t-Boc or F-moc chemistry using side chain protected amino acids on, for example, an Applied Biosystems Peptide Synthesizer Model 430A or 431. Preparation of peptide immunogen constructs comprising combinatorial library peptides for Th epitopes can be accomplished by providing a mixture of alternative amino acids for coupling at a given variable position.

After complete assembly of the desired peptide immunogen construct, the resin can be treated according to standard procedures to cleave the peptide from the resin and the functional groups on the amino acid side chains can be deblocked. The free peptide can be purified by HPLC and characterized biochemically, for example, by amino acid analysis or by sequencing. Purification and characterization methods for peptides are well known to one of ordinary skill in the art. The quality of peptides produced by this chemical process can be controlled and defined and, as a result, reproducibility of peptide immunogen constructs, immunogenicity, and yield can be assured.

The range in structural variability that allows for retention of an intended immunological activity has been found to be far more accommodating than the range in structural variability allowed for retention of a specific drug activity by a small molecule drug or the desired activities and undesired toxicities found in large molecules that are co-produced with biologically-derived drugs. Thus, peptide analogues, either intentionally designed or inevitably produced by errors of the synthetic process as a mixture of deletion sequence byproducts that have chromatographic and immunologic properties similar to the intended peptide, are frequently as effective as a purified preparation of the desired peptide. Designed analogues and unintended analogue mixtures are effective as long as a discerning QC procedure is developed to monitor both the manufacturing process and the product evaluation process so as to guarantee the reproducibility and efficacy of the final product employing these peptides.

The peptide immunogen constructs can also be made using recombinant DNA technology including nucleic acid molecules, vectors, and/or host cells. As such, nucleic acid molecules encoding the peptide immunogen construct and immunologically functional analogues thereof are also encompassed by the present disclosure as part of the present invention. Similarly, vectors, including expression vectors, comprising nucleic acid molecules as well as host cells containing the vectors are also encompassed by the present disclosure as part of the present invention.

Various exemplary embodiments also encompass methods of producing the peptide immunogen construct and immunologically functional analogues thereof. For example, methods can include a step of incubating a host cell containing an expression vector containing a nucleic acid molecule encoding a peptide immunogen construct and/or immunologically functional analogue thereof under such conditions where the peptide and/or analogue is expressed. The longer synthetic peptide immunogens can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

b. Methods for the Manufacturing of Immunostimulatory Complexes

Various exemplary embodiments also encompass methods of producing the immunostimulatory complexes comprising peptide immunogen constructs and CpG oligodeoxynucleotide (ODN) molecule. Stabilized immunostimulatory complexes (ISC) are derived from a cationic portion of the peptide immunogen constructs and a polyanionic CpG ODN molecule. The self-assembling system is driven by electrostatic neutralization of charge. Stoichiometry of the molar charge ratio of cationic portion of the peptide immunogen constructs to anionic oligomer determines extent of association. The non-covalent electrostatic association of peptide immunogen constructs and CpG ODN is a completely reproducible process. The peptide/CpG ODN immunostimulatory complex aggregates, which facilitate presentation to the “professional” antigen-presenting cells (APC) of the immune system thus further enhancing the immunogenicity of the complexes. These complexes are easily characterized for quality control during manufacturing. The peptide/CpG ISC are well tolerated in vivo. The particulate system comprising CpG ODN and B cell epitope-containing fragment derived peptide immunogen constructs was designed to take advantage of the generalized B cell mitogenicity associated with CpG ODN use, yet promote balanced Th-1/Th-2 type responses.

The CpG ODN in the disclosed pharmaceutical compositions is 100% bound to immunogen in a process mediated by electrostatic neutralization of opposing charge, resulting in the formation of micron-sized particulates. The particulate form allows for a significantly reduced dosage of CpG from the conventional use of CpG adjuvants, less potential for adverse innate immune responses, and facilitates alternative immunogen processing pathways including antigen-presenting cells (APC). Consequently, such formulations are novel conceptually and offer potential advantages by promoting the stimulation of immune responses by alternative mechanisms.

c. Methods for the Manufacturing of Pharmaceutical Compositions

Various exemplary embodiments also encompass pharmaceutical compositions containing peptide immunogen constructs described herein. In certain embodiments, the pharmaceutical compositions employ water in oil emulsions or mineral salt suspensions.

In order for a pharmaceutical composition to be used by a large population and with prevention of Tau or amyloid aggregation also being part of the goal for administration, safety becomes another important factor for consideration. Despite the use of water-in-oil emulsions in humans for many formulations in clinical trials, Alum remains the major adjuvant for use in formulations due to its safety. Alum or its mineral salt Aluminum phosphate (ADJUPHOS) are, therefore, frequently used as adjuvants in preparation for clinical applications.

Other adjuvants and immunostimulating agents that can be used include 3 De-O-acylated monophosphoryl lipid A (MPL) or 3-DMP, polymeric or monomeric amino acids, such as polyglutamic acid or polylysine. Such adjuvants can be used with or without other specific immunostimulating agents, such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′ dipalmitoyl-sn-glycero-3-hydroxyphosphoryloxy)-ethylamine (MTP-PE), N-acetylglucsaminyl-N-acetylmuramyl-L-AI-D-isoglu-L-Ala-dipalmitoxy propylamide (DTP-DPP) Theramide™), or other bacterial cell wall components. Oil-in-water emulsions include MF59 (see WO 90/14837 to Van Nest et al., which is hereby incorporated by reference in its entirety), containing 5% Squalene, 0.5% Tween 80, and 0.5% Span 85 (optionally containing various amounts of MTP-PE) formulated into submicron particles using a microfluidizer; SAF, containing 10% Squalene, 0.4% Tween 80, 5% pluronic-blocked polymer L121, and thr-MDP, either microfluidized into a submicron emulsion or vortexed to generate a larger particle size emulsion; and the Ribi™ adjuvant system (RAS) (Ribi ImmunoChem, Hamilton, Mont.) containing 2% squalene, 0.2% Tween 80, and one or more bacterial cell wall components selected from monophosphoryllipid A (MPL), trehalose dimycolate (TDM), and cell wall skeleton (CWS), preferably MPL+CWS (Detox™). Other adjuvants include Complete Freund's Adjuvant (CFA), Incomplete Freund's Adjuvant (IFA), and cytokines, such as interleukins (IL-1, IL-2, and IL-12), granulocyte-macrophage colony stimulating factor (GM-CSF), and tumor necrosis factor (TNF).

The choice of an adjuvant depends on the stability of the immunogenic formulation containing the adjuvant, the route of administration, the dosing schedule, the efficacy of the adjuvant for the species being vaccinated, and, in humans, a pharmaceutically acceptable adjuvant is one that has been approved or is approvable for human administration by pertinent regulatory bodies. For example, alum, MPL or Incomplete Freund's adjuvant (Chang et al., Advanced Drug Delivery Reviews 32:173-186 (1998), which is hereby incorporated by reference in its entirety) alone or optionally all combinations thereof are suitable for human administration.

The compositions can include pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, non-immunogenic stabilizers, and the like.

Pharmaceutical compositions can also include large, slowly metabolized macromolecules, such as proteins, polysaccharides like chitosan, polylactic acids, polyglycolic acids and copolymers (e.g., latex functionalized sepharose, agarose, cellulose, and the like), polymeric amino acids, amino acid copolymers, and lipid aggregates (e.g., oil droplets or liposomes). Additionally, these carriers can function as immunostimulating agents (i.e., adjuvants).

The pharmaceutical compositions of the present invention can further include a suitable delivery vehicle. Suitable delivery vehicles include, but are not limited to viruses, bacteria, biodegradable microspheres, microparticles, nanoparticles, liposomes, collagen minipellets, and cochleates.

d. Methods Using Pharmaceutical Compositions

The present disclosure also includes methods of using pharmaceutical compositions containing Tau peptide immunogen constructs, and methods of using pharmaceutical compositions containing one or more Tau peptide immunogen construct and/or one or more Aβ peptide immunogen construct.

In some embodiments, the methods employ one or more compositions including one or more Tau peptide immunogen construct, and one or more separate compositions comprising one or more Aβ peptide immunogen construct. In some embodiments, one or more Tau peptide immunogen construct and one or more Aβ peptide immunogen construct are within a single composition.

In some embodiments, the methods employ one, two or more compositions including two or more Tau peptide immunogen constructs. In some embodiments, the two or more Tau peptide immunogen constructs and in separate compositions. In some embodiments, the two or more Tau peptide immunogen constructs are within a single composition.

In certain embodiments, the pharmaceutical compositions containing the peptide immunogen constructs can be used for:
(a) inhibiting Tau aggregation in a host;
(b) inducing disaggregate of preformed Tau aggregates a host;
(c) reducing neurodegeneration triggered by exogeneous Tau aggregates in a host;
(d) reducing neurodegeneration in Tau overexpressing cells;
(e) reducing serum Tau levels in a host;
(f) reducing oligomeric Tau level in the brain of a host;
(g) reducing neuropathology and recovery of motor activities in a host; and the like;
(h) reducing amyloid plaque formation.

In some embodiments, administration to a subject (e.g., a mammal) of the Tau peptide immunogen construct or any combination composition described herein reduces, inhibits or prevents aggregation (e.g., pathological aggregation) of Tau in the brain of the subject.

In some embodiments, administration to a subject (e.g., a mammal) of the Tau peptide immunogen construct or any combination composition described herein reduces, inhibits or prevents formation of neurofibrillary tangles in the brain of the subject.

In some embodiments, administration to a subject (e.g., a mammal) of the Tau peptide immunogen construct or any combination composition described herein reduces the level of oligomeric Tau in the brain of the subject.

In some embodiments, administration to a subject (e.g., a mammal) of the Tau peptide immunogen construct or any combination composition described herein reduces the level of Tau fibrils in the brain of the subject. In some embodiments, the Tau fibrils comprise p301S mutation. In some embodiments, the Tau fibrils comprise p301L mutation. In some embodiments, administration to a subject (e.g., a mammal) of the Tau peptide immunogen construct or any combination composition described herein reduces the level of Tau carrying p301S or p301L mutation (e.g., fibril Tau) in the brain of the subject.

In some embodiments, administration of the Tau peptide immunogen construct or any combination composition described herein to a subject (e.g., a mammal) reduces monomeric Tau levels in the serum or the brain of the subject.

In some embodiments, administration to a subject (e.g., a mammal) of the amyloid beta peptide immunogen construct or any combination composition described herein reduces, inhibits or prevents accumulation of amyloid aggregates comprising amyloid beta peptide in the brain of the subject.

In some embodiments, administration to a subject (e.g., a mammal) of the amyloid beta peptide immunogen construct described herein reduces, inhibits or prevents amyloid plaque formation in the brain of the subject.

In some embodiments, administration to a subject (e.g., a mammal) of the amyloid beta peptide immunogen construct or any combination composition described herein reduces the level of oligomeric amyloid beta in the brain of the subject.

In some embodiments, administration to a subject (e.g., a mammal) of the amyloid beta peptide immunogen construct or any combination composition described herein reduces the level of amyloid beta fibrils in the brain of the subject.

The methods comprise administering a pharmaceutical composition comprising a pharmacologically effective amount of one or more peptide immunogen construct to a subject in need thereof.

In some embodiments, the methods described herein are for the treatment and/or prevention of a tauopathy.

As used herein a “tauopathy” encompasses any neurodegenerative disease that involves the pathological aggregation of the microtubule protein Tau within the brain. Accordingly, in addition to both familial and sporadic Alzheimer's disease, other tauopathies that can be treated using the methods of the present invention include, without limitation, frontotemporal dementia, parkinsonism linked to chromosome 17 (FTDP-17), progressive supranuclear palsy, corticobasal degeneration. Pick's disease, progressive subcortical gliosis, tangle only dementia, diffuse neurofibrillary tangles with calcification, argyrophilic grain dementia, amyotrophic lateral sclerosis parkinsonism-dementia complex, dementia pugilistica, Down syndrome, Gerstmann-Straussler-Scheinker disease, Hallerworden-Spatz disease, inclusion body myositis. Creutzfeld-Jakob disease, multiple system atropy, Niemann-Pick disease type C, prion protein cerebral amyloid angiopathy, subacute sclerosing panencephalitis, myotonic dystrophy, non-guanamian motor neuron disease with neurofibrillary tangles, postencephalitic parkinsonism, and chronic traumatic encephalopathy. The methods can further be used in the prevention and treatment of Lewy body disease.

In some embodiments, the methods described herein are for the treatment and/or prevention of dementia of the Alzheimer's type.

In some embodiments, the methods described herein are for the treatment and/or prevention of Alzheimer's disease.

In some embodiments, the methods described herein are for inhibiting formation of or reducing the level of misfolded or pathological Tau, e.g., prior to the clinical onset of Alzheimer's disease.

In some embodiments, the methods described herein are for inhibiting formation of or reducing the level of amyloid plaques, e.g., prior to the clinical onset of Alzheimer's disease.

Another aspect of the present disclosure is directed to a method of promoting clearance of Tau and/or Aβ aggregates from the brain of a subject. This method involves administering, to the subject, any one or more immunogenic Tau or Aβ peptide immunogen construct described herein, e.g., see Tables 2 and 4, or one or more antibodies recognizing an epitope of one of such peptides, under conditions effective to promote clearance of Tau or Aβ aggregates from the brain of the subject. In some embodiments, these methods include administration of one or more Tau peptide immunogen constructs and one or more Aβ peptide immunogen constructs, e.g., as described herein.

The clearance of Tau aggregates includes clearance of neurofibrillary tangles and/or the pathological Tau precursors to neurofibrillary tangles. Neurofibrillary tangles are often associated with neurodegenerative diseases including, for example, sporadic and familial Alzheimer's disease, amyotrophic lateral sclerosis, argyrophilic grain dementia, dementia pugilistica, chronic traumatic encephalopathy, diffuse neurofibrillary tangles with calcification, Down syndrome, Gerstmann-Straussler-Scheinker disease, Hallervorden-Spatz disease, hereditary frontotemporal dementia, parkinsonism linked to chromosome 17 (FTDP-17), inclusion body myositis, Creutsfeld-Jakob disease, multiple system atrophy, Niemann-Pick disease type C, Pick's disease, prion protein cerebral amyloid angiopathy, sporadic corticobasal degeneration, progressive supranuclear palsy, subacute sclerosing panencephalitis, myotonic dystrophy, motor neuron disease with neurofibrillary tangles, tangle only dementia, and progressive subcortical gliosis.

Another aspect of the present disclosure is directed to a method of slowing the progression of a Tau- or Aβ-pathology related behavioral phenotype in a subject. This method involves administering, to the subject, any one or more immunogenic Tau or Aβ peptide immunogenic constructs described herein, e.g., see Tables 2 and 4, or one or more antibodies recognizing an immunogenic epitope of one of such peptides, under conditions effective to slow the Tau- or Aβ-pathology related behavioral phenotype in the subject. This method can also involve administration of one or more Tau peptide immunogen constructs and one or more Aβ peptide immunogen construct, e.g., as described herein.

As used herein, a Tau-pathology or Aβ-pathology related behavioral phenotype includes, without limitation, cognitive impairments, early personality change and disinhibition, apathy, abulia, mutism, apraxia, perseveration, stereotyped movements/behaviors, hyperorality, disorganization, inability to plan or organize sequential tasks, selfishness/callousness, antisocial traits, a lack of empathy, halting, agrammatic speech with frequent paraphasic errors but relatively preserved comprehension, impaired comprehension and word-finding deficits, slowly progressive gait instability, retropulsions, freezing, frequent falls, non-levodopa responsive axial rigidity, supranuclear gaze palsy, square wave jerks, slow vertical saccades, pseudobulbar palsy, limb apraxia, dystonia, cortical sensory loss, and tremor.

In accordance with the methods of the present disclosure, in one embodiment, an immunogenic Tau peptide, a combination of immunogenic Tau peptides, or a combination of one or more Tau peptide immunogen construct and one or more Aβ immunogen construct are administered to a subject in need. In some embodiments, the subject (e.g., a human) has the disease, condition or pathology described herein or treated in accordance with the methods provided herein (e.g., has been diagnosed with such disease, condition or pathology). In some embodiments, the subject (e.g., a human) is at risk of the disease, condition or pathology described herein.

Suitable immunogenic peptide fragments of the protein contain one or more antigenic epitopes that mimic the pathological form of the corresponding protein. Exemplary immunogenic Tau epitopes are phosphorylated at one or more amino acids that are phosphorylated in the pathological form of Tau, but not phosphorylated in the normal or non-pathological form of Tau.

In some embodiments, administration of an immunogenic Tau or Aβ peptide induces an active immune response in the subject to the immunogenic Tau or Aβ peptide and to the pathological form of Tau or Aβ, thereby facilitating the clearance of related Tau or Aβ aggregates, slowing the progression of Tau- or Aβ-pathology related behavior and treating the underlying disease. In accordance with this aspect of the present invention, an immune response involves the development of a beneficial humoral (antibody mediated) and/or a cellular (mediated by antigen-specific T cells or their secretion products) response directed against the immunogenic Tau or Aβ peptide.

The presence of a humoral immunological response can be determined and monitored by testing a biological sample (e.g., blood, plasma, serum, urine, saliva feces, CSF or lymph fluid) from the subject for the presence of antibodies directed to the immunogenic Tau or Aβ peptide. Methods for detecting antibodies in a biological sample are well known in the art, e.g., ELISA, Dot blots, SDS-PAGE gels or ELISPOT. The presence of a cell-mediated immunological response can be determined by proliferation assays (CD4+ T cells) or CTL (cytotoxic T lymphocyte) assays which are readily known in the art.

In some embodiments of any of the methods described herein, the administration is parenteral administration. In some embodiments, the administration is intravenous (IV), subcutaneous (SC), intramuscular (IM), intradermal (ID), or intraperitoneal (IP) administration. In some embodiments, the administration is intramuscular. In other embodiments, the administration is intranasal.

Isolated immunogenic Tau peptides of the present invention that can be used in Tau peptide immunogen constructs in any of the methods described herein include any one of the amino acid sequences of Tables 1 and 7-10 below.

Isolated immunogenic Aβ peptides that can be used in amyloid beta peptide immunogen constructs in any of the methods described herein include any one of the amino acid sequences of Table 3 and any described in WO 2014/143087 and/or U.S. Pat. No. 9,102,752 B2 (including peptides within the formulae thereof).

### Specific Numbered Embodiments 1

Specific embodiments of the disclosure are set forth in the following numbered paragraphs.

1. A Tau peptide immunogen construct represented by the formulae:

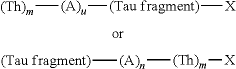

wherein
Th is a heterologous T helper epitope;
A is a heterologous spacer;
(Tau fragment) is a B cell epitope having about 10 to about 40 amino acid residues from the full-length Tau protein of SEQ ID NO: 60;
X is an α-COOH or α-CONH₂ of an amino acid;
m is from 1 to about 4; and
n is from 0 to about 10.

2. The Tau peptide immunogen construct according to paragraph 1, wherein the Tau fragment is selected from SEQ ID NOs: 1-8, 17-30, and 63-67.

3. The Tau peptide immunogen construct according to any one of paragraphs 1 or 2, wherein the Th epitope is SEQ ID NOs: 31-59.

4. The Tau peptide immunogen construct according to paragraph 1, wherein the peptide immunogen construct is selected from SEQ ID NOs: 9-16 or a construct in which the positions of the Tau peptide and the Th peptide are reversed.

5. A Tau peptide immunogen construct comprising:
a B cell epitope comprising about 8 to about 40 amino acid residues from the full-length Tau protein sequence of SEQ ID NO: 60 (e.g., about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids);
a T helper epitope comprising an amino acid sequence selected from SEQ ID NOs: 31-59; and
an optional heterologous spacer selected from an amino acid, Lys-, Gly-, Lys-Lys-Lys-, (α, ε-N)Lys, and ε-N-Lys-Lys-Lys-Lys (SEQ ID NO: 62),
wherein the B cell epitope is covalently linked to the T helper epitope directly or through the optional heterologous spacer.

6. The Tau peptide immunogen construct of paragraph 5, wherein the B cell epitope is selected from SEQ ID NOs: 1-8, 17-30, and 63-67.

7. The Tau peptide immunogen construct of paragraph 5 or 6, wherein the T helper epitope is selected from SEQ ID NOs: 31-59.

8. The Tau peptide immunogen construct of any one of paragraphs 5 to 7, wherein the optional heterologous spacer is (α, ε-N)Lys or ε-N-Lys-Lys-Lys-Lys (SEQ ID NO: 62).

9. The Tau peptide immunogen construct of any one of paragraphs 5 to 8, wherein the T helper epitope is covalently linked to the amino terminus of the B cell epitope.

10. The Tau peptide immunogen construct of any one of paragraphs 5 to 9, wherein the T helper epitope is covalently linked to the amino terminus of the B cell epitope through the optional heterologous spacer.

11. A composition comprising a Tau peptide immunogen construct according to any one of paragraphs 1 to 10.

12. A pharmaceutical composition comprising:
a. a Tau peptide immunogen construct according to any one of paragraphs 1 to 10; and
b. and a pharmaceutically acceptable delivery vehicle and/or adjuvant.

13. The pharmaceutical composition of paragraph 12, wherein
a. the Tau peptide immunogen construct is selected from SEQ ID NOs: 9-16, or a Tau peptide immunogen construct in which the positions of the B cell epitope and the T cell epitope are reversed as compared to one of SEQ ID NOs: 9-16; and
b. the Tau peptide immunogen construct is mixed with an CpG oligodeoxynucleotide (ODN) to form a stabilized immunostimulatory complex.

14. The composition of any one of paragraphs 11-13, wherein the composition or pharmaceutical composition comprises more than one of said Tau peptide immunogen constructs.

15. The composition of any one of paragraphs 11-14, wherein the composition or pharmaceutical composition comprises 2, 3, or 4 of said Tau peptide immunogen constructs.

16. The composition of paragraph any one of paragraphs 11-15, wherein the composition or pharmaceutical composition comprises one or more of SEQ ID NOs: 9-16, SEQ ID NO: 10, SEQ ID NO: 15, SEQ ID NO: 10 and SEQ ID NO: 15, or any combination thereof, or wherein the composition or pharmaceutical composition comprises a first peptide immunogen construct targeting a first region of Tau (e.g., the N-terminal region) and a second peptide immunogen construct targeting a second region of Tau (e.g., the repeat sequence), optionally wherein these peptide immunogen constructs comprise SEQ ID NO: 2 and/or 10, and 7 and/or 15, respectively.

17. An isolated antibody or epitope-binding fragment thereof that specifically binds to the B cell epitope of the Tau peptide immunogen construct according to any one of paragraphs 1 to 10.

18. The isolated antibody or epitope-binding fragment thereof according to paragraph 17 bound to the Tau peptide immunogen construct.

19. A composition comprising the isolated antibody or epitope-binding fragment thereof according to paragraph 17 or 18.

20. A Tau peptide immunogen construct selected:

a.
SEQ ID NO: 9
MAEPRQEFEVMEDHAGTYGLGDKKK-eK-
ISITEIKGVIVHRIETILF-NH2;
b.
SEQ ID NO: 10
DHAGTYGLGDKKK-eK-ISITEIKGVIVHRIETILF-NH2;
c.
SEQ ID NO: 11
ISITEIKGVIVHRIETILF-eK-KKKNITHVPGGGNKK-NH2;
d.
SEQ ID NO: 12
ISITEIKGVIVHRIETILF-eK-KKKKDNIKHVPGGGSVQIVYK-
NH2;
e.
SEQ ID NO: 13
ISITEIKGVIVHRIETILF-eK-KKKTKIATPRGAAPP-NH2;
f.
SEQ ID NO: 14
ISITEIKGVIVHRIETILF-cK-KKKVVRTPPKSPSSAKSRL-NH2;
g.
SEQ ID NO: 15
ISITEIKGVIVHRIETILF-eK-KKKIKHVPGGGSVQIVYK-NH2;
and
h.
SEQ ID NO: 16
ISITEIKGVIVHRIETILF-eK-KKKVSGDTSPRHLSNVSST-NH2

21. A composition comprising a Tau peptide immunogen construct according to paragraph 20.

22. A pharmaceutical composition comprising:
a. a Tau peptide immunogen construct according to paragraph 20; and
b. and a pharmaceutically acceptable delivery vehicle and/or adjuvant.

23. The pharmaceutical composition of paragraph 22, wherein the Tau peptide immunogen construct is mixed with an CpG oligodeoxynucleotide (ODN) to form a stabilized immunostimulatory complex.

24. The composition of any one of paragraphs 21-23, wherein the composition or pharmaceutical composition comprises more than one of said Tau peptide immunogen constructs.

25. The composition of any one of paragraphs 21-24, wherein the composition or pharmaceutical composition comprises 2, 3, or 4 of said Tau peptide immunogen constructs.

26. The composition of any previous paragraph, wherein the composition or pharmaceutical composition further comprises one or more amyloid-beta (Aβ) peptide immunogen construct.

27. The composition of paragraph 26, wherein the composition comprises an Aβ peptide immunogen construct of SEQ ID NO: 70, an Aβ peptide immunogen construct of SEQ ID NO: 71, or an Aβ peptide immunogen construct of SEQ ID NO: 70 and an Aβ peptide immunogen construct of SEQ ID NO: 71.

28. The composition of any previous paragraph, wherein the composition comprises any one or more of SEQ ID NOs: 9-16, SEQ ID NO: 10, SEQ ID NO: 15, SEQ ID NO: 10 and SEQ ID NO: 15, or any combination thereof, for example, a combination of SEQ ID NO: 10 and SEQ ID NOs: 70 and 71, a combination of SEQ ID NO: 11 and SEQ ID NOs: 70 and 71, a combination of SEQ ID NO: 12 and SEQ ID NOs: 70 and 71, a combination of SEQ ID NO: 13 and SEQ ID NOs: 70 and 71, a combination of SEQ ID NOs: 14 and SEQ ID NOs: 70 and 71, a combination of SEQ ID NO: 15 and SEQ ID NOs: 70 and 71, a combination of SEQ ID NO: 16 and SEQ ID NOs: 70 and 71, or a combination listed above that includes SEQ ID NO: 70 but not SEQ ID NO: 71 or includes SEQ ID NO: 71 but not SEQ ID NO: 70, or wherein the composition or pharmaceutical composition comprises a first peptide immunogen construct targeting a first region of Tau (e.g., the N-terminal region) and a second peptide immunogen construct targeting a second region of Tau (e.g., the repeat sequence), optionally wherein these peptide immunogen constructs comprise SEQ ID NO: 2 and/or 10, and 7 and/or 15, respectively.

29. The composition of any one of paragraphs 26-28, wherein the composition comprises an Aβ peptide immunogenic construct of WO 2014/143087.

30. A method of preventing, inhibiting, reducing the severity of, delaying, or treating a tauopathy in a subject, the method comprising administering a tau peptide immunogen construct or a composition of any one of the prior paragraphs to the subject.

31. The method of paragraph 30, wherein the tauopathy is selected from Alzheimer's disease, Lewy body disease, frontotemporal dementia, parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, dementia pugilistica, Down syndrome, Gerstmann-Straussler-Scheinker disease, Hallerworden-Spatz disease, inclusion body myositis, Creutzfeld-Jakob disease, multiple system atropy, Niemann-Pick disease type C, prion protein cerebral amyloid angiopathy, subacute sclerosing panencephalitis, myotonic dystrophy, non-guanamian motor neuron disease with neurofibrillary tangles, postencephalitic parkinsonism, and chronic traumatic encephalopathy.

32. The method of paragraph 30 or 31, wherein the tauopathy is Alzheimer's disease.

33. The method of any one of paragraphs 30-32, wherein the method comprises administration of one or more Tau peptide immunogen constructs, optionally in combination with one more amyloid beta peptide immunogen constructs.

34. The method of paragraph 33, wherein the one or more Tau peptide immunogen constructs and/or the one or more amyloid beta peptide immunogen constructs are comprised within a single composition.

35. The method of paragraph 33, wherein the one or more Tau peptide immunogen constructs and/or the one or more amyloid beta peptide immunogen constructs are comprised within separate compositions, which are administered separately.

36. The method of any one of paragraphs 30-35, comprising administration of one or more of the peptide immunogen constructs, compositions, or combinations thereof of any of the previous paragraphs.

### Specific Numbered Embodiments 2

Additional specific embodiments of the disclosure are set forth in the following numbered paragraphs.

1. A Tau peptide immunogen construct represented by the formulae:

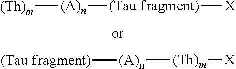

wherein
Th is a heterologous T helper epitope;
A is a heterologous spacer;
(Tau fragment) is a B cell epitope having about 10 to about 40 amino acid residues from the full-length Tau protein of SEQ ID NO: 60;
X is an α-COOH or α-CONH₂ of an amino acid;
m is from 1 to about 4; and
n is from 0 to about 10.

2. The Tau peptide immunogen construct according to paragraph 1, wherein the Tau fragment is selected from SEQ ID NOs: 1-8, 17-30, and 63-67.

3. The Tau peptide immunogen construct according to any one of paragraphs 1 or 2, wherein the Th epitope is selected from SEQ ID NOs: 31-59.

4. The Tau peptide immunogen construct according to paragraph 1, wherein the peptide immunogen construct is selected from SEQ ID NOs: 9-16 or a construct in which the positions of the Tau peptide and the Th peptide are reversed.

5. A Tau peptide immunogen construct comprising:
a B cell epitope comprising about 8 to about 40 amino acid residues from the full-length Tau protein sequence of SEQ ID NO: 60 (e.g., about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids);
a T helper epitope comprising an amino acid sequence selected from the group consisting of SEQ ID NOs: 31-59; and
an optional heterologous spacer selected from an amino acid, Lys-, Gly-, Lys-Lys-Lys-, (α, ε-N)Lys, and ε-N-Lys-Lys-Lys-Lys (SEQ ID NO: 62),
wherein the B cell epitope is covalently linked to the T helper epitope directly or through the optional heterologous spacer.

6. The Tau peptide immunogen construct of paragraph 5, wherein the B cell epitope is selected from SEQ ID NOs: 1-8, 17-30, and 63-67.

7. The Tau peptide immunogen construct of paragraph 5 or 6, wherein the T helper epitope is selected from SEQ ID NOs: 31-59.

8. The Tau peptide immunogen construct of any one of paragraphs 5 to 7, wherein the optional heterologous spacer is (α, ε-N)Lys or ε-N-Lys-Lys-Lys-Lys (SEQ ID NO: 62).

9. The Tau peptide immunogen construct of any one of paragraphs 5 to 8, wherein the T helper epitope is covalently linked to the amino terminus of the B cell epitope.

10. The Tau peptide immunogen construct of any one of paragraphs 5 to 9, wherein the T helper epitope is covalently linked to the amino terminus of the B cell epitope through the optional heterologous spacer.

11. A composition comprising a Tau peptide immunogen construct according to any one of paragraphs 1 to 10.

12. A pharmaceutical composition comprising:
a. a Tau peptide immunogen construct according to any one of paragraphs 1 to 10; and
b. and a pharmaceutically acceptable delivery vehicle and/or adjuvant.

13. The pharmaceutical composition of paragraph 12, wherein
a. the Tau peptide immunogen construct is selected from SEQ ID NOs: 9-16, or a Tau peptide immunogen construct in which the positions of the B cell epitope and the T cell epitope are reversed as compared to one of SEQ ID NOs: 9-16; and
b. the Tau peptide immunogen construct is mixed with an CpG oligodeoxynucleotide (ODN) to form a stabilized immunostimulatory complex.

14. The composition of any one of paragraphs 11-13, wherein the composition or pharmaceutical composition comprises more than one of said Tau peptide immunogen constructs.

15. The composition of any one of paragraphs 11-14, wherein the composition or pharmaceutical composition comprises 2, 3, or 4 of said Tau peptide immunogen constructs.

16. The composition of claim any one of paragraphs 11-15, wherein the composition or pharmaceutical composition comprises one or more of SEQ ID NOs: 9-16, SEQ ID NO: 10, SEQ ID NO: 15, SEQ ID NO: 10 and SEQ ID NO: 15, or any combination thereof, or wherein the composition or pharmaceutical composition comprises a first peptide immunogen construct targeting a first region of Tau (e.g., the N-terminal region) and a second peptide immunogen construct targeting a second region of Tau (e.g., the repeat sequence), optionally wherein these peptide immunogen constructs comprise SEQ ID NO: 2 and/or 10, and 7 and/or 15, respectively.

17. An isolated antibody or epitope-binding fragment thereof that specifically binds to the B cell epitope of the Tau peptide immunogen construct according to any one of paragraphs 1 to 10.

18. The isolated antibody or epitope-binding fragment thereof according to paragraph 17 bound to the Tau peptide immunogen construct.

19. A composition comprising the isolated antibody or epitope-binding fragment thereof according to paragraph 17 or 18.

20. A Tau peptide immunogen construct selected from:

a.
SEQ ID NO: 9
MAEPRQEFEVMEDHAGTYGLGDKKK-eK-
ISITEIKGVIVHRIETILF-NH2;
b.
SEQ ID NO: 10
DHAGTYGLGDKKK-eK-ISITEIKGVIVHRIETILF-NH2;
c.
SEQ ID NO: 11
ISITEIKGVIVHRIETILF-eK-KKKNITHVPGGGNKK-NH2;
d.
SEQ ID NO: 12
ISITEIKGVIVHRIETILF-eK-KKKKDNIKHVPGGGSVQIVYK-
NH2;
e.
SEQ ID NO: 13
ISITEIKGVIVHRIETILF-eK-KKKTKIATPRGAAPP-NH2;
f.
SEQ ID NO: 14
ISITEIKGVIVHRIETILF-cK-KKKVVRTPPKSPSSAKSRL-NH2;
g.
SEQ ID NO: 15
ISITEIKGVIVHRIETILF-eK-KKKIKHVPGGGSVQIVYK-NH2;
and
h.
SEQ ID NO: 16
ISITEIKGVIVHRIETILF-eK-KKKVSGDTSPRHLSNVSST-NH2

21. A composition comprising a Tau peptide immunogen construct according to paragraph 20.

22. A pharmaceutical composition comprising:
a. a Tau peptide immunogen construct according to paragraph 20; and
b. and a pharmaceutically acceptable delivery vehicle and/or adjuvant.

23. The pharmaceutical composition of paragraph 22, wherein the Tau peptide immunogen construct is mixed with an CpG oligodeoxynucleotide (ODN) to form a stabilized immunostimulatory complex.

24. The composition of any one of paragraphs 21-23, wherein the composition or pharmaceutical composition comprises more than one of said Tau peptide immunogen constructs.

25. The composition of any one of paragraphs 21-24, wherein the composition or pharmaceutical composition comprises 2, 3, or 4 of said Tau peptide immunogen constructs.

26. The composition of any previous paragraph, wherein the composition or pharmaceutical composition further comprises one or more amyloid-beta (Aβ) peptide immunogen construct.

27. The composition of paragraph 26, wherein the composition comprises an Aβ peptide immunogen construct of SEQ ID NO: 70, an Aβ peptide immunogen construct of SEQ ID NO: 71, or an Aβ peptide immunogen construct of SEQ ID NO: 70 and an Aβ peptide immunogen construct of SEQ ID NO: 71.

28. The composition of any previous paragraph, wherein the composition comprises any one or more of SEQ ID NOs: 9-16, SEQ ID NO: 10, SEQ ID NO: 15, SEQ ID NO: 10 and SEQ ID NO: 15, or any combination thereof, for example, a combination of SEQ ID NO: 10 and SEQ ID NOs: 70 and 71, a combination of SEQ ID NO: 11 and SEQ ID NOs: 70 and 71, a combination of SEQ ID NO: 12 and SEQ ID NOs: 70 and 71, a combination of SEQ ID NO: 13 and SEQ ID NOs: 70 and 71, a combination of SEQ ID NOs: 14 and SEQ ID NOs: 70 and 71, a combination of SEQ ID NO: 15 and SEQ ID NOs: 70 and 71, a combination of SEQ ID NO: 16 and SEQ ID NOs: 70 and 71, or a combination listed above that includes SEQ ID NO: 70 but not SEQ ID NO: 71 or includes SEQ ID NO: 71 but not SEQ ID NO: 70, or wherein the composition or pharmaceutical composition comprises a first peptide immunogen construct targeting a first region of Tau (e.g., the N-terminal region) and a second peptide immunogen construct targeting a second region of Tau (e.g., the repeat sequence), optionally wherein these peptide immunogen constructs comprise SEQ ID NO: 2 and/or 10, and 7 and/or 15, respectively.

29. The composition of any one of paragraphs 26-28, wherein the composition comprises an Aβ peptide immunogenic construct of WO 2014/143087.

30. A method of preventing, inhibiting, reducing the severity of, delaying, or treating a tauopathy in a subject, the method comprising administering a tau peptide immunogen construct or a composition of any one of the prior claims to the subject.

31. The method of paragraph 30, wherein the tauopathy is selected from Alzheimer's disease, Lewy body disease, frontotemporal dementia, parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, dementia pugilistica, Down syndrome, Gerstmann-Straussler-Scheinker disease, Hallerworden-Spatz disease, inclusion body myositis, Creutzfeld-Jakob disease, multiple system atropy, Niemann-Pick disease type C, prion protein cerebral amyloid angiopathy, subacute sclerosing panencephalitis, myotonic dystrophy, non-guanamian motor neuron disease with neurofibrillary tangles, postencephalitic parkinsonism, and chronic traumatic encephalopathy.

32. The method of paragraph 30 or 31, wherein the tauopathy is Alzheimer's disease.

33. The method of any one of paragraphs 30-32, wherein the method comprises administration of one or more Tau peptide immunogen constructs, optionally in combination with one more amyloid beta peptide immunogen constructs.

34. The method of paragraph 33, wherein the one or more Tau peptide immunogen constructs and/or the one or more amyloid beta peptide immunogen constructs are comprised within a single composition.

35. The method of paragraph 33, wherein the one or more Tau peptide immunogen constructs and/or the one or more amyloid beta peptide immunogen constructs are comprised within separate compositions, which are administered separately.

36. The method of any one of paragraphs 30-35, comprising administration of one or more of the peptide immunogen constructs, compositions, or combinations thereof of any of the previous claims.

### Example 1

The accumulation of misfolded Tau in the brain correlates with clinical decline in Alzheimer's Disease (AD) but manifests decades earlier than cognitive symptoms (Congdon and Sigurdsson, Nat. Rev. Neurol, 2018). Targeting pathological Tau prior to the clinical onset of AD could help prevent disease and/or progression. Described herein is a vaccine-based immunotherapy approach for the prevention and treatment of Alzheimer's Disease. The results of preclinical characterization of Tau vaccines are presented in Table A, below. The peptides noted in the Figures can be cross-referenced based on the letter code in column 1 of Table 2.

TABLE A
Immunogenicity of Tau candidates

| Lead | UbTh1 | CpG1 | Antigen | Mono | PFF |
|---|---|---|---|---|---|
| A | 10 | 107 | 396 | 504 | 17,400 |
| B | 10 | 221 | 1,490 | 3,512 | 2,060 |
| C | 10 | 199 | 2,827 | 5,732 | 745 |
| D | 17 | 18 | 10,086 | 574 | 601 |
| E | 10 | 15 | 5,216 | 554 | 498 |
| F | 10 | 39 | 97 | 2,782 | 4,050 |
| G | 10 | 59 | 4,003 | 3,299 | 4,556 |

Ec50 values generated via 12-point dilution curve ELISAs probing against the T helper peptide, CpG, Tau lead-specific peptides, full length monomeric Tau (Mono) and Tau preformed fibrils (PFF) using the terminal bleed sera (15 weeks) from vaccinated male Hartley guinea pigs.

The platform upon which the vaccines are based is illustrated in FIG. 1, and includes a B-cell epitope, a linker, and a Th peptide carrier. B-cells will recognize the B-cell epitope and generate antibodies against the peptide, which mimics the biologic target (e.g., Tau). The linker chemically links the B-cell epitope to the Th peptide, thereby optimizing the presentation of the B-cell epitope to the immune system. The Th peptide carrier activates T-helper cells, which alert B-cells to start generating antibodies against the B-cell epitope, while avoiding inflammation and off-target activity. The primary structure of Tau, with functional domains and targeting peptides annotated, is shown at the bottom of FIG. 1. The structures of the peptide immunogen constructs indicated in this figure are also shown (and referenced by letter code) in Table 2.

The peptide immunogen constructs tested as described herein were formulated in Adju-Phos CpG1 at 100 μg/mL+300 ug/0.25 mL peptide. Guinea pigs were given 5 intramuscular shots 3 weeks apart, with the terminal bleed collected at 15 wpi. Antibodies against the T helper peptide, adjuvant components, and Tau were quantified by ELISA in serial dilution. In addition, antibody binding was characterized against recombinant and brain-derived Tau preps by Western blot, dot blot, and biolayer interferometry (BLI). Assessments of in vitro function were made via Tau FRET-aggregation and pHrodo uptake assay in HEK293 and B103 cells.

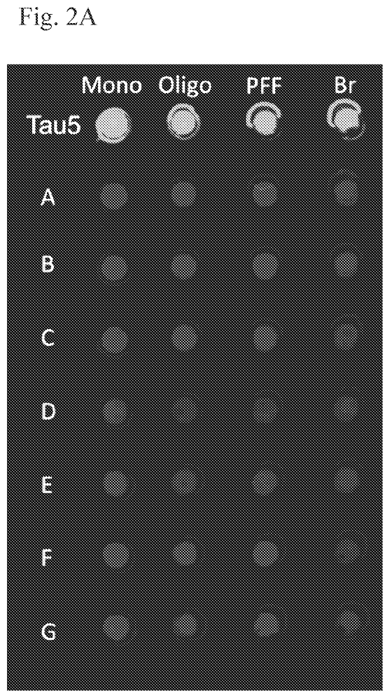

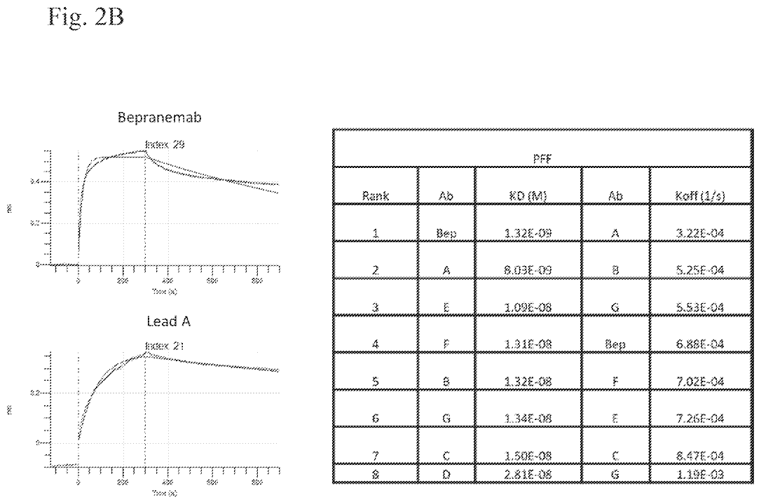

The results of antibody binding characterization are shown in FIGS. 2A and 2B. The binding potency of vaccine-induced antibodies against three forms of recombinant Tau (monomeric, oligomeric, and PFF) and a sarkosyl extract from postmortem brain tissue (Br) were characterized via dotblot (FIG. 2A). Diverse binding profiles were observed against the different forms of Tau. BLI results show antibody binding to Tau forms performing with Kd in nM range, with three Leads-induced antibodies having slower off rates than Bepranemab (FIG. 2B). Representative binding curves of Bep and Lead A-induced antibodies to PFF are presented.

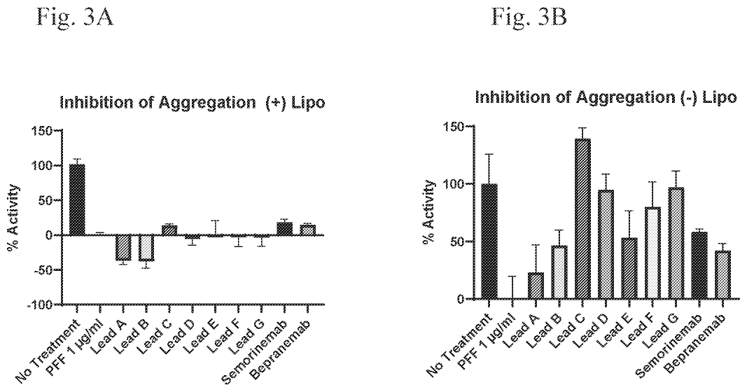

The results of an aggregation assay using a Tau biosensor line are shown in FIGS. 3A and 3B. Tau biosensor lines (Frost et al., J. Biol. Chem., 2009; 284(19):12845-12852) were used to assess the functional inhibition of Tau aggregation by vaccine induced antibodies compared to anti-Tau mAbs, Semorinemab, and Bepranemab. Assay conditions included the addition of lipofectamine (FIG. 3A) and without lipofectamine (FIG. 3B). Only the condition without lipofectamine resulted in Abs inhibiting aggregation.

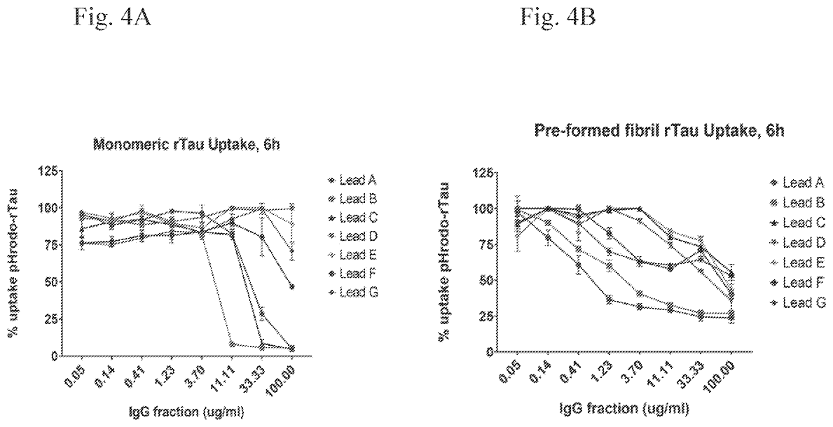

The results of a Tau uptake assay are shown in FIGS. 4A and 4B. B103 cells were exposed to pHrodo labeled Tau preparations for 6 h. Upon uptake, pHrodo-Tau fluoresces, which enabled quantification using the IncuCyte live cell imager. Leads A, B, C, and F induced antibodies that inhibited the uptake of monomeric Tau in dose responsive manner (FIG. 4A). All Leads induced antibodies resulted in a dose-dependent reduction of PFF uptake (FIG. 4B). Leads induced antibodies varied in potency; however, all exhibited stronger inhibition of PFF than monomeric Tau uptake.

The antibodies induced by the tested Tau peptide immunogen constructs displayed diverse binding profiles against different forms of Tau. No significant immunogenicity was observed against the Th1 peptides or CpG1. Functional assays illustrated that such antibodies prevented Tau aggregation (as shown by inhibition of Tau uptake).

### Example 2

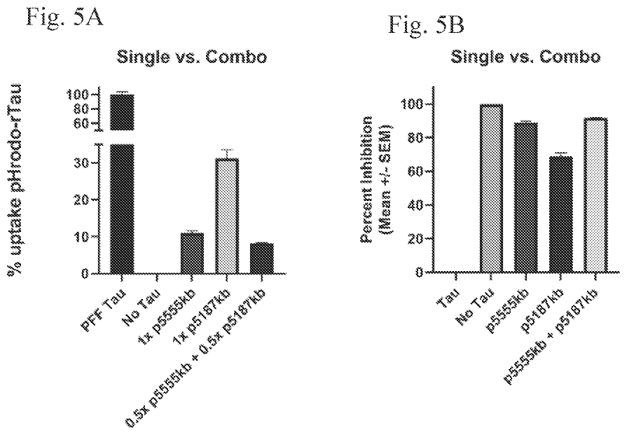

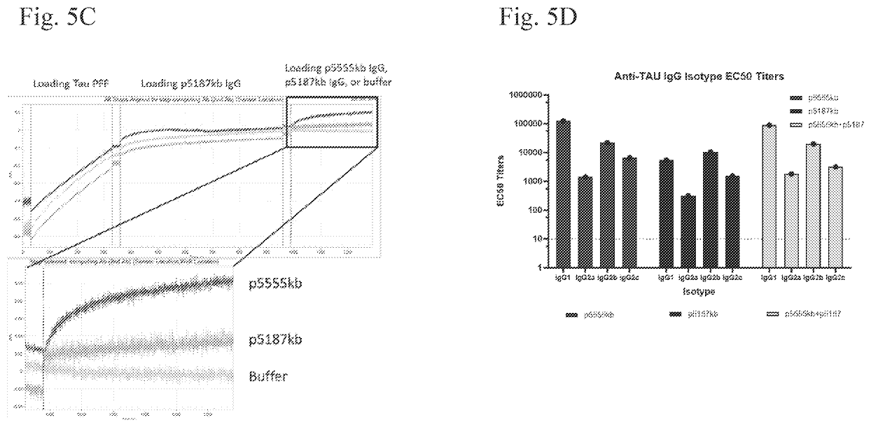

Combinations of anti-Tau IgG antibodies generated by use of p5555kb and p5187kb were tested in activity and binding assays. FIG. 5A shows the results of a Tau uptake assay in B103 cells treated with pHrodo-PFF Tau and anti-Tau antibodies from rats vaccinated with p5555kb, p5187kb, or both p5555kb and p5187kb. In FIGS. 5A, 5B and 5C, where it's p5555kb+p5187kb, the antibodies generated by the indicated vaccines were mixed together in the assay. The data show that Tau uptake is decreased when anti-Tau IgG antibodies from rats vaccinated with p5555kb and p5187kb IgGs were used. FIG. 5B is another presentation of the data in FIG. 5A, showing percent inhibition of Tau uptake.

To examine whether or not antibodies from p5555kb and p5187kb can bind the same target together, we used biolayer interferometry (BLI). Briefly, BLI allows the quantification of binding by measuring the interference of light reflected from a biosensor coated with a target protein or peptide. As binding occurs, interference increases. If two antibodies can bind together, the interference is predicted in increase following the introduction of an antibody after the binding of a different antibody to the same target. If the antibodies cannot bind the same target together, the interference would not change with a second antibody after the first antibody binds. FIG. 5C shows the results of biolayer interferometry analysis of IgGs from p5555kb and p5187kb. The data show that IgGs from p5555kb continued to bind PFF Tau following Tau binding by IgGs from p5187kb. FIG. 5D shows anti-Tau IgG isotypes generated by administration of p5555kb and p5187 kB, showing enrichment of IgG1 and IgG2b isotypes. These results support the feasibility of a combination vaccine against Tau.

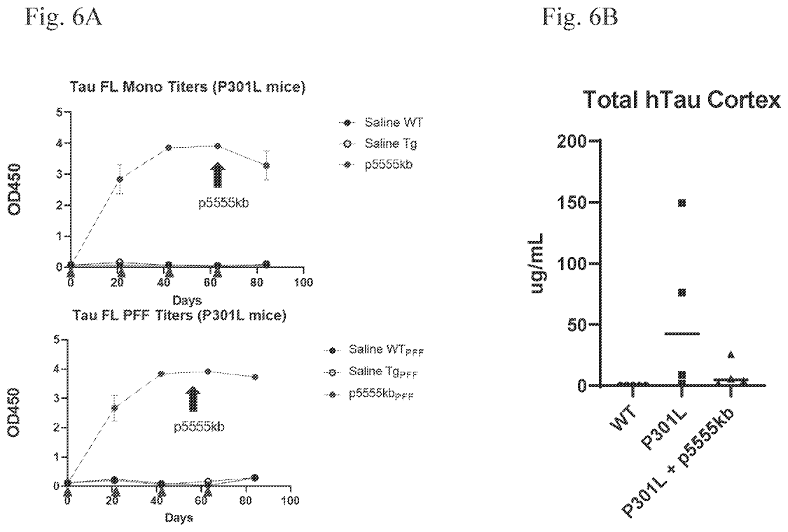

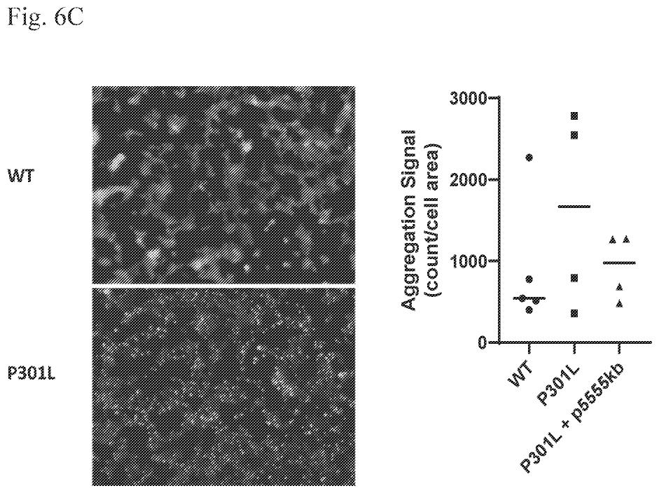

Vaccination studies in WT and P301L (mutant Tau) mice were carried out. FIG. 6A shows that P301L mice vaccinated with p5555kb generated robust titers against monomeric and PFF Tau. FIG. 6B shows that vaccination with p5555kb reduced Tau accumulation in P301L mice. FIG. 6C shows that lysates from brains of P301L mice vaccinated with p5555kb exhibited evidence of reduced induction of aggregation in a Tau biosensor line. These results show that both WT and P301L mice demonstrated positive responses to vaccination with p5555kb.

### Example 3

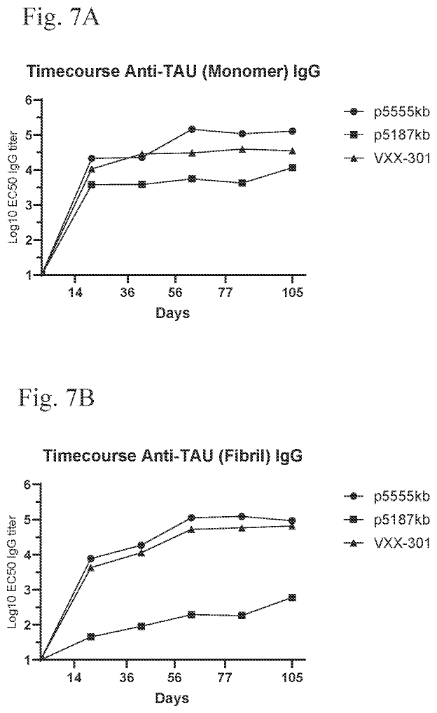

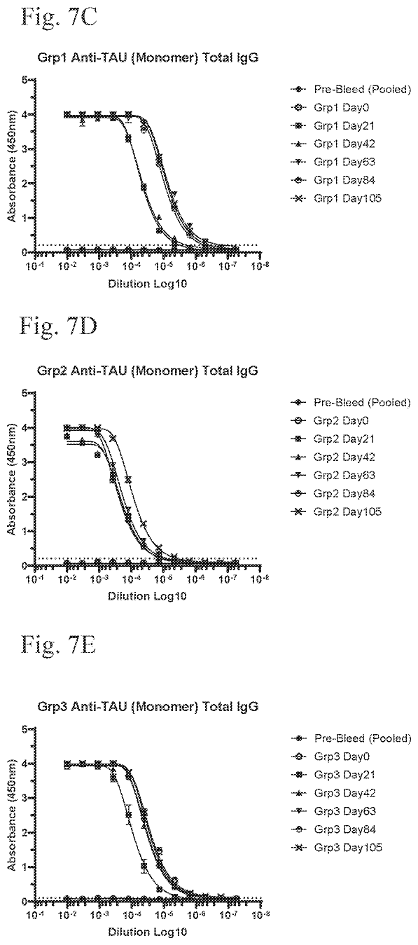

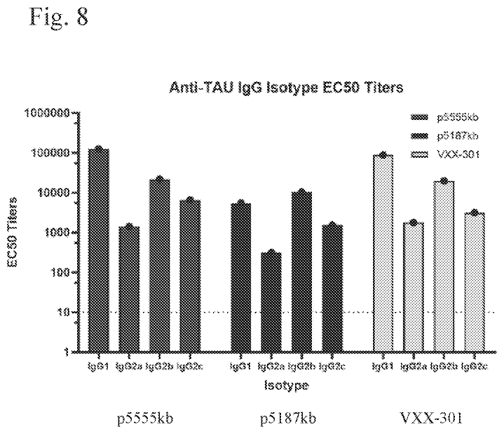

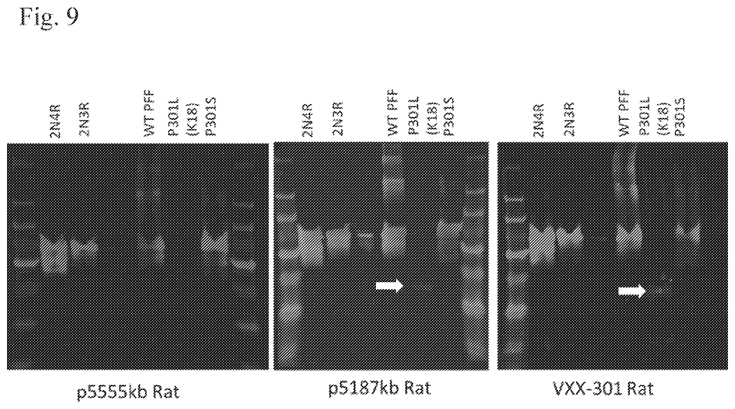

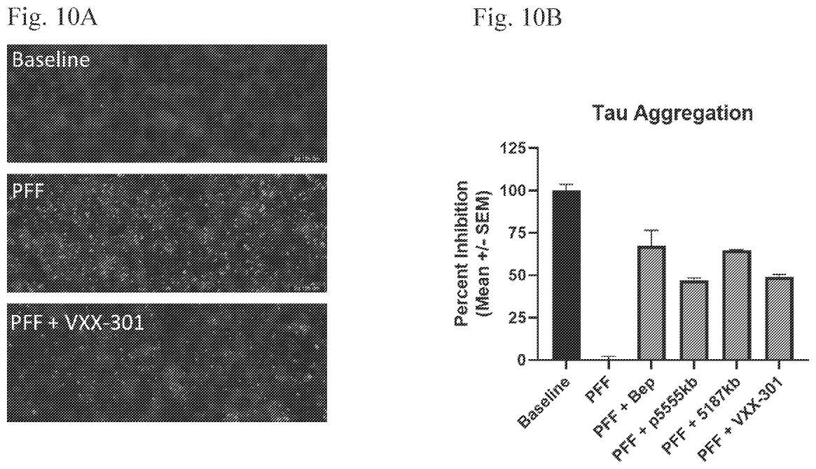

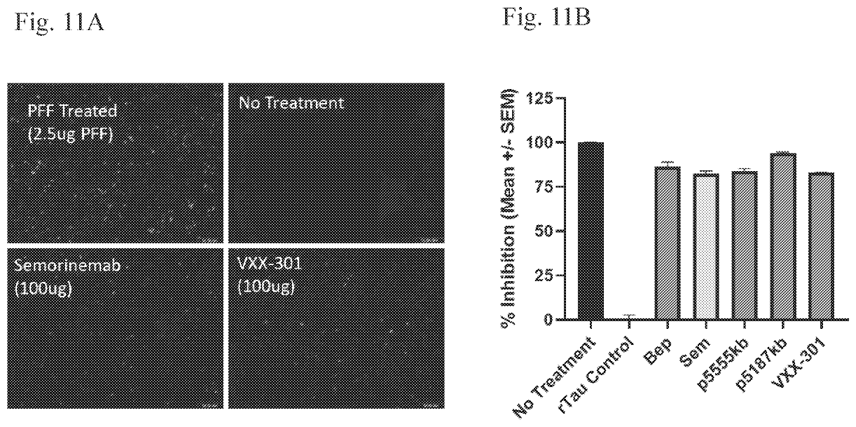

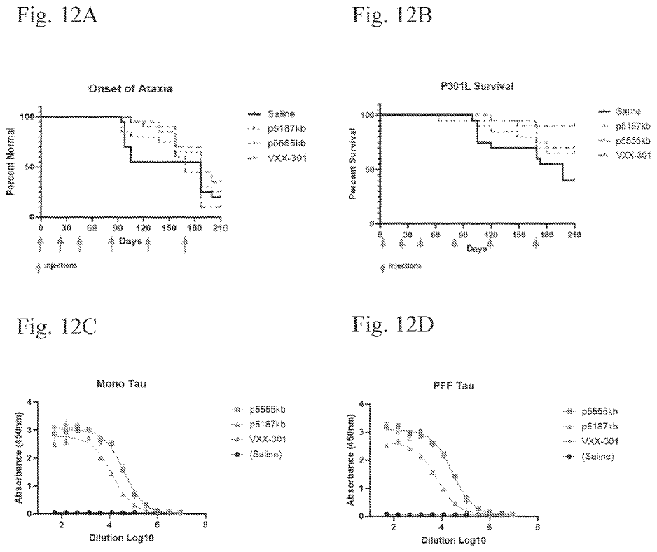

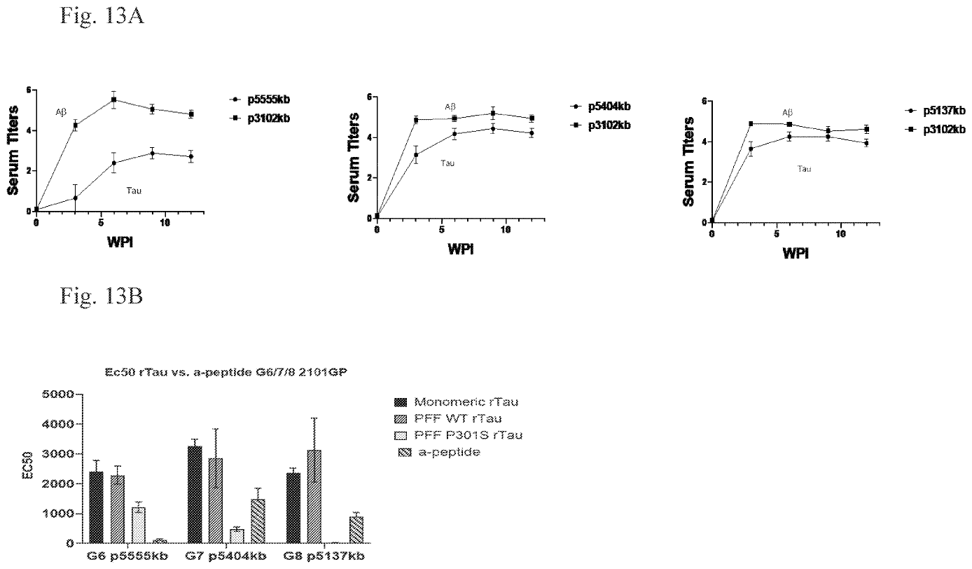

A combination Tau vaccine, VXX-301, was designed by combining p5555kb and p5187kb Tau peptide immunogen constructs in one formulation. Reference to VXX-301 in the figures refers to antibodies derived from the VXX-301 vaccinated animals. FIGS. 7A-7B show that rats vaccinated with p5555kb, p5187kb, or the combination vaccine VXX-301 generated robust antibody titers against monomeric or fibril PFF Tau. For vaccinations, 0.3 mg/ml of peptide immunogen was used per 0.25 mL dose, with 0.1 mg CpG, administered intramuscularly. FIGS. 7C-7E show that anti-monomeric Tau IgG titer curves over time following vaccination of rats with p5555kb (Grp1: FIG. 7C), p5187kb (Grp2; FIG. 7D) and VXX-301 (Grp3; FIG. 7E). FIG. 8 shows IgG isotypes of anti-Tau antibodies following vaccination with p5555kb, p5187kb, or VXX-301, showing that IgG1 and IgG2b were enriched following vaccination (and showing lower titers for IgG2a and IgG2c).

Western blot analysis of anti-Tau antibodies from rats vaccinated with p5555kb, p5187kb or VXX-301 was performed to evaluate binding to different recombinant preparations of Tau. The recombinant preparations of Tau probed for binding included 2N4R full-length Tau, 2N3R, WT preformed fibrils (PFF), and P301S mutant Tau. P301L K18 preparation is a C-terminal fragment of Tau containing the 4 repeat domain. FIG. 9 shows that p5555kb derived antibodies could bind to Tau preparations 2N4R, 2N3R. WT preformed fibrils (PFF), and P301S (but not K18), while p5187kb and VXX-301 derived antibodies could bind to all forms of Tau including P301L K18 preparation (see white arrow in FIG. 9).

Anti-Tau antibodies from vaccinated rats inhibited Tau aggregation. FIG. 10A shows that lysates from brains of rats vaccinated with VXX-301 exhibited evidence of enhanced inhibition of Tau aggregation in a Tau biosensor line. FIG. 10B shows that antibodies from p5555kb, p5187kb and VXX-301 vaccinated rats could comparably inhibit Tau aggregation when compared to Bepranemab. FIGS. 11A-11B show the results of a Tau uptake assay, showing that anti-Tau antibodies from vaccinated rata inhibited Tau uptake. The data show that B103 cells treated with pHrodo-PFF Tau exhibited significant Tau reuptake, and that Bepranemab (Bep), Semorinemab (Sem) or antibodies from rats vaccinated with p5555kb, p5187kb, or VXX-301 could comparably inhibit Tau reuptake. These results support the efficacy of the combination vaccine VXX-301 in inhibiting Tau reuptake and aggregation.

To evaluate in vivo protection against Tau-associated pathology and death, female P301L Tau mutant mice were vaccinated with saline control, p5555kb, p6187kb or VXX-301 for a total of 6 injections and monitored for 210 days. Vaccination with p5555kb, p6187kb or VXX-301 delayed the onset of ataxia (FIG. 12A) and prolonged survival (FIG. 12B) in a larger percentage of mice. Vaccination with p5555kb, p6187kb or VXX-301 also resulted in high titers of antibodies against both monomeric (FIG. 12C) and fibril PFF (FIG. 12D) Tau in P301L mice.

### Example 4

The epitope specificity of anti-Tau antibodies generated with p5555kb, p5187kb, or VXX-301 was determined by epitope mapping with ELISA. ELISA assay plates were coated with 2 mg/mL of each of the target peptide in Table B, immobilized to the plate through a biotin linker, in 1×TBS and incubated overnight at 2-8° C. Target peptides “N Terminal Tau (1-22)” and “p5555kb Targeting Sequence” resemble the epitope used in generating p5555kb-induced antibodies. Target peptides “p5187kb Targeting Sequence”, “P301S p5187kb”, “P301L p5187kb”, and “3R Tau p5187kb” resemble the epitope used in generating p5187kb-induced antibodies. Coated plates were blocked in PBS SuperBlock™ Buffer (SBB; Thermo Fisher) for 2 h at 28° C. with shaking at 500 rpm. Serum samples from p5555kb, p5187kb, or VXX-301 vaccinated animals were serially diluted in half-log iterations from an initial factor of 20 in 1×PBS containing 2% BSA across a 12-point series, then arrayed in duplicate into assay plates and incubated for 1 h at 28° C. HRP-conjugated anti-rat IgG was used for titer detection at a 1:10,000 dilution in SBB. After incubation at 28° C., 3.3′,5,5′-tetramethylbenzidine (TMB) Ultra ELISA substrate was applied to each plate and the reaction was stopped after 20 minutes by addition of 2N H₂SO₄ acid. Absorbance was measured at 450 nm on a SpectraMax iD5 plate reader (Molecular Devices) within 10 minutes of halting the reaction. A non-linear four-parameter (4-PL) curve fit was applied to each dilution series to calculate the EC₅₀ titers (SoftMax Pro, ver. 7.1 or GraphPad Prism, ver. 9.2).

TABLE B
EC₅₀ Titers of Epitope Mapping

| | Description of | | | |
|---|---|---|---|---|
| | the target | | | |
| Target peptide | peptide | p5555kb | p5187kb | VXX-301 |
| NH2- | Tau (1-22) | 177035 | 27.92 | 31675 |
| MAEPRQEFEVMEDHAGTYGLGD | N Terminal | | | |
| Ac-DHAGTYGLGD | p5555kb | 4539 | 69.06 | 3525 |
| | Targeting | | | |
| | Sequence | | | |
| IKHVPGGGSVQIVYK | p5187kb | 118.5 | 58060 | 27355 |
| | Targeting | | | |
| | Sequence | | | |
| IKHVSGGGSVQIVYK | P301S p5187kb | 182.1 | 14660 | 12323 |
| IKHVLGGGSVQIVYK | P301L | 193.9 | 14989 | 8956 |
| | p5187kb | | | |
| LKHQPGGGKVQIINK | 3R Tau | 47.46 | 6297 | 4727 |
| | p5187kb | | | |

Ec₅₀ values generated via 12-point dilution curve ELISAs probing against the target peptides using the terminal bleed sera (15 weeks) from vaccinated male Hartley guinea pigs.

As shown in Table B, p5555kb-induced anti-Tau antibodies showed robust EC₅₀ titers and binding potency to target peptides resembling p5555kb B-cell epitope, while p5187kb-induced anti-Tau antibodies showed robust EC₅₀ titers and binding potency to target peptides resembling p5187kb B-cell epitope. VXX-301 generated antibodies showed high EC₅₀ titers and binding potency for all target peptides used. These results support that p5555kb- and p5187kb-induced antibodies bind different target epitopes, and the efficacy of the combination vaccine VXX-301 against Tau.

### Example 5

A combination vaccine was designed by combining a Aβ targeting peptide vaccine, and a Tau targeting peptide vaccine in one vaccine formulation. Immunogenicity of vaccines were evaluated in guinea pigs, rats, and mice. Vaccine derived antibodies were characterized by Western Blot, dot blot, and immunohistochemistry. Vaccine efficacy was assessed in double transgenic mice (P301L) expressing both human Aβ and Tau, with a focus on Aβ and Tau brain pathology by immunohistochemistry and various immunoassays in brain homogenates.

FIGS. 13A and 13B show Aβ-Tau combination vaccine antibody titers against Tau and Aβ. Three different Tau peptide immunogen constructs were used (p5555kb, p5404kb, and p5137kb) in the combinations with Aβ peptide immunogen construct (p3102kb). p3102kb is the construct of SEQ ID NO: 70 shown in Table 4. The results in FIGS. 13A-13B show that combination vaccines induced robust immunogenicity with high titers against both oligomeric Aβ and a range of Tau forms (monomeric, oligomeric, and fibrils).

TABLE 1
Amino Acid Sequences of Tau B cell epitopes

| | Tau | | |
|---|---|---|---|
| | sequence | Peptide | SEQ |
| Sequences | region | Code | ID NO |
| MAEPRQEFEVMEDHAGTYGLGD | 1-22 | | 1 |
| DHAGTYGLGD | 13-22 | | 2 |
| NITHVPGGGNKK | 359-370 | | 3 |
| DNIKHVPGGGSVQIVYK | 295-311 | | 4 |
| TKIATPRGAAPP | 149-160 | | 5 |
| VVRTPPKSPSSAKSRL | 228-243 | | 6 |
| IKHVPGGGSVQIVYK | 297-311 | | 7 |
| KHVPGGGSV | 298-306 | | 202 |
| VSGDTSPRHLSNVSST | 399-414 | | 8 |
| AcIKHVPGGGSVQIVYK | 297-311 | | 72 |
| I(NMe)KHVPGGGSVQIVYK | 297-311 | | 73 |
| (NMe)IKHVPGGGSVQIVYK | 297-311 | | 74 |
| (NMe)I(NMe)KHVPGGGSVQIVYK | 297-311 | | 75 |
| IKHCPGCGSVQIVYK* | 297-311 | | 76 |
| LKHQPGGGKVQIVYK | | | 96 |
| MAEPRQEFEV | | p5543a | 17 |
| PROEFEVMED | | p5546a | 18 |
| RQEFEVMEDH | | p5547a | 19 |
| DHAGTYGLGD | | p5555a | 20 |
| AGTYGLGDRK | | p5557a | 21 |
| GTYGLGDRKD | | p5558a | 22 |
| TYGLGDRKDQ | | p5559a | 23 |
| DRKDQGGYTM | | p5564a | 24 |
| RKDQGGYTMH | | p5565a | 25 |
| KDQGGYTMHQ | | p5566a | 26 |
| GGYTMHQDQE | | P5569a | 27 |
| KIATPRGAÅP | | p5695a | 28 |
| IATPRGAAPP | | p5696a | 29 |
| VSGDTSPRHL | | p5535a | 30 |
| HAGTYGLGDR | | p5556a | 63 |
| MAEPRQEFEVMEDHAGTYGLGDRKDQGGYTMHQDQEGDTDAGLKE | | p5456a | 64 |
| ADGKTKIATPRGAAPP | | p4579a | 65 |
| PRHLSNVSST | | p5541a | 66 |
| RENAKAKTDHGAEIVYKSPVVSGDTSPRHL | | p4062a | 67 |

Ac stands for acetylated. In some embodiments of any of the B cell epitopes presented in Table 1, the acetylation, where present, can be removed. In some embodiments of any of the B cell epitopes presented in Table 1, the acetylation, where present, can be replaced with another modification protecting the N-terminal end from protease degradation. For example, such modifications on the N-terminus include, without limitation, adding glycines (e.g., GGG), propionylation, adding benzyloxycarbonyl group, adding a short PEG, or adding a D-amino acid. In other embodiments, where absent, acetylation can be added at the N-terminal end.
N-Me stands for N-methylated (mono methylated amino acid, attached to the following amino acid by an amide bond).
*This peptide is cyclized by cysteine disulfide bond (see cysteines in bold).

TABLE 2
Amino Acid Sequences of Tau Peptide Immunogen Constructs

| | | | Target | SEQ |
|---|---|---|---|---|
| | | | sequence | ID |
| | Designation | Sequences | region | NO |
| | p5459kb | MAEPRQEFEVMEDHAGTYGLGD-KKK-εK-ISITEIKGVIVHRIETILF- | 1-22 | 9 |
| | | NH2 | | |
| A | p5555kb | DHAGTYGLGD-KKK-εK-ISITEIKGVIVHRIETILF-NH2 | 13-22 | 10 |
| B | p5404kb | ISITEIKGVIVHRIETILF-εK-KKK-NITHVPGGGNKK-NH2 | 359-370 | 11 |
| C | p5137kb | ISITEIKGVIVHRIETILF-εK-KKKK-DNIKHVPGGGSVQIVYK-NH2 | 295-311 | 12 |
| D | p5505kb | ISITEIKGVIVHRIETILF-εK-KKK-TKIATPRGAAPP-NH2 | 149-160 | 13 |
| E | p4598kb | ISITEIKGVIVHRIETILF-εK-KKK-VVRTPPKSPSSAKSRL-NH2 | 228-243 | 14 |
| F | p5187kb | ISITEIKGVIVHRIETILF-εK-KKK-IKHVPGGGSVQIVYK-NH2 | 297-311 | 15 |
| G | p5506kb | ISITEIKGVIVHRIETILF-εK-KKK-VSGDTSPRHLSNVSST-NH2 | 399-414 | 16 |
| H | Ac- | Ac-IKHVPGGGSVQIVYK-KKKεK-ISITEIKGVIVHRIETILF | 297-311 | 77 |
| | p5187kb | | | |
| I | NMe-K- | Ac-ISITEIKGVIVHRIETILF-εKKKK-I-(NMe)KHVPGGGSVQIVYK | 297-311 | 78 |
| | p5187kb | | | |
| J | nMe-I- | Ac-ISITEIKGVIVHRIETILF-εKKKK-(NMe)IKHVPGGGSVQIVYK | 297-311 | 79 |
| | p5187kb | | | |
| K | NMe-IK- | Ac-ISITEIKGVIVHRIETILF-εKKKK- | 297-311 | 80 |
| | p5187kb | (NMe)I(NMe)KHVPGGGSVQIVYK | | |
| L | Cys-Cys- | | 297-311 | 81 |
| | p5187kb | Ac-IKHCPGCGSVQIVYK-KKKεK-ISITEIKGVIVHRIETILF | | |

The target peptide sequences are underlined.
Ac stands for acetylated. In some embodiments of any of the peptide immunogen constructs presented in Table 2, the acetylation, where present, can be removed. In some embodiments of any of the peptide immunogen constructs presented in Table 2, the acetylation, where present, can be replaced with another modification protecting the N-terminal end from protease degradation. For example, such modifications on the N-terminus include, without limitation, adding glycines (e.g., GGG), propionylation, adding benzyloxycarbonyl group, adding a short PEG, or adding a D-amino acid. In other embodiments, where absent, acetylation can be added at the N-terminal end.
N-Me stands for N-methylated (mono methylated amino acid, attached to the following amino acid by an amide bond).

TABLE 3
Amino Acid Sequences of Aβ molecules and B cell epitopes

| | SEQ ID | |
|---|---|---|
| | NO | Sequences |
| Aβ₁₋₄₂ | 68 | DAEFRHDSGYEVHHQKLVFFAEDVGSNKGAIIGLMVGGVVIA |
| Aβ₁₋₁₄ | 69 | DAEFRHDSGYEVHH |
| pyroE-Aβ | 82 | pEFRHDSGYEVHH |
| Aβ₁₋₁₀ | 83 | DAEFRHDSGY |
| Aβ₁₋₁₂ | 84 | DAEFRHDSGYEV |
| Aβ₁₋₂₈ | 85 | DAEFRHDSGYEVHHQKLVFFAEDVGSNK |
| Aβ₅₋₁₄ | 86 | RHDSGYEVHH |
| Aβ₆₋₁₅ | 87 | HDSGYEVHHQ |
| Aβ₇₋₁₆ | 88 | DSGYEVHHQK |

pE is a pyroglutamic acid.

TABLE 4
Amino Acid Sequences of Aβ Peptide Immunogen Constructs

| | SEQ ID | |
|---|---|---|
| | NO | Sequences |
| p3102kb | 70 | DAEFRHDSGYEVHH-KKKεK-ISITEIKGVIVHRIETILF |
| | 71 | DAEFRHDSGYEVHH-KKKεK-IITITRIITIITTID |
| pyroE-Aβ | 89 | pEFRHDSGYEVHH-KKKεK-ISITEIKGVIVHRIETILF |
| (pyroglutamine | | |
| Aβ) | | |

The target peptide sequences are underlined.
pE is a pyroglutamic acid.

TABLE 5
Amino Acid Sequences of Pathogen Protein Derived Th Epitopes Including
Idealized Artificial Th Epitopes for Employment in the Design of Tau
Peptide Immunogen Constructs

| | | SEQ ID |
|---|---|---|
| Description | Sequence* | NO |
| Clostridium tetani1 Th | KKQYIKANSKFIGITEL | 31 |
| MvF1 Th | LSEIKGVIVHRLEGV | 32 |
| Bordetella pertussis Th | GAYARCPNGTRALTVAELRGNAEL | 33 |
| Clostridium tetani2 Th | WVRDIIDDFTNESSQKT | 34 |
| Diphtheria Th | DSETADNLEKTVAALSILPGHGC | 35 |
| Plasmodium falciparum Th | DHEKKHAKMEKASSVFNVVNS | 36 |
| Schistosoma mansoni Th | KWFKTNAPNGVDEKHRH | 37 |
| Cholera Toxin Th | ALNIWDRFDVFCTLGATTGYLKGNS | 38 |
| MvF2 Th | ISEIKGVIVHKIEGI | 39 |
| KKKMvF3 Th | KKKISISEIKGVIVHKIEGILF | 40 |
| | T RT TR T | |
| HBsAg1 Th | KKKLFLLTKLLTLPQSLD | 41 |
| | RRRIKII RII I L IR | |
| | VRVV VV V I V | |
| | F FF FF F V F | |
| MvF4 Th (UBITh ®3) | ISISEIKGVIVHKIETILF | 42 |
| | T RT TR | |
| HBsAg2 Th | KKKIITITRIITIPQSLD | 43 |
| | FFLL T ITTI | |
| MvF5 Th (UBITh ®1) | ISITEIKGVIVHRIETILF | 44 |
| HBsAg3 Th (UBITh ®2) | KKKIITITRIITIITTID | 45 |
| Influenza MP1_1 Th | FVFTLTVPSER | 46 |
| Influenza MP1_2 Th | SGPLKAEIAQRLEDV | 47 |
| Influenza NSP1 Th | DRLRRDQKS | 48 |
| EBV BHRF1 Th | AGLTLSLLVICSYLFISRG | 49 |
| Clostridium tetani TT1 Th | QYIKANSKFIGITEL | 50 |
| EBV EBNA-1 Th | PGPLRESIVCYFMVFLQTHI | 51 |
| Clostridium tetani TT2 Th | FNNFTVSFWDRVPKVSASHLE | 52 |
| Clostridium tetani TT3 Th | KFIIKRYTPNNEIDSF | 53 |
| Clostridium tetani TT4 Th | VSIDKFRIFCKALNPK | 54 |
| EBV CP Th | VPGLYSPCRAFFNKEELL | 55 |
| HCMV IE1 Th | DKREMWMACIKELH | 56 |
| EBV GP340 Th | TGHGARTSTEPTTDY | 57 |
| EBV BPLF1 Th | KELKRQYEKKLRQ | 58 |
| EBV EBNA-2 Th | TVFYNIPPMPL | 59 |
| MvF4 Th (UBITh ®3) | ISISEIKGVIVAKIETILF | 97 |

*For the Th epitopes comprising “KKK” at the N-terminus, the “KKK” can be removed for use in the peptide immunogens described herein, in particular, those comprising a spacer comprising “KKK” sequence.

TABLE 6
Tau protein sequences

| Description | Sequence and SEQ ID NO | |
|---|---|---|
| Tau protein | MAEPRQEFEVMEDHAGTYGLGDRKDQGGYTMHQDQEGDTDAGLKESPLQTPTEDGSEEPGSETSDAKSTP | 70 |
| sequence, | TAEDVTAPLVDEGAPGKQAAAQPHTEIPEGTTAEEAGIGDTPSLEDEAAGHVTQARMVSKSKDGTGSDDK | 140 |
| full length | KAKGADGKTKIATPRGAAPPGQKGQANATRIPAKTPPAPKTPPSSGEPPKSGDRSGYSSPGSPGTPGSRS | 210 |
| (441 aa) | RTPSLPTPPTREPKKVAVVRTPPKSPSSAKSRLQTAPVPMPDLKNVKSKIGSTENLKHQPGGGKVQIINK | 280 |
| isoform with | KLDLSNVQSKCGSKDNIKHVPGGGSVQIVYKPVDLSKVTSKCGSLGNIHHKPGGGQVEVKSEKLDFKDRV | 350 |
| 4R and 2N | QSKIGSLDNITHVPGGGNKKIETHKLTFRENAKAKTDHGAEIVYKSPVVSGDTSPRHLSNVSSTGSIDMV | 420 |

| | |
|---|---|
| GenBank: | DSPQLATLADEVSASLAKQGL |
| AGF19246.1 | (SEQ ID NO. 60) |
| Tau isoform | MAEPRQEFEVMEDHAGTYGLGDRKDQGGYTMHQDQEGDTDAGLKAEEAGIGDTPSLEDEAAGHVTQARMVSKSKDGTG |
| 0N3R | SDDKKAKGADGKTKIATPRGAAPPGQKGQANATRIPAKTPPAPKTPPSSGEPPKSGDRSGYSSPGSPGTPGSRSRTPSL |
| | PTPPTREPKKVAVVRTPPKSPSSAKSRLQTAPVPMPDLKNVKSKIGSTENLKHQPGGGKVQIVYKPVDLSKVTSKCGSL |
| | GNIHHKPGGGQVEVKSEKLDFKDRVQSKIGSLDNITHVPGGGNKKIETHKLTFRENAKAKTDHGAEIVYKSPVVSGDTS |
| | PRHLSNVSSTGSIDMVDSPQLATLADEVSASLAKQGL |
| | (SEQ ID NO. 90) |
| Tau isoform | MAEPRQEFEVMEDHAGTYGLGDRKDQGGYTMHQDQEGDTDAGLKESPLQTPTEDGSEEPGSETSDAKSTPTAEAEEAGI |
| 1N3R | GDTPSLEDEAAGHVTQARMVSKSKDGTGSDDKKAKGADGKTKIATPRGAAPPGQKGQANATRIPAKTPPAPKTPPSSGE |
| | PPKSGDRSGYSSPGSPGTPGSRSRTPSLPTPPTREPKKVAVVRTPPKSPSSAKSRLQTAPVPMPDLKNVKSKIGSTENL |
| | KHQPGGGKVQIVYKPVDLSKVTSKOGSLGNIHHKPGGGQVEVKSEKLDFKDRVQSKIGSLDNITHVPGGGNKKIETHKL |
| | TFRENAKAKTDHGAEIVYKSPVVSGDTSPRHLSNVSSTGSIDMVDSPQLATLADEVSASLAKQGL |
| | (SEQ ID NO. 91) |
| Tau isoform | MAEPRQEFEVMEDHAGTYGLGDRKDQGGYTMHQDQEGDTDAGLKESPLQTPTEDGSEEPGSETSDAKSTPTAEDVTAPL |
| 2N3R | VDEGAPGKQAAAQPHTEIPEGTTAEEAGIGDTPSLEDEAAGHVTQARMVSKSKDGTGSDDKKAKGADGKTKIATPRGAA |
| | PPGQKGQANATRIPAKTPPAPKTPPSSGEPPKSGDRSGYSSPGSPGTPGSRSRTPSLPTPPTREPKKVAVVRTPPKSPS |
| | SAKSRLQTAPVPMPDLKNVKSKIGSTENLKHQPGGGKVQIVYKPVDLSKVTSKCGSLGNIHHKPGGGQVEVKSEKLDFK |
| | DRVQSKIGSLDNITHVPGGGNKKIETHKLTFRENAKAKTDHGAEIVYKSPVVSGDTSPRHLSNVSSTGSIDMVDSPQLA |
| | TLADEVSASLAKQGL (SEQ ID NO. 92) |
| Tau isoform | MAEPRQEFEVMEDHAGTYGLGDRKDQGGYTMHQDQEGDTDAGLKAEEAGIGDTPSLEDEAAGHVTQARMVSKSKDGTG |
| ON4R | SDDKKAKGADGKTKIATPRGAAPPGQKGQANATRIPAKTPPAPKTPPSSGEPPKSGDRSGYSSPGSPGTPGSRSRTPSL |
| | PTPPTREPKKVAVVRTPPKSPSSAKSRLQTAPVPMPDLKNVKSKIGSTENLKHQPGGGKVQIINKKLDLSNVQSKCGSK |
| | DNIKHVPGGGSVQIVYKPVDLSKVTSKCGSLGNIHHKPGGGQVEVKSEKLDFKDRVQSKIGSLDNITHVPGGGNKKIET |
| | HKLTFRENAKAKTDHGAEIVYKSPVVSGDTSPRHLSNVSSTGSIDNIVDSPQLATLADEVSASLAKQGL |
| | (SEQ ID NO. 93) |
| Tau isoform | MAEPRQEFEVMEDHAGTYGLGDRKDQGGYTMHQDQEGDTDAGLKESPLQTPTEDGSEEPGSETSDAKSTPTAEAEEAGI |
| 1N4R | GDTPSLEDEAAGHVTQARMVSKSKDGTGSDDKKAKGADGKTKIATPRGAAPPGQKGQANATRIPAKTPPAPKTPPSSGE |
| | PPKSGDRSGYSSPGSPGTPGSRSRTPSLPTPPTREPKKVAVVRTPPKSPSSAKSRLQTAPVPMPDLKNVKSKIGSTENL |
| | KHQPGGGKVQIINKKLDLSNVQSKCGSKDNIKHVPGGGSVQIVYKPVDLSKVTSKCGSLGNIHHKPGGGQVEVKSEKLD |
| | FKDRVQSKIGSLDNITHVPGGGNKKIETHKLTFRENAKAKTDHGAEIVYKSPVVSGDTSPRHLSNVSSTGSIDMIVDSP |
| | QLATLADEVSASLAKQGL (SEQ ID NO, 94) |

TABLE 7
Localization of immunogenic regions within Tau 275-311 through fine epitope mapping
(Table 7 discloses SEQ ID NOS 98-105, 4, 7, 106-113, respectively, in order of appearance)
Tau (275-311) B epitope mapping for p4604kb

| | | Hyperimmune serum PTAU-15-01GP |
|---|---|---|
| | | grp 10 (4603kb, UBITh1-εk-kkk- |
| 275 311 | | (V275-K311, P301→S301), 6w |
| VQIINKKLDLSNVQSKCGSKDNIKHVPGGGSVQIVYK | | Abs (450nm) |
| p5116kb: KLDLSNVQSKCGSKDNIKHVPGGGSVQIVYK | (281-311) | 3.782 |
| p5117kb: DLSNVQSKCGSKDNIKHVPGGGSVQIVYK | (283-311) | 3.802 |
| p5118kb: SNVQSKCGSKDNIKHVPGGGSVQIVYK | (285-311) | 3.788 |
| p5119kb: VQSKCGSKDNIKHVPGGGSVQIVYK | (287-311) | 3.836 |
| p5120kb: SKCGSKDNIKHVPGGGSVQIVYK | (289-311) | 3.826 |
| p5121kb: CGSKDNIKHVPGGGSVQIVYK | (291-311) | 3.781 |
| p5122kb: SKDNIKHVPGGGSVQIVYK | (293-311) | 3.837 |
| p5123kb: DNIKHVPGGGSVQIVYK | (295-311) | 3.732 |
| p5124kb: IKHVPGGGSVQIVYK | (297-311) | 3.477 |
| p5125kb: HVPGGGSVQIVYK | (299-311) | 0.221 |
| p5126kb: PGGGSVQIVYK | (301-311) | 0.099 |
| p5127kb: VQIINKKLDLSNVQSKCGSKDNIKHVPGGGS | (275-305) | 0.148 |
| p5128kb: VQIINKKLDLSNVQSKCGSKDNIKHVP | (275-301) | 0.283 |
| p5129kb: VQIINKKLDLSNVQSKCGSKDN | (275-297) | 0.303 |
| p5130kb: VQIINKKLDLSNVQSKCGS | (275-293) | 0.320 |
| p5131kb: VQIINKKLDLSNVQS | (275-289) | 0.091 |
| p5132kb: VQIINKKLDLS | (275-285) | 0.095 |
| 4604a (V275-K311) | (275-311) | 3.994 |

Hyperimmune serums from:
Pooled group 10 (4603kb, UBITh1-εk-kkk-(V275-K311, P301→S301) of PTAU-15-01GP, 6wpi.
Positive peptide: 4604a (V275-K311)
Hyperimmune serum 1/10000 dilution for mapping peptide
Hyperimmune serum 1/10000 dilution for positive peptide

TABLE 8
Localization of immunogenic regions within Tau 1-45 through fine epitope mapping
(Table 8 discloses 64, 17, 114-115, 18, 19, 116-122, 2, 63,
21-23, 123-126, 24-26, 127, 128, 27, 129-137 and
64, respectively, in order of appearance)
Tau N terminal 1-45 10 mer epitope mapping

| | | | Antiserums from Tau-18-05GP study | | | |
|---|---|---|---|---|---|---|
| | | | 1. | 2. | 3. | 4. |
| | | | p5456kb: | p5457kb: | p5458kb: | p5459kb: |
| | | | Tau | Tau | Tau | Tau |
| | | | (M1-E45)- | (M1-D40)- | (M1-D34)- | ( M1-D22)- |
| | | | KKK; ϵK- | KKK-ϵK- | KKK-ϵK- | KKK-ϵK- |
| | | | UBITh1, | UBITh1, | UBITh1, | UBITh1, |
| | | | grp 6, | grp 7, | grp 8, | grp 9, |
| | | Pep- | 15wpi | 15wpi | 15wpi | 15wpi |
| MAEPRQEFEVMEDHAGTYGLGDRKDQGGYTMHQDQEGDTDAGLKE | | tide | Abs450 (Serum samples at | | | |
| 1 45 | Region | code | 1:100 dilution) | | | |
| MAEPRQEFEV | 1-10 | p55433 | 1.149 | 0.476 | 3.027 | 2.826 |
| AEPRQEFEVM | 2-11 | p5544a | 0.210 | 0.084 | 0.666 | 0.224 |
| EPRQEFEVME | 3-12 | p5545a | 0.191 | 0.170 | 0.345 | 0.288 |
| PRQEFEVMED | 4-13 | p5546a | 2.254 | 2.554 | 2.352 | 3.349 |
| RQEFEVMEDH | 5-14 | p55478 | 1.669 | 1.889 | 1.253 | 2.111 |
| QEFEVMEDHA | 6-15 | p5548a | 0.704 | 0.676 | 0.601 | 0.912 |
| EFEVMEDHAG | 7-16 | ps5493 | 0.161 | 0.145 | 0.209 | 0.237 |
| FEVMEDHAGT | 8-17 | p55508 | 0.076 | 0.082 | 0.088 | 0.118 |
| EVMEDHAGTY | 9-18 | p5551a | 0.078 | 0.097 | 0.166 | 0.088 |
| VMEDHAGTYG | 10-19 | p5552a | 0.093 | 0.095 | 0.486 | 0.114 |
| MEDHAGTYGL | 11-20 | p5553a | 0.154 | 0.107 | 0.334 | 0.386 |
| EDHAGTYGLG | 12-21 | p5554a | 0.080 | 0.068 | 0.098 | 0.069 |
| DHAGTYGLGD | 13-22 | p55558 | 1.449 | 0.309 | 3.322 | 1.765 |
| HAGTYGLGQR | 14-23 | p5556a | 0.853 | 0.737 | 0.525 | 2.395 |
| AGTYGLGDRK | 15-24 | p5557a | 0.490 | 1.630 | 0.891 | 3.537 |
| GTYGLGDRKD | 16-25 | p5558a | 2.178 | 3.334 | 2.255 | 3.946 |
| TYGLGDRKDQ | 17-26 | p5559a | 3.011 | 3.017 | 2.064 | 3.658 |
| YGLGDRKDQG | 18-27 | p5560a | 0.371 | 0.286 | 0.204 | 0.456 |
| GLGDRKDQGG | 19-28 | p55618 | 0.091 | 0.064 | 0.093 | 0.088 |
| LGDRKDQGGY | 20-29 | p5562a | 0.078 | 0.092 | 0.072 | 0.153 |
| GDRKDQGGYT | 21-30 | p5563a | 0.221 | 0.493 | 0.533 | 0.527 |
| DRKDQGGYTM | 22-31 | p5564a | 0.768 | 1.872 | 2.140 | 0.511 |
| RKDQGGYTMH | 23-32 | p5565a | 0.93 | 1.712 | 2.262 | 0.318 |
| KDQGGYTMHQ | 24-33 | p55668 | 0.961 | 0.911 | 1.183 | 0.386 |
| DQGGYTMHQD | 25-34 | p5567a | 0.140 | 0.127 | 0.094 | 0.093 |
| QGGYTMHQDQ | 26-35 | p5568a | 0.398 | 0.194 | 0.924 | 0.413 |
| GGYIMHQDQE | 27-36 | p55698 | 0.272 | 0.268 | 1.011 | 0.650 |
| GYTMHQDQEG | 28-37 | p5570a | 0.275 | 0.098 | 0.614 | 0.688 |
| YTMHQDQEGD | 29-38 | p5571a | 0.101 | 0.128 | 0.114 | 0.213 |
| TMHQDQEGDT | 30-39 | p5572a | 0.072 | 0.065 | 0.068 | 0.078 |
| MHQDQEGDTD | 31-40 | p5573a | 0.064 | 0.061 | 0.068 | 0:063 |
| HQDQEGDTDA | 32-41 | p5574a | 0.062 | 0.052 | 0.055 | 0.053 |
| QDQEGDIDAG | 33-42 | p5575a | 0.065 | 0.059 | 0.057 | 0.061 |
| DQEGDTDAGL | 34-43 | p5576a | 0.080 | 0.066 | 0.062 | 0.061 |
| QEGDIDAGLK | 35-44 | p5577a | 0.091 | 0.061 | 0.067 | 0.075 |
| EGDIDAGLKE | 36-45 | p5578a | 0.079 | 0.079 | 0.088 | 0.077 |
| MAEPRQEFEVMEDHAGTYGLGDRKDQGGYTMHQDQEGDTDAGLKE | 1-45 | p5456a | 4.000 | 4.000 | 4.000 | 4.000 |

Positive control peptides: p5456a, Tau ‘a’ peptide M1-E45)
Hyperimmune serums were tested on the positive control peptides with 1/10000 dilution.

TABLE 9
Localization of immunogenic regions within Tau 145-160 through fine epitope mapping (Table 9
discloses SEQ ID NOS 138-152, 28, 29, 153-162 and 65, respectively, in order of appearance)
Tau 10 mer peptides for epitope mapping within 145-160 region

| | | | Antiserums | | | |
|---|---|---|---|---|---|---|
| | | | 1. | 2. | 3. | 4. |
| | | | p4579kb: | p4579kb: | p5200kb: | p5505kb, |
| | | | UBITh1- | UBITh1- | UBITh3- | UBITh1- |
| | | | ϵK-kkk- | ϵK-kkk- | (A145- | ϵK-kkk- |
| | | | (A145- | (A145- | P160), | (T149- |
| | | | P160), | P160), | grp 2 | P160) |
| | | | Grp 1 | Grp 1 | of | grp 4 |
| | | | of | of | Tau-18- | of |
| | | | Tau- | Tau- | 04GP | Tau- |
| | | | 15-02GP, | 19-01GP, | V2, | 19-02GP, |
| GSDDKKAKGADGKTKIATPRGAAPPGQKGQANATR | | Peptide | 9w | 9w | 6w | 3w |
| 136 170 | Regions | code | Abs450 (Serum samples at 1:100 dilution) | | | |
| GSDDKKAKGA | 136-145 | p5681a | 0.080 | 0.067 | 0.072 | 0.066 |
| SDDKKAKGAD | 137-146 | p5682a | 0.071 | 0.061 | 0.075 | 0.065 |
| DDKKAKGADG | 138-147 | p5683a | 0.081 | 0.078 | 0.071 | 0.061 |
| DKKAKGADGK | 139-148 | p5684a | 0.073 | 0.058 | 0.066 | 0.067 |
| KKAKGADGKT | 140-149 | p5685a | 0.075 | 0.058 | 0.062 | 0.064 |
| KAKGADGKTK | 141-150 | p5686a | 0.076 | 0.063 | 0.073 | 0.067 |
| AKGADGKTKI | 142-151 | p5687a | 0.068 | 0.060 | 0.071 | 0.067 |
| KGADGKTKIA | 143-152 | p5688a | 0.072 | 0.060 | 0.069 | 0.075 |
| GADGKIKIAT | 144-153 | p5689a | 0.086 | 0.067 | 0.071 | 0.068 |
| ADGKTKIATP | 145-154 | p5690a | 0.788 | 0.073 | 0.076 | 0.066 |
| DGKTKIATPR | 146-155 | p5691a | 0.092 | 0.06€ | 0.074 | 0.064 |
| GKTKIATPRG | 147-156 | p5692a | 0.181 | 0.082 | 0.085 | 0.076 |
| KTKIATPRGA | 148-157 | p5693a | 0.113 | 0.098 | 0.082 | 0.082 |
| TKIATPRGAA | 149-158 | p5694a | 0.107 | 0.054 | 0.060 | 0.060 |
| KIATPRGAAP | 150-159 | p5695a | 3.729 | 3.435 | 0.125 | 0.125 |
| IATPRGAAPP | 151-160 | p5696a | 3.918 | 3.915 | 3.974 | 2.721 |
| ATPRGAAPPG | 152-161 | p5697a | 0.430 | 0.246 | 0.173 | 0.259 |
| TPRGAAPPGQ | 153-162 | p5698a | 0.077 | 0.049 | 0.063 | 0.051 |
| PRGAAPPGQK | 154-163 | p5699a | 0.081 | 0.050 | 0.059 | 0.066 |
| RGAAPPGQKG | 155-164 | p5700a | 0.071 | 0.045 | 0.055 | 0.057 |
| GAAPPGQKGQ | 156-165 | p5701a | 0.086 | 0.054 | 0.057 | 0.063 |
| AAPPGQKGQA | 157-166 | p5702a | 0.076 | 0.058 | 0.065 | 0.064 |
| APPGQKGQAN | 158-167 | p5703a | 0.087 | 0.060 | 0.065 | 0.070 |
| PPGQKGQANA | 159-168 | p5704a | 0.071 | 0.053 | 0.055 | 0.057 |
| PGQKGQANAT | 160-169 | p5705a | 0.101 | 0.075 | 0.074 | 0.067 |
| GQKGQANATR | 161-170 | p5706a | 0.090 | 0.070 | 0.074 | 0.066 |
| ADGKTKIATPRGAAPP | 145-160 | p4579a | 3.686 | 3.254 | 3.854 | 0.977 |

Positive control peptides: p4579a, Tau ‘a’ peptide A145-P160)
Hyperimmune serums were tested on the positive control peptides with 1/10000 dilution.

TABLE 10
Localization of immunogenic regions within Tau 379-408 through fine epitope mapping
(Table 10 discloses SEQ ID NOS 163-191, 30, 192-196, 66, 197 and 198, respectively,
Tau 10 mer peptides for epitope mapping within 379-408 region

| | | | 1. | 2. |
|---|---|---|---|---|
| | | | p4602kb: | p4602kb: |
| | | | UBITh1 | UBITh1 |
| | | | ϵK-kkk- | ϵK-kkk- |
| | | | (R379-L408), | (R379-1408), |
| | | | Tau-15- | Tau-19- |
| | | | 02GP, | 01GP, |
| | | | grp 8, | grp 2, |
| | | Pep- | 9wpi | 9wpi. |
| IETHKLTFRENAKAKTDHGAEIVYKSPVVSGDTSPRHLSNVSSTG | IAPP | tide | Abs450 | |
| 371 415 | region | code | (mean, n = 2) @ 1/100 dil | |
| IETHKLTFRE | 371-380 | p55078 | 0.088 | 0.083 |
| ETHKLTFREN | 372-381 | p5508a | 0.104 | 0.066 |
| THKLTFRENA | 373-382 | p55093 | 0.096 | 0.059 |
| HKLTFRENAK | 374-383 | p5510a | 0.096 | 0.067 |
| KLTERENAKA | 375-384 | p5511a | 0.089 | 0.063 |
| LTFRENAKAK | 376-385 | p5512a | 0.084 | 0.066 |
| TFRENAKAKT | 377-386 | p55133 | 0.094 | 0.057 |
| FRENAKAKTD | 378-387 | p5514a | 0.083 | 0.062 |
| RENAKAKTDH | 379-388 | p5515a | 0.089 | 0.061 |
| ENAKAKIDHG | 380-389 | 05516a | 0.097 | 0.065 |
| NAKAKTDHGA | 381-390 | p5517a | 0.130 | 0.104 |
| AKAKTDHGAE | 382-391 | 055188 | 0.135 | 0.080 |
| KAKTDHGAEI | 383-392 | p5519a | 0.638 | 0.210 |
| AKTDHGAEIV | 384-393 | p55208 | 0.109 | 0.070 |
| KTDHGAEIVY | 385-394 | p5521a | 0.286 | 0.201 |
| TDHGAEIVYK | 386-395 | p55223 | 0.305 | 0.251 |
| DHGAEIVYKS | 387-396 | 05523a | 0.119 | 0.104 |
| HGAEIVYKSP | 388-397 | p5524a | 0.157 | 0.151 |
| GAEIVYKSPV | 389-398 | p5525a | 0.183 | 0.147 |
| AEIVYKSPVV | 390-399 | p55263 | 0.159 | 0.152 |
| EIVYKSPVVS | 391-400 | 05527a | 0.106 | 0.082 |
| IVYKSPVVSG | 392-401 | p5528a | 0.120 | 0.077 |
| VYKSPVVSGD | 393-402 | 05529a | 0.107 | 0.070 |
| YKSPVVSGDT | 394-403 | p5530a | 0.111 | 0.072 |
| KSPVVSGDTS | 395-404 | 055318 | 0.100 | 0.061 |
| SPVVSGDTSP | 396-405 | p5532a | 0.100 | 0.069 |
| PVVSGDTSPR | 397-406 | p55333 | 0.106 | 0.171 |
| VVSGDTSPRH | 398-407 | p5534a | 0.113 | 0.076 |
| VSGDTSPRHL | 399-408 | p55353 | 4.000 | 4.000 |
| SGDTSPRHLS | 400-409 | p5536a | 0.186 | 0.529 |
| GDTSPRHLSN | 401-410 | p5537a | 0.123 | 0.170 |
| DTSPRHLSNV | 402-411 | p5538a | 0.105 | 0.085 |
| TSPRHLSNVS | 403-412 | p5539a | 0.108 | 0.086 |
| SPRHLSNVSS | 404-413 | 05540a | 0.106 | 0.083 |
| PRHLSNVSST | 405-414 | p5541a | 3.970 | 3.720 |
| RHLSNVSSTG | 406-415 | p5542a | 0.527 | 0.548 |
| RENAKAKTDHGAEIVYKSPVVSGDTSPRHL | 379-408 | p4602a | 4.000 | 4.000 |

Positve control peptide: p4062a (379-408). Samples are tasted on by 1/10000 dution.

TABLE 11
Exemplary CpG Oligonucleotides

| Des- | | SEQ ID |
|---|---|---|
| cription | Sequence/Composition* | NO |
| CpG1 | 5′ TCg TCg TTT TgT CgT TTT gTC gTT TTg TCg TT 3′ | 199 |
| CpG2 | Phosphate TCg TCg TTT TgT CgT TTT gTC gTT 3′ | 200 |
| CpG3 | 5′ TCg TCg TTT TgT CgT TTT gTC gTT 3′ | 201 |

*Nucleotides shown in capital letters signify phosphorothioate bonds and nucleotides shown in lowercase letters signify phosphodiester bonds,

What is claimed is:

1. A composition or a kit comprising a first Tau peptide immunogen construct comprising a first Tau B cell epitope comprising or consisting of 6 to about 40 amino acid residues from the full-length Tau protein of SEQ ID NO: 60; and
(i) a second Tau peptide immunogen construct comprising a second Tau B cell epitope comprising or consisting of 6 to about 40 amino acid residues from the full-length Tau protein of SEQ ID NO: 60, wherein the second B cell epitope is not the same as the first B cell epitope, and/or
(ii) an amyloid beta (Aβ) peptide immunogen construct comprising an amyloid beta B cell epitope comprising or consisting of 6 to about 42 amino acid residues from the full-length amyloid beta protein of SEQ ID NO: 68.

2. The composition or kit according to claim 1, which comprises the second Tau peptide immunogen construct.

3. The composition or kit according to claim 1, which comprises the amyloid beta peptide immunogen construct.

4. The composition or kit according to claim 1, which comprises the second Tau peptide immunogen construct and the amyloid beta peptide immunogen construct.

5. The composition or kit according to claim 1, 2 or 4, wherein the first Tau B cell epitope comprises or consists of 6 to about 40 amino acid residues from N-terminal amino acids 1 to 44 of SEQ ID NO: 60, and the second Tau B cell epitope comprises or consists of 6 to about 40 amino acid residues from repeat domain amino acids 244 to 372 of SEQ ID NO: 60.

6. The composition or kit according to claim 1, 3, 4 or 5, wherein the amyloid beta B cell epitope comprises at least 6 amino acid residues from amino acids 1-16 of SEQ ID NO: 68.

7. The composition or kit according to any one of claims 1-6, wherein the first Tau B cell epitope, the second Tau B cell epitope, and the amyloid beta B cell epitope are each about 8 to 15 amino acids in length, or 10 to 14 amino acids in length.

8. The composition or kit according to any one of claims 1-6, wherein the first Tau B cell epitope and, if present, the second Tau B cell epitope are independently selected from SEQ ID NOs: 1-8, 17-30, 63-67, 72-76, 96, and 202.

9. The composition or kit according to claim 8, wherein the first Tau B cell epitope and, if present, the second Tau B cell epitope are independently selected from SEQ ID NOs: 2, 4, 7, and 72-76.

10. The composition or kit according to any one of claims 1-6, wherein the first Tau B cell epitope comprises or consists of SEQ ID NO: 2.

11. The composition or kit according to any one of claims 1-6, wherein the first Tau B cell epitope comprises or consists of SEQ ID NO: 7.

12. The composition or kit according to any one of claims 1-6, wherein the first Tau B cell epitope is selected from SEQ ID No: 1 or SEQ ID NO:2, and the second Tau B cell epitope is selected from SEQ ID NOs: 7 and 72-76.

13. The composition or kit according to any one of claims 1-6, wherein the first Tau B cell epitope comprises or consists of SEQ ID NO: 2, and the second Tau B cell epitope comprises or consists of SEQ ID NO: 7.

14. The composition or kit according to any one of claims 1-13, wherein the amyloid beta B cell epitope is selected from SEQ ID NOs: 69 and 82-88.

15. The composition or kit according to any one of claims 1-13, wherein the amyloid beta B cell epitope comprises or consists of SEQ ID NO: 69 or SEQ ID NO: 82.

16. The composition or kit according to any one of claims 1-15, wherein the first Tau peptide immunogen construct comprises a first heterologous T helper (Th) epitope, the second Tau peptide immunogen construct, if present, comprises a second heterologous Th epitope, and the amyloid beta peptide immunogen construct, if present, comprises a third heterologous Th epitope.

17. The composition or kit according to claim 16, wherein the first heterologous Th epitope, the second heterologous Th epitope and the third heterologous Th epitope is each independently selected from SEQ ID NOs: 31-59 and 97.

18. The composition or kit according to claim 16, wherein the first heterologous Th epitope, the second heterologous Th epitope and the third heterologous Th epitope is each independently selected from SEQ ID NOs: 42, 44, 45 and 97.

19. The composition or kit according to claim 16, wherein each of the first heterologous Th epitope, the second heterologous Th epitope and the third heterologous Th epitope has SEQ ID NO: 44.

20. The composition or kit of any one of claims 1-19, wherein the first heterologous Th epitope and the first Tau B cell epitope, the second heterologous Th epitope and the second Tau B cell epitope, and the third heterologous Th epitope and the amyloid beta B cell epitope, are each covalently linked directly or through a heterologous spacer.

21. The composition or kit of claim 20, which comprises the heterologous spacer linking the first heterologous Th epitope and the first Tau B cell epitope, the second heterologous Th epitope and the second Tau B cell epitope, and/or the third heterologous Th epitope and amyloid beta B cell epitope.

22. The composition or kit of claim 21, wherein the heterologous spacer comprises one or more amino acids.

23. The composition or kit of claim 22, wherein the one or more amino acids is Lys-, Gly-, Lys-Lys-Lys-, (α, ε-N)Lys, Lys-Lys-Lys-ε-Lys (SEQ ID NO: 95), or ε-Lys-Lys-Lys-Lys (SEQ ID NO: 62).

24. The composition or kit of claim 22, wherein the one or more amino acids is Lys-Lys-Lys-ε-Lys (SEQ ID NO: 95) or ε-N-Lys-Lys-Lys-Lys (SEQ ID NO: 62).

25. The composition or kit according to any one of claims 1-24, wherein the first Tau peptide immunogen construct and, if present, the second Tau peptide immunogen construct, is represented by the formulae: 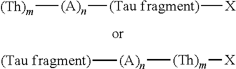
wherein
Th is a heterologous T helper epitope;
A is a heterologous spacer;
(Tau fragment) is a B cell epitope having 6 to about 40 amino acid residues from the full-length Tau protein of SEQ ID NO: 60; optionally wherein the B cell epitope has about 10 to about 40 amino acid residues from the full-length Tau protein of SEQ ID NO: 60;
X is an α-COOH or α-CONH₂ of an amino acid;
m is from 1 to about 4; and
n is from 0 to about 10.

26. The composition or kit according to any one of claims 1-25, wherein the amyloid beta peptide immunogen construct is represented by the formulae: 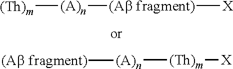
wherein
Th is a heterologous T helper epitope;
A is a heterologous spacer;
(Aβ fragment) is a B cell epitope having 6 to about 42 amino acid residues from the full-length Aβ protein of SEQ ID NO: 68; optionally wherein the B cell epitope has about 10 to about 42 amino acid residues from the full-length Aβ protein of SEQ ID NO: 68;
X is an α-COOH or α-CONH₂ of an amino acid;
m is from 1 to about 4; and
n is from 0 to about 10.

27. The composition or kit according to any one of claims 1-26, wherein the first Tau peptide immunogen construct comprises the first heterologous Th epitope covalently linked to the carboxyl terminus of the first Tau B cell epitope, the second Tau peptide immunogen construct comprises the second heterologous Th epitope covalently linked to the amino terminus of the second Tau B cell epitope, and/or the amyloid beta peptide immunogen construct comprises the third heterologous Th epitope covalently linked to the carboxyl terminus of the amyloid beta B cell epitope.

28. The composition or kit according to any one of claims 1-27, wherein the first Tau peptide immunogen construct and the second Tau peptide immunogen construct are selected from SEQ ID NOs: 9-16 and 77-81, or a construct in which the positions of the peptide cell epitope and the Th epitope are reversed.

29. The composition or kit according to any one of claims 1-28, wherein the first Tau peptide immunogen construct comprises or consists of the amino acid sequence of SEQ ID NO: 10 or SEQ ID NO: 15.

30. The composition or kit according to any one of claims 1-29, wherein the first Tau peptide immunogen construct comprises or consists of the amino acid sequence of SEQ ID NO: 10, and the second Tau peptide immunogen construct comprises or consists of the amino acid sequence of SEQ ID NO: 15.

31. The composition or kit according to any one of claims 1-28, wherein the first Tau peptide immunogen construct and/or the second Tau peptide immunogen construct is selected from SEQ ID NOs: 77-81, optionally wherein the first Tau peptide immunogen construct has the amino acid sequence of SEQ ID NO: 10 and the second Tau peptide immunogen construct is selected from SEQ ID NOs: 77-81.

32. The composition or kit according to any one of claims 1-31, wherein the amyloid beta peptide immunogen construct is selected from SEQ ID Nos: 70, 71 and 89, or a construct in which the positions of the B cell epitope and the Th epitope are reversed.

33. The composition or kit according to claim 32, wherein the amyloid beta peptide immunogen construct comprises or consists of SEQ ID NO: 70.

34. The composition or kit according to claim 32, wherein the amyloid beta peptide immunogen construct comprises or consists of SEQ ID NO: 89.

35. A Tau peptide immunogen construct comprising a B cell epitope comprising an amino acid sequence selected from any one of SEQ ID NOs: 72-76.

36. The Tau peptide immunogen construct of claim 35, which is represented by the formulae: 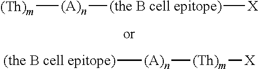
wherein
Th is a heterologous T helper epitope;
A is a heterologous spacer;
(the B cell epitope) comprises, essentially consists of, or consists of an amino acid sequence selected from any one of SEQ ID NOs: 72-76;
X is an α-COOH or α-CONH₂ of an amino acid;
m is from 1 to about 4; and
n is from 0 to about 10.

37. The Tau peptide immunogen construct of claim 35 comprising an amino acid sequence selected from any one of SEQ ID NOs: 77-81.

38. An amyloid beta peptide immunogen construct comprising a B cell epitope comprising, essentially consisting of, or consisting of an amino acid sequence of SEQ ID NO: 82.

39. The amyloid beta peptide immunogen construct of claim 38, which is represented by the formulae: 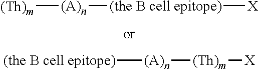
wherein
Th is a heterologous T helper epitope;
A is a heterologous spacer;
(the B cell epitope) comprises, essentially consists of, or consists of the amino acid sequence of SEQ ID NO: 82;
X is an α-COOH or α-CONH₂ of an amino acid;
m is from 1 to about 4; and
n is from 0 to about 10.

40. The amyloid beta peptide immunogen construct of claim 38 comprising, essentially consisting of, or consisting of the amino acid sequence of SEQ ID NO: 89.

41. A composition or kit comprising the Tau peptide immunogen construct according to any one of claims 35 to 37.

42. A composition or kit comprising the amyloid beta peptide immunogen construct according to any one of claims 38 to 40.

43. The composition or kit according to any one of claims 1-34, 41 and 42, which is a single composition comprising the first Tau peptide immunogen construct, and at least one of: the second Tau peptide immunogen construct and the amyloid beta peptide immunogen construct.

44. The composition or kit according to any one of claims 1-34, 41 and 42, which is a kit comprising the first Tau peptide immunogen construct, and at least one of: the second Tau peptide immunogen construct and the amyloid beta peptide immunogen construct.

45. The composition or kit according to claim 44, wherein, prior to administration to a subject, the components of the kit are mixed to create a single composition comprising the first Tau peptide immunogen construct, and at least one of: the second Tau peptide immunogen construct and the amyloid beta peptide immunogen construct.

46. The composition or kit according to claim 44, wherein the components of the kit are used to create a first composition comprising the first Tau peptide immunogen construct, and a second composition comprising at least one of: the second Tau peptide immunogen construct and the amyloid beta peptide immunogen construct.

47. A pharmaceutical composition comprising the composition according to any one of claims 43, 45 or 46, and a pharmaceutically acceptable delivery vehicle and/or adjuvant.

48. The pharmaceutical composition of claim 47, wherein the first Tau peptide immunogen construct, the second Tau peptide immunogen construct and/or the amyloid beta peptide immunogen construct are mixed with an CpG oligodeoxynucleotide (ODN) to form a stabilized immunostimulatory complex.

49. The pharmaceutical composition of claim 47 or 48, which comprises 3 or 4 of Tau peptide immunogen constructs.

50. The pharmaceutical composition of claim 47, 48 or 49, which comprises more than one amyloid beta peptide immunogen constructs.

51. An isolated antibody or epitope-binding fragment thereof that specifically binds to the B cell epitope of the Tau peptide immunogen construct or the amyloid beta peptide immunogen construct obtained by administering to a mammal the composition or the compositions according to any one of claims 43, 45, and 46-50, or the peptide immunogen construct according to any one of claims 35 to 40.

52. The isolated antibody or epitope-binding fragment thereof according to claim 51 bound to the Tau peptide immunogen construct or the amyloid beta peptide immunogen construct.

53. A composition comprising the isolated antibody or epitope-binding fragment thereof according to claim 51 or 52.

54. A method of preventing, inhibiting, reducing the severity of, delaying, or treating a tauopathy in a subject, the method comprising administering to the subject the Tau peptide immunogen construct, the amyloid beta peptide immunogen construct, the composition or the compositions according to any one of the preceding claims.

55. The method of claim 54, which comprises administering the composition wherein the first Tau peptide immunogen construct and at least one of the second Tau peptide immunogen construct and the amyloid beta peptide immunogen construct are comprised within a single composition.

56. A method of preventing, inhibiting, reducing the severity of, delaying, or treating a tauopathy in a subject, the method comprising administering to the subject: a first Tau peptide immunogen construct comprising a first Tau B cell epitope comprising or consisting of 6 to about 40 amino acid residues from the full-length Tau protein of SEQ ID NO: 60; and
(i) a second Tau peptide immunogen construct comprising a second Tau B cell epitope comprising or consisting of 6 to about 40 amino acid residues from the full-length Tau protein of SEQ ID NO: 60, wherein the second B cell epitope is not the same as the first B cell epitope, and/or
(ii) an amyloid beta (Aβ) peptide immunogen construct comprising an amyloid beta B cell epitope comprising or consisting of 6 to about 42 amino acid residues from the full-length amyloid beta protein of SEQ ID NO: 68; and
wherein the first Tau peptide immunogen construct, and the second Tau peptide immunogen construct and/or the amyloid beta peptide immunogen construct, are comprised within separate compositions, which are administered separately.

57. The method according to any one of claims 54-56, wherein the tauopathy is selected from Alzheimer's disease, Lewy body disease, frontotemporal dementia, parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, dementia pugilistica, Down syndrome, Gerstmann-Straussler-Scheinker disease, Hallerworden-Spatz disease, inclusion body myositis, Creutzfeld-Jakob disease, multiple system atropy, Niemann-Pick disease type C, prion protein cerebral amyloid angiopathy, subacute sclerosing panencephalitis, myotonic dystrophy, non-guanamian motor neuron disease with neurofibrillary tangles, postencephalitic parkinsonism, and chronic traumatic encephalopathy.

58. The method according to any one of claims 54-57, wherein the tauopathy is Alzheimer's disease.

59. The method of any one of claims 54-58, wherein the subject is a human.


SEQUENCE LISTING (201 sequences)

SEQ ID NO: 1 - protein, 22 aa, Homo sapiens
MAEPRQEFEVMEDHAGTYGLGD

SEQ ID NO: 2 - protein, 10 aa, Homo sapiens
DHAGTYGLGD

SEQ ID NO: 3 - protein, 12 aa, Homo sapiens
NITHVPGGGNKK

SEQ ID NO: 4 - protein, 17 aa, Homo sapiens
DNIKHVPGGGSVQIVYK

SEQ ID NO: 5 - protein, 12 aa, Homo sapiens
TKIATPRGAAPP

SEQ ID NO: 6 - protein, 16 aa, Homo sapiens
VVRTPPKSPSSAKSRL

SEQ ID NO: 7 - protein, 15 aa, Homo sapiens
IKHVPGGGSVQIVYK

SEQ ID NO: 8 - protein, 16 aa, Homo sapiens
VSGDTSPRHLSNVSST

SEQ ID NO: 9 - protein, 45 aa, synthetic construct
  SITE 26: note = Epsilon Lysine
  SITE 45: note = Amidated Phenylalanine
MAEPRQEFEVMEDHAGTYGLGDKKKKISITEIKGVIVHRIETILF

SEQ ID NO: 10 - protein, 33 aa, synthetic construct
  SITE 14: note = Epsilon Lysine
  SITE 33: note = Amidated Phenylalanine
DHAGTYGLGDKKKKISITEIKGVIVHRIETILF

SEQ ID NO: 11 - protein, 35 aa, synthetic construct
  SITE 23: note = Epsilon Lysine
  SITE 35: note = Amidated Lysine
ISITEIKGVIVHRIETILFKKKKNITHVPGGGNKK

SEQ ID NO: 12 - protein, 41 aa, synthetic construct
  SITE 20: note = Epsilon Lysine
  SITE 41: note = Amidated Lysine
ISITEIKGVIVHRIETILFKKKKKDNIKHVPGGGSVQIVYK

SEQ ID NO: 13 - protein, 35 aa, synthetic construct
  SITE 20: note = Epsilon Lysine
  SITE 35: note = Amidated Proline
ISITEIKGVIVHRIETILFKKKKTKIATPRGAAPP

SEQ ID NO: 14 - protein, 39 aa, synthetic construct
  SITE 20: note = Epsilon Lysine
  SITE 39: note = Amidated Leucine
ISITEIKGVIVHRIETILFKKKKVVRTPPKSPSSAKSRL

SEQ ID NO: 15 - protein, 38 aa, synthetic construct
  SITE 20: note = Epsilon Lysine
  SITE 38: note = Amidated Lysine
ISITEIKGVIVHRIETILFKKKKIKHVPGGGSVQIVYK

SEQ ID NO: 16 - protein, 39 aa, synthetic construct
  SITE 20: note = Epsilon Lysine
  SITE 39: note = Amidated Threonine
ISITEIKGVIVHRIETILFKKKKVSGDTSPRHLSNVSST

SEQ ID NO: 17 - protein, 10 aa, Homo sapiens
MAEPRQEFEV

SEQ ID NO: 18 - protein, 10 aa, Homo sapiens
PRQEFEVMED

SEQ ID NO: 19 - protein, 10 aa, Homo sapiens
RQEFEVMEDH

SEQ ID NO: 20 - protein, 10 aa, Homo sapiens
DHAGTYGLGD

SEQ ID NO: 21 - protein, 10 aa, Homo sapiens
AGTYGLGDRK

SEQ ID NO: 22 - protein, 10 aa, Homo sapiens
GTYGLGDRKD

SEQ ID NO: 23 - protein, 10 aa, Homo sapiens
TYGLGDRKDQ

SEQ ID NO: 24 - protein, 10 aa, Homo sapiens
DRKDQGGYTM

SEQ ID NO: 25 - protein, 10 aa, Homo sapiens
RKDQGGYTMH

SEQ ID NO: 26 - protein, 10 aa, Homo sapiens
KDQGGYTMHQ

SEQ ID NO: 27 - protein, 10 aa, Homo sapiens
GGYTMHQDQE

SEQ ID NO: 28 - protein, 10 aa, Homo sapiens
KIATPRGAAP

SEQ ID NO: 29 - protein, 10 aa, Homo sapiens
IATPRGAAPP

SEQ ID NO: 30 - protein, 10 aa, Homo sapiens
VSGDTSPRHL

SEQ ID NO: 31 - protein, 17 aa, synthetic construct
KKQYIKANSKFIGITEL

SEQ ID NO: 32 - protein, 15 aa, synthetic construct
LSEIKGVIVHRLEGV

SEQ ID NO: 33 - protein, 24 aa, synthetic construct
GAYARCPNGTRALTVAELRGNAEL

SEQ ID NO: 34 - protein, 17 aa, synthetic construct
WVRDIIDDFTNESSQKT

SEQ ID NO: 35 - protein, 23 aa, synthetic construct
DSETADNLEKTVAALSILPGHGC

SEQ ID NO: 36 - protein, 21 aa, synthetic construct
DHEKKHAKMEKASSVFNVVNS

SEQ ID NO: 37 - protein, 17 aa, synthetic construct
KWFKTNAPNGVDEKHRH

SEQ ID NO: 38 - protein, 25 aa, synthetic construct
ALNIWDRFDVFCTLGATTGYLKGNS

SEQ ID NO: 39 - protein, 15 aa, synthetic construct
ISEIKGVIVHKIEGI

SEQ ID NO: 40 - protein, 22 aa, synthetic construct
  VARIANT 7: note = S or T
  VARIANT 10: note = K or R
  VARIANT 11: note = G or T
  VARIANT 15: note = H or T
  VARIANT 16: note = K or R
  VARIANT 19: note = G or T
KKKISIXEIXXVIVXXIEXILF

SEQ ID NO: 41 - protein, 18 aa, synthetic construct
  VARIANT 1..3: note = K or R
  VARIANT 4: note = L, I, V or F
  VARIANT 5: note = F, K or R
  VARIANT 6..7: note = L, I, V or F
  VARIANT 9: note = K or R
  VARIANT 10..11: note = L, I, V or F
  VARIANT 13: note = L, I, V or F
  VARIANT 15: note = Q, L, I or V
  VARIANT 17: note = L, I, V or F
  VARIANT 18: note = D or R
XXXXXXXTXXXTXPXSXX

SEQ ID NO: 42 - protein, 19 aa, synthetic construct
  VARIANT 4: note = S or T
  VARIANT 7: note = K or R
  VARIANT 8: note = G or T
  VARIANT 12: note = H or T
  VARIANT 13: note = K or R
ISIXEIXXVIVXXIETILF

SEQ ID NO: 43 - protein, 18 aa, synthetic construct
  VARIANT 4..5: note = I or F
  VARIANT 6: note = T or L
  VARIANT 7: note = I or L
  VARIANT 11: note = I or L
  VARIANT 14: note = P or I
  VARIANT 15: note = Q or T
  VARIANT 16: note = S or T
  VARIANT 17: note = L or I
KKKXXXXTRIXTIXXXXD

SEQ ID NO: 44 - protein, 19 aa, synthetic construct
ISITEIKGVIVHRIETILF

SEQ ID NO: 45 - protein, 18 aa, synthetic construct
KKKIITITRIITIITTID

SEQ ID NO: 46 - protein, 11 aa, synthetic construct
FVFTLTVPSER

SEQ ID NO: 47 - protein, 15 aa, synthetic construct
SGPLKAEIAQRLEDV

SEQ ID NO: 48 - protein, 9 aa, synthetic construct
DRLRRDQKS

SEQ ID NO: 49 - protein, 19 aa, synthetic construct
AGLTLSLLVICSYLFISRG

SEQ ID NO: 50 - protein, 15 aa, synthetic construct
QYIKANSKFIGITEL

SEQ ID NO: 51 - protein, 20 aa, synthetic construct
PGPLRESIVCYFMVFLQTHI

SEQ ID NO: 52 - protein, 21 aa, synthetic construct
FNNFTVSFWLRVPKVSASHLE

SEQ ID NO: 53 - protein, 16 aa, synthetic construct
KFIIKRYTPNNEIDSF

SEQ ID NO: 54 - protein, 16 aa, synthetic construct
VSIDKFRIFCKALNPK

SEQ ID NO: 55 - protein, 18 aa, synthetic construct
VPGLYSPCRAFFNKEELL

SEQ ID NO: 56 - protein, 14 aa, synthetic construct
DKREMWMACIKELH

SEQ ID NO: 57 - protein, 15 aa, synthetic construct
TGHGARTSTEPTTDY

SEQ ID NO: 58 - protein, 13 aa, synthetic construct
KELKRQYEKKLRQ

SEQ ID NO: 59 - protein, 11 aa, synthetic construct
TVFYNIPPMPL

SEQ ID NO: 60 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 61 - protein, 6 aa, synthetic construct
  VARIANT 3: note = Any amino acid
  VARIANT 5: note = Any amino acid
PPXPXP

SEQ ID NO: 62 - protein, 4 aa, synthetic construct
  SITE 1: note = Epsilon Lysine
KKKK

SEQ ID NO: 63 - protein, 10 aa, Homo sapiens
HAGTYGLGDR

SEQ ID NO: 64 - protein, 45 aa, Homo sapiens
MAEPRQEFEVMEDHAGTYGLGDRKDQGGYTMHQDQEGDTDAGLKE

SEQ ID NO: 65 - protein, 16 aa, Homo sapiens
ADGKTKIATPRGAAPP

SEQ ID NO: 66 - protein, 10 aa, Homo sapiens
PRHLSNVSST

SEQ ID NO: 67 - protein, 30 aa, Homo sapiens
RENAKAKTDHGAEIVYKSPVVSGDTSPRHL

SEQ ID NO: 68 - protein, 42 aa, Homo sapiens [sequence omitted]

SEQ ID NO: 69 - protein, 14 aa, Homo sapiens
DAEFRHDSGYEVHH

SEQ ID NO: 70 - protein, 37 aa, synthetic construct
  SITE 18: note = epsilon Lysine
DAEFRHDSGYEVHHKKKKISITEIKGVIVHRIETILF

SEQ ID NO: 71 - protein, 33 aa, synthetic construct
  SITE 18: note = epsilon Lysine
DAEFRHDSGYEVHHKKKKIITITRIITIITTID

SEQ ID NO: 72 - protein, 15 aa, Homo sapiens
  SITE 1: note = Acetylated Isoleucine
IKHVPGGGSVQIVYK

SEQ ID NO: 73 - protein, 15 aa, Homo sapiens
  SITE 2: note = N-methylated Lysine
IKHVPGGGSVQIVYK

SEQ ID NO: 74 - protein, 15 aa, Homo sapiens
  SITE 1: note = N-methylated Isoleucine
IKHVPGGGSVQIVYK

SEQ ID NO: 75 - protein, 15 aa, Homo sapiens
  SITE 1: note = N-methylated Isoleucine
  SITE 2: note = N-methylated Lysine
IKHVPGGGSVQIVYK

SEQ ID NO: 76 - protein, 15 aa, Homo sapiens
  DISULFID 4..7: note = crosslink between residues
IKHCPGCGSVQIVYK

SEQ ID NO: 77 - protein, 38 aa, synthetic construct
  SITE 1: note = Acetylated Isoleucine
  SITE 19: note = epsilon Lysine
IKHVPGGGSVQIVYKKKKKISITEIKGVIVHRIETILF

SEQ ID NO: 78 - protein, 38 aa, synthetic construct; identical to SEQ ID NO: 15
  SITE 1: note = Acetylated Isoleucine
  SITE 20: note = Epsilon Lysine
  SITE 25: note = N-methylated Lysine

SEQ ID NO: 79 - protein, 38 aa, synthetic construct; identical to SEQ ID NO: 15
  SITE 1: note = Acetylated Isoleucine
  SITE 20: note = Epsilon Lysine
  SITE 25: note = N-methylated Isoleucine

SEQ ID NO: 80 - protein, 38 aa, synthetic construct; identical to SEQ ID NO: 15
  SITE 1: note = Acetylated Isoleucine
  SITE 20: note = Epsilon Lysine
  SITE 24: note = N-methylated Isoleucine
  SITE 25: note = N-methylated Lysine

SEQ ID NO: 81 - protein, 38 aa, synthetic construct
  SITE 1: note = Acetylated Isoleucine
  SITE 4..7: note = Disulfide crosslink between residues
IKHCPGCGSVQIVYKKKKKISITEIKGVIVHRIETILF

SEQ ID NO: 82 - protein, 12 aa, Homo sapiens
  SITE 1: note = pyroglutamic acid
EFRHDSGYEVHH

SEQ ID NO: 83 - protein, 10 aa, Homo sapiens
DAEFRHDSGY

SEQ ID NO: 84 - protein, 12 aa, Homo sapiens
DAEFRHDSGYEV

SEQ ID NO: 85 - protein, 28 aa, Homo sapiens
DAEFRHDSGYEVHHQKLVFFAEDVGSNK

SEQ ID NO: 86 - protein, 10 aa, Homo sapiens
RHDSGYEVHH

SEQ ID NO: 87 - protein, 10 aa, Homo sapiens
HDSGYEVHHQ

SEQ ID NO: 88 - protein, 10 aa, Homo sapiens
DSGYEVHHQK

SEQ ID NO: 89 - protein, 35 aa, synthetic construct
  SITE 1: note = pyroglutamic acid
  SITE 16: note = epsilon Lysine
EFRHDSGYEVHHKKKKISITEIKGVIVHRIETILF

SEQ ID NO: 90 - protein, 352 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 91 - protein, 381 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 92 - protein, 410 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 93 - protein, 383 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 94 - protein, 412 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 95 - protein, 4 aa, synthetic construct
  SITE 4: note = Epsilon Lysine
KKKK

SEQ ID NO: 96 - protein, 15 aa, Homo sapiens
LKHQPGGGKVQIVYK

SEQ ID NO: 97 - protein, 19 aa, synthetic construct
ISISEIKGVIVHKIETILF

SEQ ID NO: 98 - protein, 37 aa, Homo sapiens
VQIINKKLDLSNVQSKCGSKDNIKHVPGGGSVQIVYK

SEQ ID NO: 99 - protein, 31 aa, Homo sapiens
KLDLSNVQSKCGSKDNIKHVPGGGSVQIVYK

SEQ ID NO: 100 - protein, 29 aa, Homo sapiens
DLSNVQSKCGSKDNIKHVPGGGSVQIVYK

SEQ ID NO: 101 - protein, 27 aa, Homo sapiens
SNVQSKCGSKDNIKHVPGGGSVQIVYK

SEQ ID NO: 102 - protein, 25 aa, Homo sapiens
VQSKCGSKDNIKHVPGGGSVQIVYK

SEQ ID NO: 103 - protein, 23 aa, Homo sapiens
SKCGSKDNIKHVPGGGSVQIVYK

SEQ ID NO: 104 - protein, 21 aa, Homo sapiens
CGSKDNIKHVPGGGSVQIVYK

SEQ ID NO: 105 - protein, 19 aa, Homo sapiens
SKDNIKHVPGGGSVQIVYK

SEQ ID NO: 106 - protein, 13 aa, Homo sapiens
HVPGGGSVQIVYK

SEQ ID NO: 107 - protein, 11 aa, Homo sapiens
PGGGSVQIVYK

SEQ ID NO: 108 - protein, 31 aa, Homo sapiens
VQIINKKLDLSNVQSKCGSKDNIKHVPGGGS

SEQ ID NO: 109 - protein, 27 aa, Homo sapiens
VQIINKKLDLSNVQSKCGSKDNIKHVP

SEQ ID NO: 110 - protein, 23 aa, Homo sapiens
VQIINKKLDLSNVQSKCGSKDNI

SEQ ID NO: 111 - protein, 19 aa, Homo sapiens
VQIINKKLDLSNVQSKCGS

SEQ ID NO: 112 - protein, 15 aa, Homo sapiens
VQIINKKLDLSNVQS

SEQ ID NO: 113 - protein, 11 aa, Homo sapiens
VQIINKKLDLS

SEQ ID NO: 114 - protein, 10 aa, Homo sapiens
AEPRQEFEVM

SEQ ID NO: 115 - protein, 10 aa, Homo sapiens
EPRQEFEVME

SEQ ID NO: 116 - protein, 10 aa, Homo sapiens
QEFEVMEDHA

SEQ ID NO: 117 - protein, 10 aa, Homo sapiens
EFEVMEDHAG

SEQ ID NO: 118 - protein, 10 aa, Homo sapiens
FEVMEDHAGT

SEQ ID NO: 119 - protein, 10 aa, Homo sapiens
EVMEDHAGTY

SEQ ID NO: 120 - protein, 10 aa, Homo sapiens
VMEDHAGTYG

SEQ ID NO: 121 - protein, 10 aa, Homo sapiens
MEDHAGTYGL

SEQ ID NO: 122 - protein, 10 aa, Homo sapiens
EDHAGTYGLG

SEQ ID NO: 123 - protein, 10 aa, Homo sapiens
YGLGDRKDQG

SEQ ID NO: 124 - protein, 10 aa, Homo sapiens
GLGDRKDQGG

SEQ ID NO: 125 - protein, 10 aa, Homo sapiens
LGDRKDQGGY

SEQ ID NO: 126 - protein, 10 aa, Homo sapiens
GDRKDQGGYT

SEQ ID NO: 127 - protein, 10 aa, Homo sapiens
DQGGYTMHQD

SEQ ID NO: 128 - protein, 10 aa, Homo sapiens
QGGYTMHQDQ

SEQ ID NO: 129 - protein, 10 aa, Homo sapiens
GYTMHQDQEG

SEQ ID NO: 130 - protein, 10 aa, Homo sapiens
YTMHQDQEGD

SEQ ID NO: 131 - protein, 10 aa, Homo sapiens
TMHQDQEGDT

SEQ ID NO: 132 - protein, 10 aa, Homo sapiens
MHQDQEGDTD

SEQ ID NO: 133 - protein, 10 aa, Homo sapiens
HQDQEGDTDA

SEQ ID NO: 134 - protein, 10 aa, Homo sapiens
QDQEGDTDAG

SEQ ID NO: 135 - protein, 10 aa, Homo sapiens
DQEGDTDAGL

SEQ ID NO: 136 - protein, 10 aa, Homo sapiens
QEGDTDAGLK

SEQ ID NO: 137 - protein, 10 aa, Homo sapiens
EGDTDAGLKE

SEQ ID NO: 138 - protein, 35 aa, Homo sapiens
GSDDKKAKGADGKTKIATPRGAAPPGQKGQANATR

SEQ ID NO: 139 - protein, 10 aa, Homo sapiens
GSDDKKAKGA

SEQ ID NO: 140 - protein, 10 aa, Homo sapiens
SDDKKAKGAD

SEQ ID NO: 141 - protein, 10 aa, Homo sapiens
DDKKAKGADG

SEQ ID NO: 142 - protein, 10 aa, Homo sapiens
DKKAKGADGK

SEQ ID NO: 143 - protein, 10 aa, Homo sapiens
KKAKGADGKT

SEQ ID NO: 144 - protein, 10 aa, Homo sapiens
KAKGADGKTK

SEQ ID NO: 145 - protein, 10 aa, Homo sapiens
AKGADGKTKI

SEQ ID NO: 146 - protein, 10 aa, Homo sapiens
KGADGKTKIA

SEQ ID NO: 147 - protein, 10 aa, Homo sapiens
GADGKTKIAT

SEQ ID NO: 148 - protein, 10 aa, Homo sapiens
ADGKTKIATP

SEQ ID NO: 149 - protein, 10 aa, Homo sapiens
DGKTKIATPR

SEQ ID NO: 150 - protein, 10 aa, Homo sapiens
GKTKIATPRG

SEQ ID NO: 151 - protein, 10 aa, Homo sapiens
KTKIATPRGA

SEQ ID NO: 152 - protein, 10 aa, Homo sapiens
TKIATPRGAA

SEQ ID NO: 153 - protein, 10 aa, Homo sapiens
ATPRGAAPPG

SEQ ID NO: 154 - protein, 10 aa, Homo sapiens
TPRGAAPPGQ

SEQ ID NO: 155 - protein, 10 aa, Homo sapiens
PRGAAPPGQK

SEQ ID NO: 156 - protein, 10 aa, Homo sapiens
RGAAPPGQKG

SEQ ID NO: 157 - protein, 10 aa, Homo sapiens
GAAPPGQKGQ

SEQ ID NO: 158 - protein, 10 aa, Homo sapiens
AAPPGQKGQA

SEQ ID NO: 159 - protein, 10 aa, Homo sapiens
APPGQKGQAN

SEQ ID NO: 160 - protein, 10 aa, Homo sapiens
PPGQKGQANA

SEQ ID NO: 161 - protein, 10 aa, Homo sapiens
PGQKGQANAT

SEQ ID NO: 162 - protein, 10 aa, Homo sapiens
GQKGQANATR

SEQ ID NO: 163 - protein, 45 aa, Homo sapiens
IETHKLTFRENAKAKTDHGAEIVYKSPVVSGDTSPRHLSNVSSTG

SEQ ID NO: 164 - protein, 10 aa, Homo sapiens
IETHKLTFRE

SEQ ID NO: 165 - protein, 10 aa, Homo sapiens
ETHKLTFREN

SEQ ID NO: 166 - protein, 10 aa, Homo sapiens
THKLTFRENA

SEQ ID NO: 167 - protein, 10 aa, Homo sapiens
HKLTFRENAK

SEQ ID NO: 168 - protein, 10 aa, Homo sapiens
KLTFRENAKA

SEQ ID NO: 169 - protein, 10 aa, Homo sapiens
LTFRENAKAK

SEQ ID NO: 170 - protein, 10 aa, Homo sapiens
TFRENAKAKT

SEQ ID NO: 171 - protein, 10 aa, Homo sapiens
FRENAKAKTD

SEQ ID NO: 172 - protein, 10 aa, Homo sapiens
RENAKAKTDH

SEQ ID NO: 173 - protein, 10 aa, Homo sapiens
ENAKAKTDHG

SEQ ID NO: 174 - protein, 10 aa, Homo sapiens
NAKAKTDHGA

SEQ ID NO: 175 - protein, 10 aa, Homo sapiens
AKAKTDHGAE

SEQ ID NO: 176 - protein, 10 aa, Homo sapiens
KAKTDHGAEI

SEQ ID NO: 177 - protein, 10 aa, Homo sapiens
AKTDHGAEIV

SEQ ID NO: 178 - protein, 10 aa, Homo sapiens
KTDHGAEIVY

SEQ ID NO: 179 - protein, 10 aa, Homo sapiens
TDHGAEIVYK

SEQ ID NO: 180 - protein, 10 aa, Homo sapiens
DHGAEIVYKS

SEQ ID NO: 181 - protein, 10 aa, Homo sapiens
HGAEIVYKSP

SEQ ID NO: 182 - protein, 10 aa, Homo sapiens
GAEIVYKSPV

SEQ ID NO: 183 - protein, 10 aa, Homo sapiens
AEIVYKSPVV

SEQ ID NO: 184 - protein, 10 aa, Homo sapiens
EIVYKSPVVS

SEQ ID NO: 185 - protein, 10 aa, Homo sapiens
IVYKSPVVSG

SEQ ID NO: 186 - protein, 10 aa, Homo sapiens
VYKSPVVSGD

SEQ ID NO: 187 - protein, 10 aa, Homo sapiens
YKSPVVSGDT

SEQ ID NO: 188 - protein, 10 aa, Homo sapiens
KSPVVSGDTS

SEQ ID NO: 189 - protein, 10 aa, Homo sapiens
SPVVSGDTSP

SEQ ID NO: 190 - protein, 10 aa, Homo sapiens
PVVSGDTSPR

SEQ ID NO: 191 - protein, 10 aa, Homo sapiens
VVSGDTSPRH

SEQ ID NO: 192 - protein, 10 aa, Homo sapiens
SGDTSPRHLS

SEQ ID NO: 193 - protein, 10 aa, Homo sapiens
GDTSPRHLSN

SEQ ID NO: 194 - protein, 10 aa, Homo sapiens
DTSPRHLSNV

SEQ ID NO: 195 - protein, 10 aa, Homo sapiens
TSPRHLSNVS

SEQ ID NO: 196 - protein, 10 aa, Homo sapiens
SPRHLSNVSS

SEQ ID NO: 197 - protein, 10 aa, Homo sapiens
RHLSNVSSTG

SEQ ID NO: 198 - protein, 30 aa, Homo sapiens
RENAKAKTDHGAEIVYKSPVVSGDTSPHRL

SEQ ID NO: 199 - DNA, 32 bp, synthetic construct
  source 1..32: mol_type = other DNA
  modified_base 1..2: mod_base = OTHER; note = Phosphorothioate linkage
  modified_base 4..5: mod_base = OTHER; note = Phosphorothioate linkage
  modified_base 7..10: mod_base = OTHER; note = Phosphorothioate linkage
  modified_base 12..13: mod_base = OTHER; note = Phosphorothioate linkage
  modified_base 15..18: mod_base = OTHER; note = Phosphorothioate linkage
  modified_base 20..21: mod_base = OTHER; note = Phosphorothioate linkage
  modified_base 23..26: mod_base = OTHER; note = Phosphorothioate linkage
  modified_base 28..29: mod_base = OTHER; note = Phosphorothioate linkage
  modified_base 31..32: mod_base = OTHER; note = Phosphorothioate linkage
tcgtcgttttgtcgttttgtcgttttgtcgtt

SEQ ID NO: 200 - DNA, 24 bp, synthetic construct
  source 1..24: mol_type = other DNA
  modified_base 1..2: mod_base = OTHER; note = Phosphorothioate linkage
  modified_base 4..5: mod_base = OTHER; note = Phosphorothioate linkage
  modified_base 7..10: mod_base = OTHER; note = Phosphorothioate linkage
  modified_base 12..13: mod_base = OTHER; note = Phosphorothioate linkage
  modified_base 15..18: mod_base = OTHER; note = Phosphorothioate linkage
  modified_base 20..21: mod_base = OTHER; note = Phosphorothioate linkage
  modified_base 23..24: mod_base = OTHER; note = Phosphorothioate linkage
tcgtcgttttgtcgttttgtcgtt

SEQ ID NO: 201 - DNA, 24 bp, synthetic construct
  source 1..24: mol_type = other DNA
  modified_base 1..2: mod_base = OTHER; note = Phosphorothioate linkage
  modified_base 4..5: mod_base = OTHER; note = Phosphorothioate linkage
  modified_base 7..10: mod_base = OTHER; note = Phosphorothioate linkage
  modified_base 12..13: mod_base = OTHER; note = Phosphorothioate linkage
  modified_base 15..18: mod_base = OTHER; note = Phosphorothioate linkage
  modified_base 20..21: mod_base = OTHER; note = Phosphorothioate linkage
  modified_base 23..24: mod_base = OTHER; note = Phosphorothioate linkage
tcgtcgttttgtcgttttgtcgtt